(19) United States
(12) Patent Application Publication
(10) Pub. No.: US 2023/0165524 A1
(43) Pub. Date: Jun. 1, 2023

(54) Systems and Methods for Managing A Person's Position to Encourage Proning

(71) Applicant: SMITH & NEPHEW, INC., Memphis, TN (US)

(72) Inventor: Barrett J. Larson, Palo Alto, CA (US)

(73) Assignee: Leaf Healthcare, Inc., Pleasanton, CA (US)

(21) Appl. No.: 17/920,993

(22) PCT Filed: Apr. 8, 2021
(86) PCT No.: PCT/US2021/026341

§ 371 Date: Oct. 24, 2022

Related U.S. Application Data
(63) Continuation-in-part of application No. 15/186,542, filed on Jun. 20, 2016, now Pat. No. 11,051,751.
Application No. 15/186,542 is a continuation-in-part of application No. 13/070,189, filed on Mar. 23, 2011, now Pat. No. 10,729,357.
Application No. 13/070,189 is a continuation-in-part of application No. 15/186,542, filed on Jun. 20, 2016, now Pat. No. 11,051,751.
Application No. 15/186,542 is a continuation-in-part of application No. 14/244,720, filed on Apr. 3, 2014, now abandoned.
Application No. 14/244,720 is a continuation-in-part of application No. 15/186,542, filed on Jun. 20, 2016, now Pat. No. 11,051,751.
Application No. 15/186,542 is a continuation-in-part of application No. 14/543,887, filed on Nov. 17, 2014, now Pat. No. 9,728,061.
Application No. 14/543,887 is a continuation-in-part of application No. 15/186,542, filed on Jun. 20, 2016, now Pat. No. 11,051,751.
Application No. 15/186,542 is a continuation-in-part of application No. 15/028,018, filed on Apr. 7, 2016, now Pat. No. 9,655,546.
(60) Provisional application No. 63/044,475, filed on Jun. 26, 2020.
(60) Provisional application No. 63/015,443, filed on Apr. 24, 2020.
(60) Provisional application No. 61/438,732, filed on Feb. 2, 2011.
(60) Provisional application No. 61/411,647, filed on Nov. 9, 2010.
(60) Provisional application No. 61/393,364, filed on Oct. 15, 2010.
(60) Provisional application No. 61/326,664, filed on Apr. 22, 2010.
(60) Provisional application No. 61/542,785, filed on Oct. 3, 2011.
(60) Provisional application No. 62/047,642, filed on Sep. 8, 2014.
(60) Provisional application No. 61/905,106, filed on Nov. 15, 2013.
(60) Provisional application No. 61/888,078, filed on Oct. 8, 2013.

(51) Int. Cl. A61B 5/00; A61B 5/11; A61B 5/0205; G16H 40/67

(52) U.S. Cl. CPC A61B 5/486; A61B 5/1114; A61B 5/0205; A61B 5/0022; G16H 40/67; A61B 2562/0219; A61B 5/6832

(57) ABSTRACT

Systems, devices, and methods are provided to facilitate the implementation of a “proning protocol” to improve a clinical outcome for a person having SARS-CoV-2 (COVID-19) or other condition that may benefit from spending time in the prone position. For example, a system may include a mobile device (e.g., smartphone, tablet, etc.) providing a proning application configured to manage a configuration and implementation of a proning protocol for a person and configured to receive sensor data from (a) a wearable sensor device secured to the person and including sensor(s) (e.g., accelerometer(s)) that monitor the person body position, and/or (b) other sensor(s) that monitor other physiological parameters relative to the proning protocol. The proning application may determine and output feedback to manage the person's position based at least on the received sensor data and defined parameters of the proning protocol.

### RELATED APPLICATIONS

The present application claim priority to each of the following applications as follows:
(a) The present application is a continuation-in-part of co-pending U.S. Non-Provisional patent application Ser. No. 15/186,542, filed Jun. 20, 2016, which is a continuation-in-part of:
   (i) U.S. Non-Provisional patent application Ser. No. 13/070,189, filed Mar. 23, 2011, which claims priority to the following US Provisional patent applications:
      (a) Ser. No. 61/326,664, filed Apr. 22, 2010;
      (b) Ser. No. 61/373,260, filed Aug. 12, 2010;
      (c) Ser. No. 61/393,364, filed Oct. 15, 2010;
      (d) Ser. No. 61/411,647, filed Nov. 9, 2010; and
      (e) Ser. No. 61/438,732, filed Feb. 2, 2011;
      (ii) U.S. Non-Provisional patent application Ser. No. 14/244,720, filed Apr. 3, 2014, which is a U.S. national stage entry under 35 USC § 371 from international application PCT/US2012/00488 filed Oct. 3, 2012, which claims priority to U.S. Provisional Application Ser. No. 61/542,785 filed Oct. 3, 2011; and
      (iii) U.S. Non-Provisional patent application Ser. No. 14/543,887, filed Nov. 17, 2014, which claims priority to the following US Provisional patent applications:
         (a) Ser. No. 61/905,106, filed Nov. 15, 2013; and
         (b) Ser. No. 62/047,642, filed Sep. 8, 2014; and
      (iv) U.S. Non-Provisional patent application Ser. No. 15/028,018, filed Apr. 7, 2016, which is a U.S. national stage entry under 35 USC § 371 from international application PCT/US2014/59756 filed Oct. 8, 2014, which claims priority to U.S. Provisional Application Ser. No. 61/888,078, filed Oct. 8, 2013; and
   (b) The present application also claims priority to each of the following US Provisional patent application applications:
      (i) Ser. No. 63/015,443, filed Apr. 24, 2020; and
      (ii) Ser. No. 63/044,475, filed Jun. 26, 2020.

The present application claims the benefit of each of the various applications listed above, and the entire contents of each application listed above are hereby incorporated by reference in the present disclosure for all purposes.

### FIELD OF THE INVENTION

Aspects of the present invention relates to systems, devices and methods for implementing and managing a proning regimen for a person, for example a person diagnosed with or experiencing symptoms of COVID-19 or other ARDS (Acute Respiratory Distress Syndrome) condition, to improve oxygenation in the person's respiratory system.

### BACKGROUND OF THE INVENTION

The management of pressure ulcers poses a substantial burden to the healthcare system. Each year, the United States spends billions of dollars treating pressure ulcers and associated complications. Pressure ulcers are very common and they represent a significant source of morbidity and mortality for MPs. The prevalence of pressure ulcers in the US alone is estimated to be between 1.5 and 3.0 million people, with two thirds of cases involving MPs 70 or older.

Pressure ulcers, which are also known as pressure sores, bed sores, or decubitus ulcers, represent localized areas of tissue damage. Pressure ulcers often occur when the soft tissue between a bony prominence and an external surface is compressed for an extended period of time. Pressure ulcers can also occur from friction, such as by rubbing against a bed, cast, brace, or the like. Pressure ulcers commonly occur in immobilized MPs who are confined to a bed, chair or wheelchair. Localized tissue ulceration results when pressure on the skin exceeds capillary filling pressure (approximately 32 mm Hg), which thereby impedes the microcirculation in the skin and the underlying subcutaneous tissue. With compromised blood flow, the delivery of oxygen and nutrients to target tissues is impaired. If blood flow is not restored promptly, the skin and subcutaneous tissue will die and a pressure ulcer will develop.

Pressure ulcers will initially appear as areas of red or pink skin discoloration, but these areas can quickly develop into open wounds if left untreated. Open wounds can lead to severe health complications by exposing MPs to life-threatening infections. The primary goal in the treatment and prevention of pressure ulcers is to relieve pressure on and around affected tissues. Pressure relief can be accomplished by frequently changing the position of immobilized MPs and by using support surfaces that minimize surface pressure. Although pressure management is the most critical aspect of any successful treatment program, it is also important to ensure that MPs receive adequate nutrition, engage in daily exercise, and follow a good skin care and personal hygiene protocol.

A Braden score is commonly used by caregivers to assess an MP's risk for developing a pressure ulcer. The Braden scale is composed of six criteria, which when taken together, can be used to estimate an MP's likelihood of ulceration and can also be used to determine the level of pressure ulcer prevention procedures required for a specific MP. The six components of the Braden scale are: sensory perception, moisture, activity, mobility, nutrition, and friction/shear forces. Each component is rated on a scale of 1 to 4, with the exception of friction/shear which is rated on a scale of 1 to 3. The maximum score is 23, and higher scores reflect a lower risk of developing pressure ulcers. In general, MPs with a Braden score of less than 18 are considered to be at high-risk for developing a pressure ulcer.

Various devices and methods for treating and preventing pressure ulcers have been developed. The cornerstone of pressure ulcer prevention is to turn MPs on a regular basis, such as every one or two hours. MPs confined to a wheelchair, chair, or other surface should be moved in such a manner. Intermittent relief of surface pressure has proven to be highly effective in preventing the development of pressure ulcers. However, various factors limit compliance with turning/repositioning protocols. Some explanations for this low compliance include difficulty monitoring MP position, ineffective turn reminders/alerts, and sub-optimal caregiver staffing ratios—all of which hinder efforts to prevent pressure ulcers. To further exacerbate the problem, as the population ages, the percent of MPs requiring turning is increasing, and yet there is a growing shortage of nursing staff, making it increasingly difficult to maintain compliance with prescribed turning schedules.

Alarm systems have been developed to help improve compliance with turning/repositioning protocols. Generally, these alarms are triggered when the system detects an inadequate amount of MP movement over a predefined time interval. Movement can be detected using various modalities, which include vibration sensors, pressure sensors, and video cameras. Although these systems can detect MP movement, they cannot reliably determine if the perceived movement resulted in adequate depressurization from specific regions of the body.

Also, current alarm systems cannot compute the cumulative pressure-time index (or pressure dose) at specific regions of the body. Although some alarm systems have been designed to measure the surface pressure distribution over a support surface, they are unable to directly correlate the measured pressure with discrete regions of an MP's body. For example, although a pressure sensitive mat placed under an MP can measure the overall surface pressure, it cannot automatically and directly measure the surface pressure at discrete regions of the body, nor can it directly track the cumulative pressure dose at specific regions of the body over time. Furthermore, pressure sensitive mats cannot easily and robustly distinguish between pressure resulting from MP contact with the support surface vs. pressure resulting from non-MP contact with support surface (i.e. books, food trays, etc.).

In addition to turning regimens, pressure ulcer prevention and management also commonly involves the use of pressure reducing support surfaces, which are well known in the art. Such support surfaces attempt to minimize the overall surface pressure and some support surfaces, such as alternating-pressure mattresses, are designed to modulate the surface pressure as a function of time. Although it is desirable to minimize the overall surface pressure, it is important to recognize that different regions of the body have different surface pressure thresholds.

For example, areas underlying bony prominences, such as the hips and sacrum, have relatively low surface pressure thresholds, which is why pressure ulcers commonly occur at these locations. Support surfaces are currently not able to detect or differentiate among specific regions of an MP's body. Without this detection ability, support surfaces are not able to selectively modulate surface pressure at specific regions of an MP's body. Also, current support surfaces cannot automatically identify areas of compromised tissue perfusion, so they are unable to automatically redistribute pressure away from ischemic areas.

There is a need for systems, methods, and devices that help prevent, detect, and/or treat pressure-induced ischemia and pressure ulcers by optimizing surface pressure at areas of compromised tissue perfusion. Various aspects of the present disclosure accomplish these objectives and substantially depart from the conventional concepts and designs of the prior art.

In addition to preventing or managing the development of pressure-induced ischemia and pressure ulcers as discussed above, there are other medical or physiological reasons for managing the position of certain MPs. For example, an increasing number of anecdotal and preliminary scientific reports are indicating that COVID-19 MPs do better when they spend more time in the prone position, referred to as “proning.” Many healthcare providers are encouraging MPs to emphasize proning early in their clinical course, including in the outpatient setting.

“Proning” consists of placing MPs in a face down position for an extended period of time. The technique has been used in the management of MPs with severe respiratory illness since the 1970s. Various reports indicate that proning significantly improves oxygen levels in certain MPs with ARDS (Acute Respiratory Distress Syndrome). Over the past 40 years, numerous high-quality studies have now definitively confirmed that proning improves survival in ARDS2. Today, prone positioning is considered a key component in the management of MPs with ARDS who are intubated and mechanically ventilated. In these MPs, proning has been shown to result in rapid and dramatic improvements in oxygen levels through a variety of mechanisms. These mechanisms include 1) improving respiratory mechanics, 2) improving ventilation, and 3) optimizing blood flow to the lungs.

While proning has been well-validated in the management of ARDS, the technique has traditionally been reserved for intubated, sedated, and critically-ill MPs. However, given the recent surge of respiratory illness due to COVID-19, this technique has been extended to awake, non-intubated MPs who are at risk for clinical deterioration due to coronavirus infection. For example, adopting the prone position for conscious COVID-19 MPs requiring basic respiratory support may benefit such MPs in terms of improving oxygenation, reducing the need for invasive ventilation and potentially even reducing mortality.

The typical supine position of an MPs lying in a hospital bed is known to be detrimental to the MP's underlying pulmonary function. For example, supine positioning may cause (a) over-inflation of the ventral alveoli and atelectasis of the dorsal alveoli (due to an increased trans-pulmonary pressure gradient), (b) compression of alveoli secondary to direct pressure from the heart and the diaphragm being pushed cranially by the intra-abdominal contents, and (c) V/Q Mismatch—As dorsal alveoli are preferentially perfused due to the gravitational gradient in vascular pressures they are poorly ventilation and highly perfused which manifests as hypoxemia. (Peter Bamford et al., ICS Guidance for Prone Positioning of the Conscious COVID MP 2020). Thus, in view of the physiological benefits, proning should be implemented for both intubated and non-intubated MPs. Potential benefits include (a) improved VQ matching and reduced hypoxemia (secondary to more homogeneous aeration of lung and ameliorating the ventral-dorsal trans-pulmonary pressure gradient), (b) reduced shunt (perfusion pattern remaining relatively constant while lung aeration becomes more homogenous), (c) recruitment of the posterior lung segments due to reversal of atelectasis, and (d) improved secretion clearance.

While there is significant evidence supporting the benefits of awake proning for COVID-19 MPs, the potential benefits afforded by this intervention may be limited by poor execution. Unlike with traditional turning protocols, which are often managed by healthcare providers, many MPs are being asked to take responsibility for monitoring and managing their own proning protocols, including in an outpatient setting. Given that MPs are typically unfamiliar with proning protocols, coupled with the fact that many MPs are sick and often physically isolated to minimize cross-contamination, there is a need for automated systems and methods for implementation and management of MP proning protocols.

### SUMMARY OF THE INVENTION

The present disclosure provides systems, methods and devices for MP management, including the detection, treatment and prevention of wounds such as pressure ulcers, among other things, and conditions likely to cause such wounds. Furthermore, the present disclosure provides communication from one or more sensors monitoring an MP to a host system to alert caregivers to key conditions and to enable an improved, more reliable method for MP care. In some embodiments, the sensor can be self-contained without the need for communication to a host, in other embodiments the sensor can indicate MP position and turn information directly on the sensor, whether self-contained or in communication with a host. Alternatively, the host system can initiate an automated care event. Some aspects of the present invention relate to sensing systems that locate sites of compromised tissue perfusion or tissue injury and substantially optimize surface pressure at those locations.

As used herein, the term “monitored person” or “MP” includes any person to which any of the systems, devices, and methods disclosed herein may be applied, and not limited based on the medical or treatment status of the person, e.g., not limited based on (a) whether the person is at home, at a medical treatment or care facility (e.g., hospital, medical clinic, or assisted living home) in an inpatient or outpatient capacity, or at any other location, (b) whether or not the person is under the care of a doctor or other medical professional, (c) whether the person has received a medical evaluation or diagnosis, etc.

Other aspects of the present disclosure relate to sensing systems that provide information regarding the position, orientation, and/or movements of an MP, and allow for surface pressure optimization based on this information. Here the position refers to the shape that the body takes independent of orientation, for example, knees bent, back straight, arms above head. The orientation refers to direction that the body is facing and the angle, for example, supine, prone, rotated left, rotated right, tilted Trendelenburg, tilted reverse Trendelenburg, etc. Movement refers to changes in either position, location, or orientation, achieved by bending, translating, or turning, respectively. Such sensors can be placed directly on the body, or on or in the support surface, or on or in clothing worn by the MP, or can be sensors capable of monitoring MPs from more remote locations. In a presently preferred arrangement, a sensor comprising a multi-axial accelerometer provides data representative of MP position, orientation, and movement, which is then processed by a host system, which can be remote from the sensor, as described hereinafter.

Other aspects of the disclosure provide techniques for selectively modulating surface pressure at and around sites of compromised tissue perfusion, or sites of tissue injury, or sites considered to be at risk for developing tissue injury or sites where pressure is not desirable, thus substantially eliminating at least some of the conditions likely to lead to the formation of pressure ulcers, as well as aiding in the treatment of pressure ulcers and other wounds.

Still other aspects of the present disclosure comprise the use of body surface markers together with systems and techniques for optimizing surface pressure at locations corresponding to such body surface markers. For example, body surface markers can be placed over areas of damaged tissue or areas thought to be at high-risk for developing pressure sores (i.e. hips, heels, sacrum, etc.). The support system can then attempt to focus pressure-relieving maneuvers at and around these locations. Body surface markers can include, but are not limited to, the following: stickers, wound dressings, socks, undergarments, and sensible ink or other media, films, or adhesives. Depending upon the implementation, body surface markers can be comprised of anything that has at least one sensible property that is in some way distinguishable from the MP by a host system. As used herein, “sensible” means “capable of being sensed.” In at least some embodiments of the present disclosure, pressure distribution over time and location is then selectively optimized with respect to the body surface markers in an effort to optimize tissue perfusion.

Still further aspects of the present disclosure are configured to minimize or eliminate physical contact with injured tissue, areas of compromised tissue perfusion, areas identified to be at-risk for compromised tissue perfusion, or areas corresponding to body surface markers. An objective of an embodiment of the present disclosure is to control the surface pressure at sites of tissue injury, sites identified as having compromised tissue perfusion, or sites corresponding to body surface markers. These aspects of the disclosure allow for increased blood circulation and increased airflow to critical areas, thus promoting the healing of existing pressure ulcers and preventing the formation of other pressure ulcers.

Some embodiments provide a lightweight multi-function sensor that can be easily affixed to an MP. In an embodiment, the sensor includes a multi-axis accelerometer, together with other logic for monitoring MP turns and related physical characteristics of the MP. In another embodiment, the sensor further includes a magnetometer and an altimeter. Further, the sensor communicates with a network of receivers, which may in an embodiment be relay antennas forming a mesh network. The sensor can, in at least some embodiment, be a lightweight, single use, disposable sensor affixed to the MP by means of a medical adhesive.

As discussed in greater detail hereinafter, MP orientation can be determined by the proper placement of the sensor on the torso of an MP. In one embodiment, the sensor comprises in part a three-axis accelerometer which, together with the algorithms disclosed herein, determines the orientation of the MP. Indicia on the sensor is used in some embodiments to simplify determining the orientation of the sensor on the MP.

In an embodiment, a system of the present disclosure comprises a host system for receiving data from a wireless sensor, together with storage for maintaining historical information about the MP. The historical MP data is used in connection with the algorithms processed in a processing unit to make recommendations to a caregiver, or, in the case of automated care systems, to enable the effectuation of the automated care. MP-specific data can be provided to the system by any or all of being developed algorithmically, heuristically, or through manual entry.

Some disclosed embodiments employ a mesh network for monitoring of various MP activities including MP orientation, location, potential or actual bed exits, and falls. In at least some embodiments, the addition of a sensor to the network, and the self-enabling structure of the network elements, can be achieved automatically, although manual configuration is also possible in some embodiments.

One embodiment of the sensor also includes a magnetometer and an altimeter. Small, low-power versions of such devices are available from a plurality of sources. The magnetometer permits determination of the direction an MP is facing, particularly when sitting up. A sudden change in magnetometer reading can indicate an MP attempting a bed exit. For many MPs with limited mobility, the process of exiting the bed proceeds slowly, such that an alarm triggered by virtue of the change in magnetometer reading (which can in some embodiments be combined with accelerometer and altimeter data) allows a caregiver time to reach the MP before a bed exit actually occurs.

In some embodiments, a wearable sensor device includes indicators such as LEDs that can indicate which side the MP is on, when an MP requires a turn, which area of the body has been exposed to the most pressure, which direction an MP should be turned onto, or when an MP has been turned sufficiently to satisfy a turning protocol or to depressurize a given area.

In another aspect of the disclosure, MP self-roll or repositioning can be encouraged by various means, such as audio, visual or physical/tactile guidance. In a related aspect, acceleration and orientation monitoring of the MP may be used to monitor for motion caused by an alternating pressure mattress. Some embodiments provide a monitoring system that, by detecting MP accelerations, can determine if an MP is being repositioned sufficiently. In some embodiments, the system can include a pressure measurement system which can produce a pressure map of reasonable precision that then feeds back to a support surface.

In another aspect, the system can automatically calculate at least one suggested decompression threshold/interval. The decompression threshold/interval refers to the minimum amount of time that an area of the body needs to experience reduced pressure or no pressure in order to adequately re-profuse that area of the body, thereby preventing ischemia and tissue damage. Some embodiments can also detect system can also detect very low to no movement or situations in an MP, such as when the MP's breathing, heartbeat, and other physical motions have stopped.

Some embodiments of the present invention are configured to facilitate a “proning” regimen for a person, e.g., to improve a clinical outcome for a person having a condition or experiencing symptoms that may benefit from spending time in a prone position. For example, some embodiments provide automated or partially automated systems, device, and methods for selecting, configuring, and/or implementing (executing) a proning protocol for a monitored person (“MP”), e.g., for improving clinical outcomes of MPs with ARDS, other respiratory conditions, or other medical conditions that may benefit from spending time in a prone position. For example, systems, devices, and methods are provided for monitoring and managing the position of an MP with COVID-19 or other ARDS condition (or experiencing symptoms associated therewith), for example with respect to a proning protocol designed to encourage or “coach” the MP to spend time in a prone position, for example, to improve a clinical outcome for the MP.

Some embodiments or situations apply to a conscious, non-intubated MP, while others apply to an intubated or otherwise unconscious MP. Some embodiments provide a system including:
(a1) at least wearable sensor secured to an MP and including one or more sensor (e.g., one or more accelerometer, magnetometer, etc.) configured to monitor the MP's orientation over time (e.g., prone, supine, lying on right side, or lying on left side, the MP's torso inclination angle, and/or any other measure of orientation) configured to generate sensor data for monitoring the physical position/orientation of the MP over time, and in some embodiments, sensor(s) configured to measure or monitoring other physiological parameters relevant to implementing and managing a proning protocol for the MP (e.g., MP temperature, oxygenation data, heart rate, respiratory rate, respiratory sounds, etc.); and/or
(a2) at least one other sensor or medical device configured to monitor additional physiological data regarding the MP, e.g., body temperature, respiratory rate, respiratory sounds, heart rate, heart rate variation (HRV), SpO2, SaO2, FiO2, PaCO2, SBP, etc. For example, the system may include a fingertip pulse oximeter, an audio sensor (e.g., for monitoring respiratory sounds), an EKG sensor, and EEG sensor, a ventilator and associated sensors (in the case of an intubated MP), CPAP machine, BiPAP machine, or other oxygen therapy machine or device for providing the MP supplemental oxygen and/or respiratory assistance; and
(b) a mobile device (e.g., smartphone, tablet, smart watch, etc.) configured to wirelessly communicate with the wearable sensor(s) and providing a proning user application configured to manage a selection, configuration, and/or implementation (execution) of a proning protocol for the MP. The proning user application may receive user input and/or sensor data received from the (a) at least wearable sensor secured to the MP (including data indicating the body position/orientation of the MP) and/or (b) the at least one other sensor or medical device (if present) configured to monitor physiological data regarding the MP, and make relevant decisions specified by the proning protocol, such as determining the current position/orientation of the MP, determining when to instruct or recommend a position change (e.g., based on body position timers maintained for each respective body position), determining whether to remove a particular body position (e.g., lying on the left side), whether to discontinue the proning protocol, and whether and how to adjust one or more parameters of the proning protocol. The proning user application provide feedback to the MP in any suitable manner, e.g., via a display screen of the mobile device or via audible or haptic feedback output by the mobile device or by the wearable sensor(s), e.g., to instruct or encourage the MP to change positions (e.g., from or to the prone position), or to give feedback regarding the MP's compliance with or progression through the proning protocol.

### BRIEF DESCRIPTION OF THE DRAWINGS

FIG. 1A illustrates in block diagram form an embodiment of an example system in which one or more sensors provide to a host data representative of an MP's position, orientation, and movement, and the host uses that information, together with other MP information, to identify risks with respect to either avoidance or treatment of pressure ulcers on the MP, among other things.

FIG. 1B illustrates in block diagram form an embodiment of the hardware of a system in accordance with one embodiment of the disclosure.

FIG. 1C illustrates in block diagram form an embodiment of a system wherein the sensor forms a key part of a system for detecting and monitoring bed exits, falls, ambulation, location, orientation, and vital signs.

FIG. 1D illustrates the relative size of a wireless sensor in accordance with an embodiment of the disclosure.

FIG. 2 illustrates in flow diagram form an embodiment of the process flow for comparing new sensor data from an MP with historical MP information for the purpose of preventing or treating pressure ulcers on the MP, and capable of running on the system of FIG. 1B.

FIG. 3A illustrates an accelerometer-based sensor in accordance with one aspect of the disclosure.

FIG. 3B illustrates an alternative design of the sensor of FIG. 3A, which permits indicating pressure and turn information directly on the sensor, and can, in some embodiments, be self-contained.

FIG. 3C illustrates in flow diagram form the operation of the pressure and turn indication functions of the sensor of FIG. 3B.

FIG. 3D illustrates the progression of pressure and turn indication as an MP is turned from laying on his right side to laying on his left side.

FIG. 3E illustrates a variety of different sensor shapes that can provide the pressure and turn indication functions of the sensor of FIG. 3B.

FIG. 3F illustrates an alternative design of the sensor of FIG. 3A or FIG. 3B, which includes a local (e.g., bedside) monitor/display device in communication with the MP-worn sensor and configured to display MP orientation information based on sensor data generated by the MP-worn sensor.

FIG. 4A illustrates the processing of signals from a sensor as shown in FIGS. 3A and 3B to determine at least orientation.

FIG. 4B illustrates in flow diagram form application of a correction factor to align acceleration data with a rotational axis of the body.

FIGS. 5A-5B illustrates the orientation of x-y-z axes relative to an MP using a sensor as shown in FIGS. 3A-3B.

FIG. 6 illustrates a sample response of the x-y-z accelerometers due to a ninety degree turn, or roll, by an MP, such as turning from a supine position to lateral decubitus position.

FIG. 7 illustrates in flow diagram form the filtering steps used to isolate orientation, heart rate, breathing rate and movement data from the raw accelerometer signals, including feedback paths for improving filtering.

FIG. 8 illustrates in flow diagram form an embodiment of a filter in accordance with this aspect of the disclosure.

FIG. 9 illustrates a variety of indices applied to the sensors of FIGS. 3A-3B for ensuring proper location and orientation on the MP.

FIG. 10 illustrates two arrangements of electrodes for the sensors of FIG. 3A-3B, the first comprising seven electrodes including common, and the second comprising three electrodes including common.

FIG. 11 illustrates an electrode orientation by which only two electrodes are required when spaced at a known angle.

FIG. 12 illustrates in block diagram form the functional hardware architecture of one embodiment of a sensor, and its communication to a caregiver through a mesh network and a host.

FIG. 13A shows various network configurations for monitoring MP orientation in accordance with some embodiments of the disclosure.

FIG. 13B illustrates in block diagram form the data acquired and processed in the various functional blocks in accordance with an embodiment of the disclosure.

FIGS. 13C and 13D show an embodiment of the interaction of the MP-worn sensor with a host through a mesh network formed by a plurality of relay antennae, with the resulting data being ultimately provided to a caregiver through a display such as a computer, a tablet, or a smartphone.

FIG. 14 illustrates a process for powering up devices not connected to a power grid, such as MP sensors, and then integrating them into a network as appropriate in some embodiments of the disclosure.

FIG. 15 illustrates a process for powering up network devices such as mesh network transceivers/antennas connected to a power grid and integrating them into a network in a manner appropriate to some embodiments of the disclosure.

FIG. 16 illustrates a device main loop, such as used by a sensor in accordance with some embodiments of the disclosure, and by which status updates including, for example, MP position, are provided to the system.

FIG. 17 illustrates an example process for monitoring body region compression and decompression times.

FIG. 18 illustrates an example system flow for detecting and recording an MP's rotation about the cephalo-caudal axis.

FIG. 19 illustrates an example system flow for detecting and recording an MP rotating while also sitting up partially or entirely.

FIG. 20 illustrates an example system flow for detecting whether a sensor is properly affixed to an MP.

FIG. 21 illustrates an example flow diagram of a process flow for determining if an MP bed-exit is likely to occur, a bed-exit has occurred, and/or a fall has occurred.

FIG. 22 illustrates an embodiment of a process flow for determining if an MP bed-exit is likely to occur.

FIG. 23 illustrates an embodiment of a process flow for determining if an MP bed-exit has occurred using accelerometer and altimeter information.

FIG. 24 illustrates an embodiment of a process flow for determining if an MP bed-exit has occurred using location information.

FIG. 25 illustrates an embodiment of a process flow for determining if an MP fall has occurred using altimeter information.

FIG. 26 illustrates an embodiment of a process flow for determining if an MP fall has occurred using altimeter and accelerometer information.

FIG. 27 shows an example splash screen for the user interface of the system.

FIG. 28 shows an example user interface screen for assigning a sensor to a unit within a hospital.

FIG. 29 shows an example table for recording sensor assignments to MPs.

FIG. 30 illustrates an example user interface screen for a particular MP being monitored by an example system according to one embodiment.

FIG. 31 shows an example user interface screen for an unassigned sensor.

FIG. 32 shows an example user interface screen for stopping MP logging upon discharge.

FIG. 33 shows an example user interface screen for pausing turn alerts.

FIGS. 34A-34B illustrate one embodiment of a system flow for entering and recording pauses of turn alerts.

FIG. 35 is a user interface screen for logging, manual, an MP turn.

FIG. 36 is a user interface screen illustrating an embodiment for verifying sensor attachment.

FIGS. 37A-37B show example user interface screens showing various administrative settings.

FIGS. 38A-38B illustrate an embodiment in which indicators such as LEDs are provided on the sensor so as to be visible through the housing of the sensor, for assisting in indicating MP position.

FIGS. 39A-39B illustrate an example embodiment in which the MP is encouraged to self-roll.

FIG. 40 illustrates an example embodiment in which identification of the caregiver is implemented.

FIG. 41 illustrates an example embodiment in which deep vein thrombosis risk can be monitored.

FIG. 42 illustrates an example implementation 4200 of a system for monitoring and managing a proning regimen for a monitored person (MP), according to one example embodiment.

FIG. 43 illustrates another example implementation 4300 of a system for monitoring and managing a proning regimen for one or more MPs, in which the MPs may be monitored by a caretaker, according to one example embodiment.

FIG. 44 illustrates another example implementation 4400 of a system for monitoring and managing a proning regimen for one or more MPs, in which the MPs may be remotely monitored by a caretaker, according to one example embodiment.

FIG. 45 illustrates an example implementation 4500 that utilizes multiple types of sensors or devices for collecting data regarding an MP for managing a proning protocol for the MP, according to example embodiments.

FIG. 46 illustrates an example arrangement 4600 of a wearable sensor 4320 configured to communicate with a user device (e.g., smart phone) 4210 for monitoring and managing a proning regiment for an MP, according to one example embodiment.

FIG. 47 illustrates selected components of an example user device 4210 (e.g., a smart phone or other computer device) running an iOS or Android operating system, according to example embodiments.

FIGS. 48A-48D illustrate example screen views generated by proning user application 4220 and displayed via GUI 4676 at user device 4210, according to one example embodiment.

FIG. 49 illustrates an example method 4900 for implementing and managing a proning protocol for an MP, according to one example embodiment.

FIG. 50 illustrates an example method 5000 for implementing and managing a proning protocol for an MP, according to one example embodiment.

FIG. 51A shows an example process flow 5100 for determining whether to implement, and implementing, an example proning protocol for a conscious MP.

FIG. 51B shows an example progression of body positions defined by an example proning protocol implemented with reference to the process flow of FIG. 51A, according to one example embodiment.

### DETAILED DESCRIPTION OF THE INVENTION

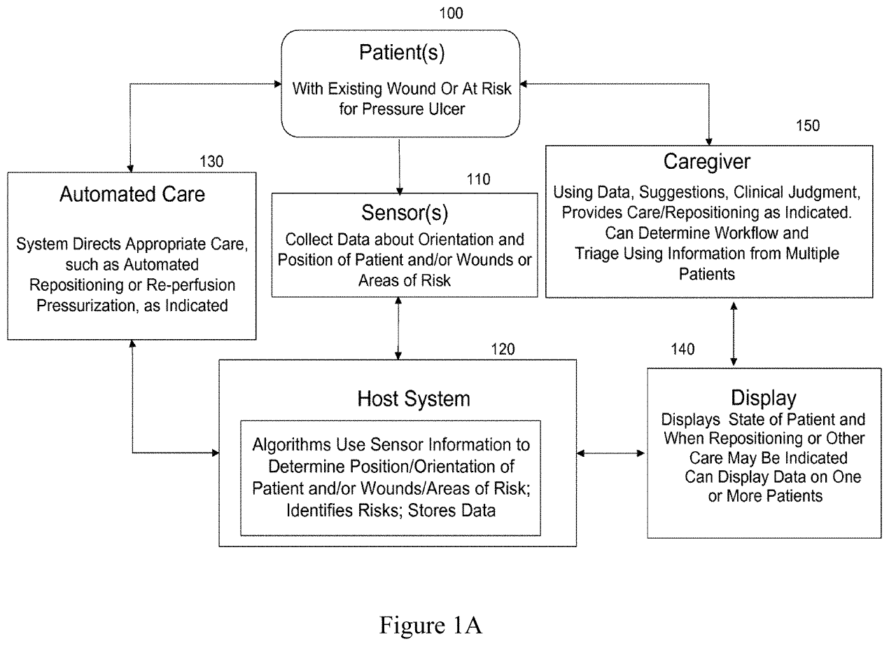

Some embodiments disclosed herein provide an improved means for managing and coordinating turning protocols. FIGS. 1A-1B show an example system in which an MP 100 requiring monitoring, and in at least some instances having an existing wound or being at risk for developing a pressure ulcer, is associated with one or more sensors 110. The sensors collect data about the orientation, position, and movement of the MP and/or wounds and/or areas of compromised tissue perfusion and/or areas of risk. The sensors communicate with a host system 120, typically a computer running at least one program for processing the incoming sensor information to determine the position or orientation or movements of an MP, wounds or areas of compromised tissue perfusion, or areas of risk on the MP. The program also uses historical and other data to analyze the sensor data and identify risks. In at least some embodiments, the data, including both the sensor data and the analytical data, is stored for future use.

Depending upon the embodiment, the output of the host system can provide direction to an automated care system, as shown at 130, or can display messages for the attention of a caregiver as shown at 140. In the latter instance, the caregiver uses the suggestions from the system together with training and judgment and makes a determination regarding management of an MP's care, as shown at 150.

Referring more specifically to FIG. 1B, an embodiment of the hardware components of the system of FIG. 1A can be better appreciated. More specifically, the sensors 110, a variety of which are described in greater detail hereinafter, collect MP orientation and physiologic data. In some instances, this can include heart rate, respiratory rate, and other data in addition to MP orientation, position, and movement. The host system 120 typically comprises a processing unit 125 together with at least one data storage device. The processing unit executes one or more software programs to analyze the sensor information and determine the state of the MP, to determine care recommendations based on the current state of the MP and relevant stored data, and, in some instances directs the operation of an automated care system 130. The data store 135 typically comprises a hard disk, RAM, EEPROM, solid state disk, or other memory device, and stores current and historical sensor data, health status of the MP, wound locations if any, at risk locations if any, as well as recommendations and settings for MP care. In some systems, the data store can be integrated with or linked to one or more of the hospital's databases, such that data in the data store 135 is updated whenever the hospital records are updated. The host system 120 communicates by either wired or wireless links with the display 140 and/or one or more automated care systems 130.

With reference to FIGS. 1C and 1D, in an embodiment, a sensor 110 comprises a multi-axis accelerometer, for example a three-axis accelerometer, a magnetometer and an altimeter. Such a combination, typically in combination with a host network comprising a mesh network of relay antennae and at least one server/host, enables the detection and monitoring of MP orientation 155 while in bed, to permit the use of a personalized turn protocol. The combination can further detect MP orientation 155, vital signs 160, bed exits 165, falls 170, MP location 175 and MP ambulation 180.

As shown in FIG. 1D, an example sensor 110 comprises a small, lightweight, single use, disposable device having enclosed within a housing that is affixed to an MP. The sensor is typically affixed by means of a medical adhesive to the upper torso of the MP, such as at the sternum or below a clavicle. The sensor can, in some embodiments include indicia for orienting the sensor relative to body, e.g., with the top of the indicia pointing toward the head of the MP.

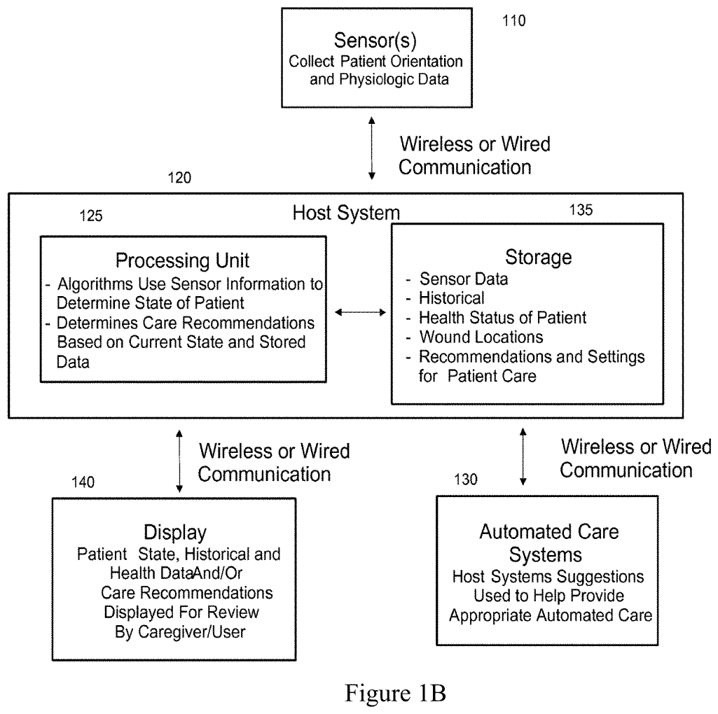

Referring next to FIG. 2, the operation of the software component(s) of the system of FIGS. 1A-1D can be better appreciated. Data 200 from the sensor is initially filtered and analyzed, as shown at step 205, to determine if the sensor is both used and functioning properly. That determination is made at step 210; if the sensor is not functioning properly, a notice about the deficiency is sent at step 215. However, if the sensor is functioning properly, the process continues at step 220, where the raw sensor data is filtered and analyzed to determine the orientation of the MP. Next, at steps 221-223, a check is made to determine whether the MP has exited the bed, or is in a position to imminently exit the bed, or is standing, or is ambulating. Such checks can be made as described in co-pending U.S. patent application Ser. No. 14/543,887, filed 17 Nov. 2014 and incorporated herein by reference. Sensing modalities that can be used to make such determinations include one or more of accelerometers, magnetometers, altimeters, and general location sensing techniques (i.e. triangulation and sensor position localization), each used singly or in multiples.

In the event that the MP is either about to exit the bed, or has exited the bed to stand, or is out of the bed and ambulating, an adjustment to the pre-existing turn protocol is appropriate in some embodiments. In each instance, the MP either is not or soon will not be supported by the support surface. Two adjustments may be appropriate to the turn protocol, depending upon the embodiment. First, the MP does not need to be turned, and, second, the tissues that were recently pressurized when the MP was on the support surface will now start to depressurize. Further, the rate of such depressurization will typically exceed the rate of depressurization if the MP had remained on the support surface and been turned as described herein. This faster rate of depressurization occurs because: 1) the body tissues are not under any pressure from a support surface, 2) the cardiac output is likely increased with ambulation and thus tissue perfusion is improved relative to a sedentary MP, and 3) MPs that are ambulatory are generally at lower risk for pressure ulcers and thus require a less stringent turning protocol.

In some implementations, detection of ambulation (or standing) is cause to immediately reset the turn clock and amend MP history to reflect immediate depressurization of all body tissues. In other implementations, detection of ambulation (or standing) will cause the body tissues to depressurize at an accelerated rate; that is, the depressurization will take much less time. For example, if tissues depressurize at a rate of 1× when on a support surface, the same tissue may depressurize at a rate of 2× upon ambulation. The adjustment to depressurization rate or time can be varied based on MP-specific data, or can be a fixed value, or any other suitable arrangement, and the turn protocol is adjusted to reflect the need [or lack thereof] for a current turn as well as the change in depressurization rate. MP-specific data related to such adjustments can be, for example, how often the MP exits the bed, how long they stand, how far and how quickly they ambulate, falls or a lack thereof when out of bed. an MP who exits the bed once a week for a few steps may be assigned a different depressurization rate or time, and turn protocol, than an MP who ambulates daily for 100 feet.

In some embodiments, detection of ambulation (or standing) is cause to temporarily disable, suspend, or otherwise discontinue the MP's turning schedule. When an MP is not supported on a support surface, there is no need for the MP to be turned or repositioned and therefore the turning protocol (and all associated alerts/notifications) can temporarily be suspended. As soon as it is determined that the MP has returned to a support surface, the turning protocol (and all associated alerts/notifications) can be resumed. In some embodiments the amount of depressurization that has occurred while the turn protocol has been suspended can be taken into account when the turn protocol is resumed.

If the MP is not about to exit the bed, nor standing, nor ambulating, the process advances directly to step 225 with no adjustments to the depressurization rate or the existing turn protocol. If an adjustment has been made at step 242, the process advances to step 225 with that adjustment implemented for the further steps.

Then, at step 225, an orientation-based pressure map is generated, followed at step 230 by a pressure-time determination to assess how long areas of tissue have been subjected to a given pressure. A time input can be derived from the host 120, or a separate time base can be used to make the pressure-time measurement. Then, at step 235, the pressure-time measurement is compared to a preset limit, and, together with historical data, how long the area has been depressurized, when the most recent depressurization of the area occurred, health conditions of MP, location of wounds, areas of risk, and other factors, together with historical positioning data as shown at step 240, a determination is made regarding suggested repositioning.

Then, at step 245, a determination is made as to whether the data suggests that the MP should be repositioned soon. If no, the process ends at step 250, with, in some embodiments, the display of orientation, position, and movement data and a suggested repositioning schedule. If yes, and an automated care function exists as checked at 251 and is required as checked at 253, the decision at step 245 results in a directive to provide automated care at step 255. Alternatively, or in the event that automated care is not successful or is not required, a message is sent to a caregiver at step 260 advising of the need for repositioning, as well as a suggested new position. In the event automated care is available to the caregiver as an option, the caregiver either accepts the suggestion, indicated at 265, or provides alternate care at step 270 based on judgment and training.

Sensors that may be used in the various systems disclosed herein can vary widely, and may include sensors both in continuity with the MP's body or remote to the MP's body. Possible sensors include accelerometers, RFID sensing, resistive, capacitive, inductive and magnetic sensors, reflective sensors, infrared sensors, video monitoring, pressure and stress sensors, transcutaneous oxygen pressure sensors, transcutaneous CO2 sensors, hydration sensors, pH sensors, ultrasound sensors, remote optical spectroscopy sensors, and laser Doppler flow sensors, among others.

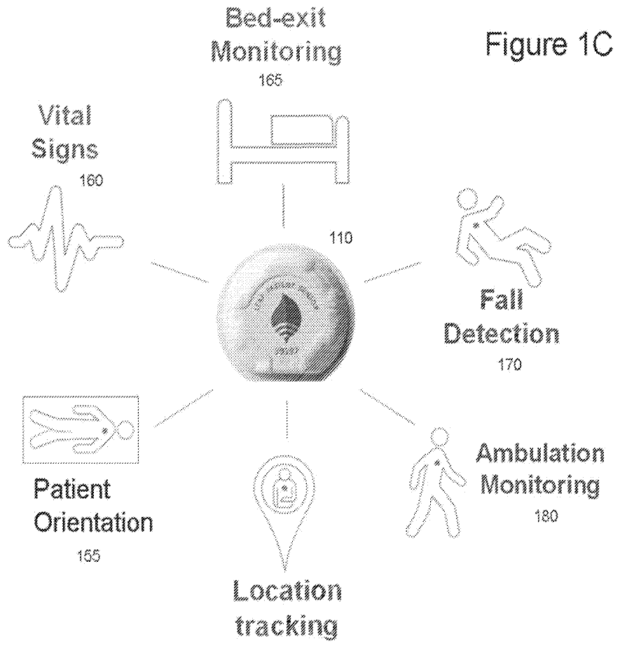

As shown in FIG. 3A, one embodiment of a sensor comprises a multi-axial accelerometer 305 with associated processor 310 and related electronics, and generally indicated by 300. One acceptable accelerometer is the type LIS344ALH three axis accelerometer available from ST Microelectronics, although sensing on three axes is not required in all embodiments. In addition to the accelerometer, the sensor 300 can also comprise a capacitive sensor 315, a temperature sensor 320, a moisture sensor 325, and an electrical signal sensor 330. The microprocessor 310 can comprise a built-in A/D converter and stored sensor identifier, and communicates with a base station/host 335 which can include a transceiver for wireless communications, located near enough to reliably receive wired or wireless signals, through an RF transceiver 340 and antenna 345. Alternatively, the transceiver/base station 335 communicates with a remote host. In either case, the host ultimately links to viewing terminals 350 that can be, for example, integrated into the MP sensor or support system, in the MP room, at the nursing station, or at other locations. It will be appreciated that, while not shown, a battery or other power source is provided in the sensor 300. It will be appreciated by those skilled in the art that the functions of the host can reside in several different locations in a system as disclosed herein. For example, the host functionality can largely reside in the sensor itself, or that functionality can coexist within the base station, or it can be external to both, or the functions can be split across multiple devices.

In an embodiment of the sensor, the device is stored such that battery life is preserved until the unit is put into use. Alternatively, the sensor is designed with a rechargeable battery or other energy storage device such as a capacitor. A rechargeable sensor can be recharged by connecting with a cable to some other energy source such as a power converter or can be recharged wirelessly through the use of an inductive charger. A non-rechargeable system may have lower cost and be more suitable for one-time disposable use in a hospital or other short-term care environments while a rechargeable sensor may have greater initial cost but may be more economical in a long term-care facility, such as a nursing home. The sensor can be activated by, for example, removing the adhesive backing on the unit, or by a conventional switch, or by exposure to ambient light in the MP's room, or activated upon exposure to an MP. Alternatively, the sensor can be activated by passive RFID, which can be built into the unit itself or embedded in the adhesive backing of the unit. The sensor can also be active by RF or inductive loops. Precautions are also typically taken to protect the sensor's accelerometers. Precautions can be taken, for instance, to prevent damaging accelerative forces from acting on the accelerometer. In an embodiment, the casing of the sensor unit can be compressible so as to decrease the accelerative force of a fall or impact. Alternatively, or additionally, the accelerometer can show when an acceleration large enough to cause damage or a need for recalibration is experienced and the senor unit can then signal that it is damaged or in need of calibration. In other embodiments, the sensor can also include an additional accelerometer capable of sensing accelerations greater than the acceptable range for a primary accelerometer, to be used to measure accelerations that can damage or cause a requirement for recalibration in a more sensitive accelerometer. In an accelerometer with more than 2 axes, all 3 axes can be used to determine orientation, providing more than one calculation of orientation that can be compared and used as an indicator that an accelerometer is damage or in need of recalibration

The sensor, together with other system components as shown in, for example, FIG. 1, can provide real-time monitoring of an MP's orientation and surface pressure distribution over time, whereby MPs requiring intervention can easily be identified. One embodiment utilizes small, thin, inexpensive, wireless and disposable sensors that safely monitor the 3-dimensional orientation of an MP over time. In one embodiment, the sensors have an adhesive backing, such that they can be affixed to the MP's body. In an embodiment, one or more sensors can be placed on the body at known anatomic locations, although the anatomical location of the sensor(s) is not required to be known in some alternative embodiments, as explained in greater detail hereinafter. The sensors can be placed on the body in a location that does not increase the risk for tissue damage. In one instantiation of this embodiment, a small sensor is affixed to the sternum or the anterior superior iliac spine (ASIS) of the MP. The sensors can also be embedded in articles worn by the MP, such as shirts or underwear bracelets, belts, or collars, as long as the sensor does not move significantly relative to the MP.

The sensors used in the present embodiment can contain one or more accelerometers, gyroscopes, magnetometers, or other devices, which are capable of measuring one or more conditions of the MP. The accelerometer can reliably and accurately measure MP tilt, MP orientation, MP movement, and vibration, and shock, as would occur with a fall. The accelerometer can be coupled to a wireless transmitting device, such that there are no wires extending from the MPs to whom the sensors are attached. Wireless communication can be achieved via radio frequency transmission. Monitoring the wireless communication from the body sensors enables real-time tracking of the condition of the MP, including MP orientation and orientation-based pressure distribution over time. Alternatively, wireless communication can be implemented using an infrared or other optical link.

The present embodiment can be used to accurately monitor the static angle and acceleration of MPs relative to the support surface. By continuously measuring the MP's orientation relative to the support surface a system can determine to what extent the MP needs to be repositioned and/or the extent to which a next-scheduled turn can be skipped or delayed. Warnings can be given in response to a predefined condition, such as prolonged MP position at a specific angle relative to the support surface. The sensor data can be transferred to a central location that manages a network of monitored MPs to ensure that all MPs are being repositioned adequately. The network can be used to provide warnings to caregivers and to coordinate repositioning schedules amongst caregivers.

The sensors and monitoring system described in this embodiment are able to track the cumulative amount of time that an MP has been in a specific orientation relative to a support surface. The system can also estimate the surface pressure exerted on different regions of the body based on the direction of the gravitational force vector (as determined by the accelerometer), the orientation of the support surface, and the estimated magnitude of that force vector (as defined by physical attributes of the MP, such as height, weight, BMI, mass distribution, etc.). A computer can analyze the MP orientation/surface pressure data over time for each MP, and recommend optimal repositioning maneuvers based on this data. Furthermore, the cumulative surface pressure distribution for each MP can be seamlessly tracked and recorded as the MP moves to and from different support surfaces (i.e. bed, chair, wheelchair, couch, etc.). Information regarding each MP's pressure ulcer history, Braden score, and other conditions of the MP can be entered into the monitoring system. The computer can recommend an optimal repositioning schedule based on MP-specific data.

An alternative embodiment of the sensor of FIG. 3A is shown in FIG. 3B, with like elements having like reference numerals. In this embodiment, a multi-axis magnetometer 355 is included, and supplements the operation of the sensor of FIG. 3A with directional information by providing its output to the processor 310. An altimeter 357 can also be included in the embodiment of FIG. 3B to provide elevation data, which can be used to anticipate or record bed exits, falls, or ambulation, all as discussed in greater detail in copending U.S. patent application Ser. No. 15/036,782, filed 2016 May 13 and incorporated herein by reference. Those skilled in the art will appreciate that the magnetometer and altimeter are not necessary to every embodiment of the sensor of FIG. 3B. Further, the processor 310 outputs to LED drivers and LEDs 360, the operation and function of which are explained in connection with FIGS. 3C and 3D. Unlike the sensor of FIG. 3A, in some embodiments the sensor of FIG. 3B can operate independently of a host/base station—that is, it can operate as a self-contained orientation sensor and turn indicator—and thus the inclusion of an RF transceiver and antenna is optional for the sensor of FIG. 3B.

Referring next to FIG. 3C, the function and operation of the LED Drivers and LEDs of FIG. 3B can be better appreciated. For convenience of illustration, the sensor of FIG. 3C will be assumed to be a self-contained sensor, although the sensor could also be configured to communicate with a host as noted above. In particular, the sensor is placed on an MP and then activated, as shown in step 361 although the order is not critical. Depending upon the embodiment, the sensor can have preloaded a standard turn protocol, an MP-specific turn protocol, a standard Head of Bed (“HOB”) bed elevation protocol or an MP specific HOB protocol. Then at step 365, an optional calibration process is performed such as described hereinafter, for example for determining and correcting for chest angle, roll, pitch or yaw, or proper orientation on the MP. A caregiver such as a nurse then awakens the sensor from low power mode, for example by tapping the sensor twice or by any other suitable means, as shown at 369. After a period of time, as shown at 373, the sensor has monitored the MP's position, including changes in orientation, using the accelerometer and, optionally, the magnetometer and the altimeter to provide inputs to the processor 310. In addition, the processor with its associated storage has recorded the length of time the MP has spent in one or more orientations. During this period, the LEDs are typically not energized and the sensor is operating in a low power mode to conserve the battery. However, the processor uses MP history together with the sensed orientation data and any stored or embedded turn or elevation protocol to develop an alert pattern that can be illuminated by LED's 381, as shown in the various examples indicated at 381A-D. For this illustrative embodiment, a pattern of 13 LEDs forms a circular pattern in a sensor housing. It will be appreciated by those skilled in the art that the number of LEDs is not critical, nor is the circular pattern although a circular pattern has some advantages as discussed in connection with FIG. 3D. Alternative arrangements of LEDs on a sensor housing are shown in FIG. 3E.

The LED Drivers and LEDs can, under the control of the processor When in the initial verification and calibration mode, all LEDs are lit as shown at 381A. In the low power mode, shown at 381B, the LEDs are all off. Then, when the sensor is awakened from low power mode, a pattern of LEDs illuminates to reflect the region of the MP most subject to pressure. Thus, as shown at 381D, a single LED is blinking to indicate the region of greatest pressure, flanked on either side by at least one illuminated LED indicating that the associated region of the MP is also pressurized even if not to the same degree as the primary region. Depending upon the embodiment, only the LED's reflecting regions of the MP's skin that are significantly pressurized may be illuminated, with the rest off, or the other LEDs may be illuminated differently, for example either a different color or a different brightness, or a slow blink, or any other suitable manner to distinguish them from the LEDs indicating significant regions of pressurization of the MP's skin. In some embodiments, a caregiver alert can be generated by the processor independently of any input from a caregiver, for example when a turn or other form of attention to the MP is needed. Such an alert can be a blinking illumination of all LED's, as shown at 381C, or other suitable indication intended to attract the attention of a caregiver.

In at least some embodiments, the data from the sensor, and especially any data causing a caregiver alert, is stored in non-volatile memory in the sensor so that the cause of the alert can be determined even if the battery is allowed to run down.

For purposes of this example, assume that the LEDs illuminate as shown in FIG. 381D and indicate that a turn is required for the MP to comply with the applicable protocol. A check is made at step 385 to determine whether a change in MP position has been satisfied, or other applicable time-out has occurred. If not, the process loops to step 373, where MP position is again determined and the appropriate LEDs are illuminated. However, if MP position requirements are satisfied or a time-out has occurred, the process advances to step 389 where it reverts to low power mode until its next awakening, again either by a caregiver input (e.g., a double tap or other suitable input) or an alert causing the processor to illuminate the LEDs without caregiver input, shown at 369.

Referring next to FIG. 3D, the use of the LEDs in the sensor of FIG. 3B to both reflect MP position and guide the caregiver through a turn to a safe position can be better understood. Again, for convenience of illustration, a circular arrangement of LED's is used, although the alternative sensor/LED arrangements of FIG. 3E, elements 399A-399D, can also provide similar indications. Continuing with FIG. 3D, an MP resting on his right side is due for a turn, with the area of the MP's skin subject to pressurization indicated at his right hip as shown. Whether automatically or by caregiver input, the LEDs 393A illuminate to show a primary region of pressurization, indicated by the blinking LED, flanked on one side by two solidly (non-blinking) illuminated LEDs and flanked by one solidly illuminated LED on the other side.

Either through caregiver input, or on the MP's own initiative, the MP begins a turn from his right side onto his left side. As he turns, the blinking LED indicating primary pressure moves over by one LED, such that now there is only one solidly lit LED on the one side where there were two, shown at 393B. As the turn continues, the blinking LED continues to change position in the circular array, and now all of the solidly lit LEDs are on the other side of the blinking LED representing the primary region of pressurization. As the turn continues, shown at 393D, the blinking LED has moved farther around the circular array than any of the originally lit LED's. To indicate the region that was pressurized until the turn began, the four LED's indicating that region remain solidly lit. As the turn continues, 393E, the blinking LED showing continues to move around the circle, and now is separated from the solidly lit LEDs by one unlit (or differently lit) LED. Finally, as shown at 393F, the MP completes the turn onto the left side, and no LEDs are lit adjacent the LED indicating the primary region of pressurization. In some embodiments, the LED's indicating the prior region of pressurization will stop being illuminated once the MP reaches the new position. In other embodiments, those LEDs will remain lit (at least when the sensor is awakened) for a depressurization period to indicate that that region has not yet been adequately depressurized. In such embodiments, the LEDs may be controlled by the processor and LED drivers to be a different color, or less brightly lit, or to slowly fade in accordance with depressurization status, or any other suitable arrangement. The legend shown in FIG. 3D is provided solely as an example of possible uses of color, blinking, solidly lit, and so on.

Different configurations of LEDs on a sensor can be appreciated from FIG. 3E, although the examples shown are merely indicative of the many options available and are not intended to be limiting. The example shown at 399A retains the circular pattern discussed above, while 399B includes a circular pattern together with a vertical strip (or horizontal, the orientation is a matter of choice) of a plurality of LED's. A strip of circular LED's is shown at 399C, while 399D shows a plurality of strip LEDs arranged vertically along a sensor in the shape of a rectangular strip.

FIG. 3F shows another alternative embodiment (e.g., alternative to the embodiments of FIGS. 3A and 3B) including wireless sensor 300 connected to a local monitor 336 and/or to one or more terminals 350 (discussed above) communicatively connected to sensor 300 by any suitable wired and/or wireless communication links.

Local monitor 336 may comprise a monitor and/or display device located local to the MP-worn sensor 300. As shown, local monitor may include a processor 337 and LED drivers and LEDs 360 such as described above regarding FIG. 3B, such that LED electronics 360 are located in the local monitor 336 separate from the MP-worn sensor 330 instead of being provided by the MP-worn sensor 330 itself. Thus, LED electronics 360 provided by local monitor 336 may be configured to display or indicate various orientation data regarding the MP based on data communicated from the sensor 300, including current orientation data indicating a current orientation of the MP and/or historical orientation data, e.g., indicating an amount of time spent in one or more different orientations (e.g., based on a previous instance of the MP in each respective orientation, or a cumulative amount of time spanning multiple instances of the MP in each respective orientation).

The orientation of the MP may be monitored by MP-worn sensor device 300 or by local monitor 336, or both. Sensor data generated by one or more sensors of sensor device 300 may be processed by processor 310 of MP-worn sensor device 300 and/or by processor 337 of local monitor 336.

For example, in some embodiments, processor 310 of sensor device 300 may process sensor signals to monitor the orientation of the MP over time, e.g., by determining the current orientation data and/or historical orientation data of the MP over time. Sensor device 300 may communicate such current orientation data and/or historical orientation data to the local monitor 336, and processor 337 of the local monitor 336 may control LED electronics 360 based on this received current orientation data and/or historical orientation data, to display or indicate the current orientation of the MP and/or historical information regarding one or more orientations (e.g., cumulative time spent in one or more different positions).

As another example, in some embodiments, sensor device 300 may communicate the raw sensor signals to local monitor 336, and processor 337 may then process the sensor signals to monitor the orientation of the MP over time, e.g., by determining the current orientation data and/or historical orientation data of the MP over time based on the received sensor signals, and control LED electronics 360 based on this determined current orientation data and/or historical orientation data.

In other embodiments, each of MP-worn sensor device 300 or by local monitor 336 may independently process the sensor signals generated by sensor 300 and monitor the MP orientation, or MP-worn sensor device 300 and local monitor 336 may cooperate to process the sensor signals and monitor the MP orientation. For example, in some embodiment, processor 310 of sensor 300 performs some pre-processing of the sensor data generated by the onboard sensor(s) (e.g., accelerometer data), and wirelessly communicates this pre-processed sensor data to local monitor 336, where processor 337 performs additional processing of the data to determine and monitor the MP orientation. This pre-processing by the sensor device 300 may compress or reduce the volume of data, thereby reducing the volume of wireless data transmission from sensor 300 to local monitor 336. The additional power consumption at sensor 300 required for the pre-processing of the sensor data may be outweighed by the reduction in power consumption at sensor 300 resulting from the reduced volume of wireless data transmission.

Thus, it should be understood that the processing of sensor signals and monitoring of the MP orientation of time may be performed by MP-worn sensor device 300 or by local monitor 336, or both.

In some embodiment, local monitor 336 may be located within the same room as the MP or within range for a direct wired connection (e.g., by a cable connected between the MP-worn sensor 300 and local monitor 336) or within the range of a particular type of short-range wireless communication used between the MP-worn sensor 300 and local monitor 336, e.g., WiFi, RF, Bluetooth, RFID, near-field communication (NFC), infrared, ZigBee, etc.

In some embodiments, local monitor 336 may be a bedside monitor secured to or securable to the MP's bed. For example, local monitor 336 may comprise bedside receiving station 30 disclosed in parent U.S. patent application Ser. No. 12/730,663, including the details of bedside receiving station 30 described at pages 5 and 9-14 (including the section titled “3. Bedside Receiving Station”) of the original specification filed on May 24, 2010, which details are incorporated by reference herein.

In one embodiment, the sensing system is properly secured to the MP in order to accurately determine the MP's orientation and surface pressure distribution. In one embodiment, the system comprises means for automatically determining if the sensor system is properly attached to the MP. A system that can detect and notify the caregiver when the sensor is not attached, not attached properly, not oriented on the MP properly, not located on the MP properly, or is otherwise not working properly is desirable. Such a condition, if not detected, can result in the MP being in an orientation sufficiently long to develop a pressure ulcer or experience some other adverse medical condition. Depending upon the embodiment, any of several methods may be employed to verify proper location, orientation, and operation of the sensor. One set of embodiments comprises means and method for detecting biometric parameters that indicate if the orientation sensor is properly secured to the MP. In this approach, the orientation sensor is considered properly attached to the MP only when detected biometric parameters fall within predefined values based on known physiological behavior. If the detected biometric parameters fall outside of predefined limits, then the MP orientation sensor is considered to be improperly secured to the MP, or not attached to the MP, and caregivers can be alerted. The detected biometric parameters can include, but are not limited to, skin capacitance, respiratory rate, heart rate, and temperature. In the event of any error condition, where the measured parameters are out of range, the system notifies the caregiver that the system or more specifically, the sensor or base station is not working properly

Another method to determine if the sensor is functioning properly is to range-check the raw data collected by the sensor. In the case of a sensor that is measuring acceleration in three axes, the magnitude of the acceleration or the components of acceleration that exceed a predefined maximum or minimum reasonable acceleration would indicate that the accelerometer or interface electronics are not working properly. In the case of other types of sensors, raw resistance, raw capacitance, raw inductance, etc. can be range checked against reasonable minimum and/or maximum values. The sensor can also monitor circuit voltage levels and current levels, battery voltage and battery current draw, battery charge state and report anomalous values to the base station. The sensor can have and compare multiple time bases, for example, more than one clock, oscillator, and/or timer. If the time bases give different values for elapsed time then the sensor can report anomalous values to the base station. Alternatively, a sensor with a single time base can compare elapsed time against a time base located in the base station.

An additional method for detecting if a sensor is not working properly is to compare the computed orientation, or location at a point in time or a range of orientations or locations over time against what might reasonably be expected. For example, if the computed orientation is an orientation that is impossible for the MP to assume then the sensor is likely not working properly. A paralyzed MP that is computed to suddenly change from a supine to a prone position may indicate a problem with the sensor. A sensor that rotates more than a prescribed maximum angular deviation, for example, 180 degrees in any plane, may indicate a failed sensor. A range of angular deviations and orientations can be identified such that, if the sensor is found to be outside of range, an error is indicated. Similarly, a sensor that assumes more than a prescribed maximum angular acceleration may indicate a failed sensor. A range of orientations that is unexpected or a computed orientation that is unexpected could also indicate that a sensor has been attached to the wrong body location. For example, a body extremity, such as the foot can assume orientations and undergo a range of orientations that is different than those for the pelvis or thorax.

A properly working RF communication link between the sensor and the base station, and between the base station and the nursing station, can be verified at a regular interval by communicating an expected message between these separate system components at prescribed intervals. Failure to receive the proper message at the proper time indicates the failure of the communication link.

Bio-metric data collected by the sensor can be used to verify its proper attachment, location, and/or function. For example, even if the primary purpose of the sensor is to collect orientation data, the sensor can also measure pulse rate, respiratory rate, skin capacitance, optical properties, or other physical properties of the MP to verify that the sensor has been properly attached, oriented, positioned, and/or is functioning properly.

Proper operation of sensors such as that illustrated in FIG. 3B, which can operate independently of a host and thus are self-contained, can be verified by, for example, a nurse or other caregiver determining that the illuminated LEDs accurately reflect MP position, or by a self-test sequence that can be initiated, for example, by a tactile input from the caregiver, such as a triple tap or other sequence.

The sensing system described herein can be used to measure an MP's respiratory rate. As the chest rises and falls during respiration, a sensor 300 placed on or near the MP's thorax will undergo a cyclic pattern of acceleration/deceleration. The computer system, which may include appropriate software as described herein, can interpret this cyclic pattern of acceleration/deceleration as a respiratory rate when it fits into physiologic parameters associated with human breathing, including but not limited to the rate, amplitude, and waveform of the accelerations/decelerations. In an embodiment, the system can be designed such that it uses the respiratory rate to ensure that the sensor is properly affixed to the MP's body. If the system does not detect a respiratory rate, it can be interpreted that the MP is apneic or the sensor may have fallen off the MP or the sensor may not be properly attached to the MP. If the system detects an abnormal respiratory pattern (which can include abnormal breathing rate and/or abnormal magnitude of chest rise/fall during respiration), it can be interpreted that the MP is in respiratory distress. The system can identify abnormal breathing patterns, such as hyperventilation, periodic respirations, sighing, air trapping, etc. If an abnormal respiratory pattern is detected, caregivers can immediately be alerted via an alarm mechanism.

In a similar fashion, the sensing system described herein can be used to measure an MP's heart rate. As the heart beats in the chest cavity, a sensitive accelerometer placed on or near an MP's thorax will undergo a cyclic pattern of accelerations/decelerations. A cyclic rise and fall of the chest wall that is within physiologic limits (including, for example, amplitude, frequency, and waveform consistent with a physiologic heart rate) can be measured by an accelerometer 305 and can be interpreted by the system of FIG. 1, for example, to be the MP's heart rate. The system can be designed such that it uses the heart rate to ensure that the sensor is properly affixed to the MP's body. If the system does not detect a heart rate, it can be interpreted that the MP is in cardiac arrest or the sensor may have fallen off the MP or the sensor may not be properly attached to the MP. If the system detects an abnormal heart pattern or arrhythmia (which can include abnormal heart rate and/or abnormal magnitude of chest rise/fall during a heartbeat), it can be interpreted that the MP is in cardiac distress. The system can identify abnormal heart patterns or arrhythmias, such as tachycardia, bradycardia, fibrillation, etc. If an abnormal heart pattern or arrhythmia is detected, caregivers can immediately be alerted via an alarm mechanism. The sensor may also contain an embedded electrical activity sensor that is capable of detecting the electrical activity of the heart. The sensor can also be correlated with an EKG in order to increase the sensitivity/specificity of the monitoring system.

The MP orientation and surface pressure monitoring system described herein can be designed to automatically feedback directly into the pressure control system of MP support surfaces. Many support surfaces are capable of regulating surface pressure at discrete locations. By providing the pressure control system with information regarding the MP's position, orientation, location, movements, and surface pressure distribution over time, the surface pressure of the support surface can be optimized. The surface pressure can also be regulated such that the MP is automatically rolled or repositioned to relieve pressure on any high-risk areas.

Depending upon the implementation, the sensing system described herein can be designed for home care, nursing care, or ambulatory care monitoring, without requiring direct caregiver support. The sensor can be worn by an MP (either affixed to their skin or embedded in an article of clothing) and the orientation/surface pressure distribution of the MP can be monitored either constantly or periodically. If the system detects the potential for pressure-induced injury, an audible and/or visual alarm can go off. The alarm can notify the MP of the need to change position/orientation, and upon doing so, the alarm can automatically turn off. The alarm can be programmed to turn off only if the MP repositions themselves sufficiently. In one embodiment, the alarm system described herein can be programmed to have increasing levels of audio or visual stimulation. For example, when the system detects that repositioning is indicated, a low-intensity sound can be produced by the system. If the MP does not reposition themselves, the intensity of the sound can increase until the MP has sufficiently repositioned themselves. If the MP is unable to reposition him or herself, then caregivers can be alerted. The sensing system described herein can be used as a telemedicine MP monitoring solution.

The MP orientation and surface pressure monitoring system described herein can be used to help prevent SIDS (Sudden Infant Death Syndrome). An infant position/orientation sensor is able to detect if an infant is lying facing up or face down on a support surface. Recommendations are in place for infants to sleep face up, so as to prevent accidental asphyxiation. The sensor unit can be used to inform caregivers when an infant, or any other person, is lying prone. The sensor can inform or alert caregivers when the infant is in a predefined orientation relative to a support surface and can also remotely send data to caregivers, such as via phone, pager, or computer system. The MP monitoring system may be capable of also measuring heart rate, respiratory rate and breathing patterns by analyzing movement of the chest wall. Information regarding respiratory rate and/or breathing pattern can be displayed and/or correlated with infant or MP position/orientation to increase the specificity of detecting potentially harmful orientations. The MP orientation sensor can be affixed directly to the MP's skin, or embedded in an article of clothing, such as a diaper or pajamas. An embedded temperature sensor can also be used to determine the skin surface temperature of the user. The sensing system can also monitor the physical location of the user, and indicate if the user has fallen, is walking, is rolling, is crawling, etc.

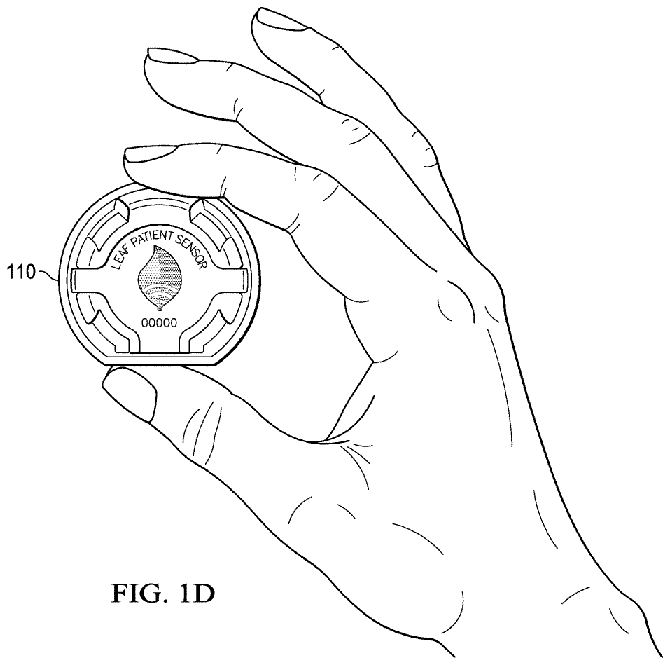

The sensor 300 not only detects accelerations due to changes in an MP's position/orientation, but also accelerations due to heartbeats, breathing, other movements, etc. To improve the detection of MP an MP's position/orientation, it is desirable to separate sensor signals caused by changes in an MP's position/orientation from acceleration signals caused by other forces including breathing, heartbeats, etc. To determine an MP's position/orientation, only the acceleration due to gravity is needed. At the same time, it can be useful in some embodiments to be able to monitor heart rate, breathing, and other vital signs with the sensor 300, as discussed in greater detail hereinafter. To determine MP position/orientation, it is desirable to filter out signals due to other sources, and this can be accomplished by the use of a low pass filter since MP turns are typically slow compared to other movements detected by the sensor 300. An example of a cutoff frequency for the filter can be 0.1 Hz (since the lower end of normal respiratory rates is approximately 0.2 Hz), though other frequencies can be used. Other methods for isolating the gravitational accelerative forces include taking the average, median, mode, or some combination of these of the accelerative signal over several readings. These methods allow for approximately removing the higher frequency and more random, less constant, or more cyclical accelerative forces. Components of the signal that give acceleration above 1 g can also be removed as noise, since the gravitational acceleration does not likely exceed 1 g for a user at rest. An additional method for isolating low frequency accelerations is to include an inertial mass on the accelerometer swing arm to reduce its inherent responsiveness to high frequency movement. Such an arrangement is shown in FIG. 4A, where the raw signal 400 from the accelerometer is passed through one or more filters 405 for isolating the accelerative force due to gravity. Once the proper signals are isolated, the MP's position/orientation can be determined successfully, as at 410.

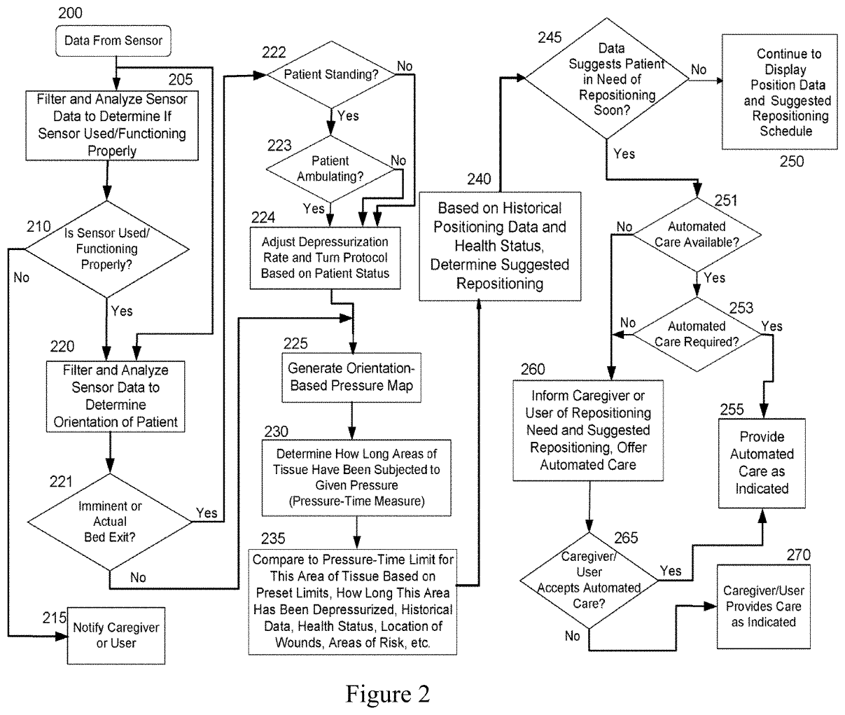

The method for using the sensor 300 to determine orientation can be better appreciated from FIGS. 5A-5B. The sensor is attached to the user such that the orientation of the user is measured by the accelerative forces experienced by the accelerometer. The separate axes of the multi-axis accelerometer are often oriented orthogonally relative to each other, and shown in FIG. 5B. Shown in FIG. 5A is a 3-axis accelerometer with one axis (x in this case) aligned along the cephalic-caudal axis of the user, another (y) aligned along the left-right axis, and another (z) aligned along the anterior-posterior axis. The side-to-side rotation of the user is picked up by the z and y-oriented accelerometers. The Trendelenburg and reverse Trendelenburg tilts of the user (head to toe tilt) are picked up by the x and z oriented accelerometers. As such, it may be redundant to have more than 2 orthogonal axes sensed by accelerometers. However, the redundancy can be used for several purposes including: confirming the orientation calculation, and using different accelerometers for different angles of orientation to allow for the accelerometers to operate in their most accurate angle zones.

Consider an example, where the user is tilted 30 degrees to the right side. The component of gravitational acceleration along the x-axis accelerometer does not change. However, it does change on the y-axis and z-axis. With the MP lying flat, the z-axis accelerometer experiences the maximum acceleration due to gravity in the downward/posterior direction, as it is parallel to the direction of gravity. The y-axis accelerometer experiences minimal gravitational acceleration as it is perpendicular to gravity. As the user tilts to the right, the component of gravity experienced by the z-axis accelerometer decreases and the component of gravity experienced by the y-axis accelerometer increases. When the user reaches 30 degrees of tilt to the right side, the z-axis accelerometer experiences approximately cosine(30)g of gravitational acceleration. At this orientation, the y-axis accelerometer experiences sine(30)g=0.5 g of gravitational acceleration. For other orientations involving tilting about the x-axis/cephalic-caudal axis, the acceleration experienced by the z and y accelerometers will follow a similar relationship where 30 is replaced by the angle of tilt. Similarly, if the user is tilted in the Trendelenburg or reverse-Trendelenburg positions, the z-axis accelerometer experiences approx. cosine(angle)g of gravitational acceleration and the x-axis accelerometer experiences sine(30 angle)g of gravitational acceleration. By knowing the gravitational acceleration experienced by the accelerometers, one can then find the angle of the tilt. In the case of a simple tilt where there is only tilting about one axis, this can be accomplished by taking the arc-sine or arc-cosine of the ratio of the measured acceleration due to gravity to magnitude of gravitational acceleration.

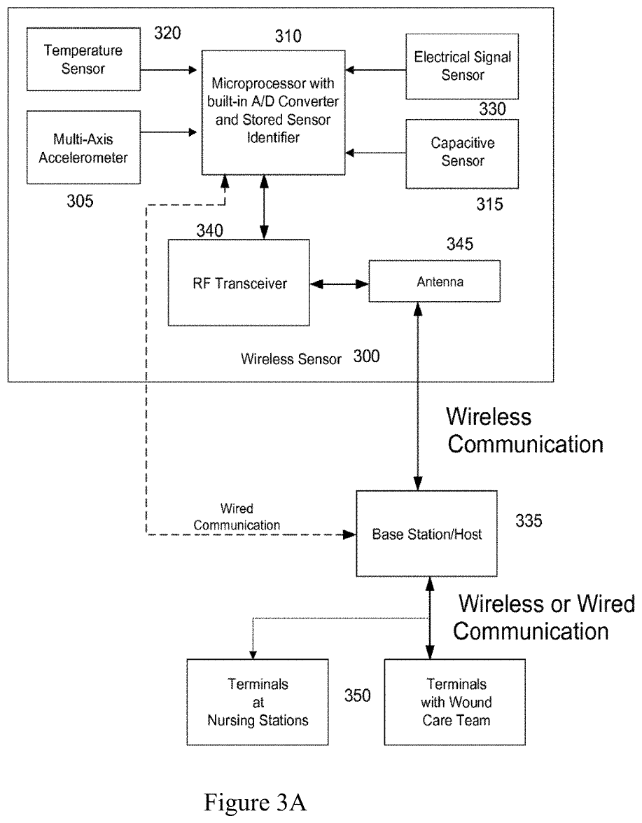

FIG. 6 shows sample data from a 3-axis accelerometer showing a 90 degree turn of a user. The z-axis accelerometer initially shows a 1 g acceleration when the sensor is flat and then shows approximately 0 g when the sensor is at 90 degrees. Note that in FIG. 6 the acceleration is not in units relative to g but as output from the accelerometer. The opposite is true for the y-axis accelerometer. In the case of tilting about more than one axis, the component of the gravitational acceleration experienced by the accelerometers is reduced compared to a non-tilted state. For instance, if the user is in the reverse-Trendelenburg position (head tilted up relative to feet) by 5 degrees, then when the user is now tilted side-to-side (i.e. about the x axis), the z-axis accelerometer experiences cos(5 degrees)*g instead of the full g. As the user is tilted about the x-axis, the z-axis acceleration measurement continues to be decreased at a ratio of cos(5 deg). Similarly, for the y-axis during side-to-side rotation (about the x-axis), the y-axis gravitational acceleration measured is decreased at a ratio of cos(5 deg). A similar calculation is used for any angle of inclination of the z-axis, replacing the 5 degrees by the angle of inclination. Similarly for a rotation about the x-axis, the gravitational acceleration measured by the z and y-axis are decreased at a ratio of cos(angle of rotation).

In general usage, if there is tilting about more than one axis, the user is tilted in the Trendelenburg or reverse-Trendelenburg position and being rotated about the x-axis. In this case, the x-axis acceleration can be used to determine the angle of tilt about the y-axis using techniques as described above. This angle of tilt is then used in the calculation of the rotation about the x-axis, by dividing the ratio of the experienced acceleration by the magnitude of gravitational acceleration by cos(angle of tilt about y-axis) before proceeding with the calculations to determine the angle of tilt, again as described previously, e.g.:

arcsin{[(measured gravitational acceleration in y-axis accelerometer)/g]/[cos(angle of tilt about y-axis)]}=“angle of tilt about x-axis”

The angle of tilt about the y-axis can be measured by other means as well. The tilting about the y-axis is often related to the tilting of the support surface. This tilting of the support surface can be determined by placing or attaching a separate orientation sensor in a fixed position relative to the support surface and determining the orientation of the support surface, or part of the support surface. This can also be achieved by having information regarding the orientation of the support surface entered into the system. This data collection can either be done manually or by directly communicating with the support surface (if the support surface has orientation sensors and has the ability to output the data in a usable format).

In some embodiments it is desirable to calibrate the accelerometers to achieve a desired accuracy. Calibration determines constants that enable acceleration to be described in real, physical units. During “calibration”, the device's raw output can be calibrated by determining the appropriate constants that can be used to determine physical units such as m/s/s, ft/s/s, g's, etc. The calibration process can involve determining the readings from the accelerometers throughout a representative sample of its orientations. Calibration constants can be determined and used to get more accurate acceleration data. One method of determining the calibration constants is to orient the sensor such that it experiences 1 g and −1 g of acceleration along each of the axes in which acceleration is measured. The sensor can then be calibrated such that the output from the accelerometer when it experiences 1 g or −1 g of acceleration is associated with a 1 g or −1 g acceleration, respectively, This process can be done prior to distributing the sensor to end users, or it can be performed by the end user using instructions or calibration tools that can be provided. The calibration constants can provide, for example, multipliers and offsets such that a calibration equation may be acceleration=(accelerometer reading)*M+0, where M is the multiplier and O is the offset. Depending on the degree of linearity of the accelerometer readings throughout its range, the calibration equation can take on forms other than that of a linear equation.

In addition to calibrating the accelerometers, it is also helpful in at least some embodiments to calibrate the angle of the accelerometers with respect to the rotational axis of the MP on whom the sensor will be placed. A typical placement of the sensor is on the sternum. However, for most people, the sternum is not perfectly parallel to the rotational axis of the body, which basically runs vertically from the center of the skull down to the feet. Instead, for most people there is a slope downward from the sternum to the neck, and this downward chest angle, or pitch, can vary significantly, perhaps as much as −50 degrees or more although 30 degrees is more typical, measured with respect to a line parallel to the rotational axis and tangent to the sternum.

Thus, to improve the accuracy of the sensor in detecting the rotation of the user, a correction factor or offset equal to the opposite of the MP's downward chest angle can be applied by applying a rotation matrix to rotate, about the Y-axis, the gravity vector detected by the accelerometer by the appropriate offset. Expressed mathematically, the correction is:

$${R}_{y} ( θ ) = [ \begin{matrix}cos ( θ ) & 0 & sin ( θ ) \\ 0 & 1 & 0 \\ - sin ( θ ) & 0 & cos ( θ )\end{matrix} ]$$

$${R}_{y} ( θ ) = [ \begin{matrix}ngx \\ ngy \\ ngz\end{matrix} ] = [ \begin{matrix}ngx cos ( θ ) + ngz sin ( θ ) \\ ngy \\ ngz cos ( θ ) - ngx sin ( θ )\end{matrix} ]$$

For a correction of angle θ (i.e., the offset for a downward slope of −θ), the process performed in the processor comprises: the cosine θ and sine θ are calculated; raw acceleration values are collected from the accelerometer; the acceleration values are normalized to give ngx, ngy and ngz, and the rotation matrix shown above is applied. With the now-corrected values in hand, the remainder of the process of determining the user's orientation, or change in orientation, continues in the normal manner. It will be appreciated by those skilled in the art that the normalization can occur before or after the rotation. In some embodiments, it is adequate to apply a fixed correction offset for all MPs. In embodiments which utilize such a global offset, it can be beneficial to choose an offset value conservatively, i.e., smaller, since some chests have shallower angles, or even a positive angle. In a more generally applicable embodiment, it can be desirable to determine a correction offset based on the actual chest angle of the specific MP or other user. This can be done by placing the MP in a known orientation, for example either supine or standing vertically, such as against a wall. The accelerometer measurements from the sensor are then taken, and the chest angle for that specific MP can be calculated from those measurements with reference to the known orientation.

An embodiment of the process described above can be appreciated from FIG. 4B. The process starts at 420, with the decision that a correction is to be applied. If an MP-specific correction angle is available, as discussed above, the process advances to step 425 where that correction value is retrieved from its storage location, typically expressed in degrees or radians and indicated by θ. If a global correction value is to be used, the process advances from step 420 to step 430 where the global correction value is retrieved from its storage location. In either event, the sine and cosine of the correction value θ are calculated at 435. The uncorrected, or raw, acceleration values are also retrieved either from the accelerometer (and associated processing as appropriate for signal compatibility) or from a storage location, shown at 440. In the exemplary embodiment illustrated in FIG. 4B, the acceleration values are then normalized in step 445, and a rotation matrix is applied at step 450. As noted above, normalization can be performed either before or after the application of the rotation matrix. In either approach, the normalized output of the rotation matrix step is provided to the remainder of the process for determining orientation, as shown at 455.

In some instances, the sensor is not placed on or near the sternum, and instead is placed laterally of the MP's midline, and closer to the clavicle. In such instances, a roll correction may be desirable in addition to the pitch correct discussed above. The calculations for correction of roll are analogous to those described above, although the rotation matrix is applied around the X-axis. Further, in the event that the sensor is not placed on the MP in accordance with indicia on the sensor, a yaw correction may also be desirable. In an embodiment, correction for yaw can be achieved by the combination of an accelerometer and a magnetometer, with the magnetometer calibrated to magnetic north or providing a reference magnet in a known and repeatable location, such as the at the MP's head or foot or aligned with the MP's longitudinal body axis.

An accurate alignment of the sensor with respect to one or more of the rotational axes of the body is also relevant in the instance where an MP either should not be placed in a specific position, such as in an orientation that would put undue pressure on an existing wound, or in instances where an MP should be maintained in a particular position. In the latter instance, for example, an MP suffering from or at risk of developing pneumonia can be subject to a head-of-bed (“HOB”) elevation protocol. MPs at risk of gastric reflux into the lungs are one exemplary group subject to head-of-bed elevation requirements to avoid, or resolve, Hospital-Acquired-Pneumonia (“HAP”). MPs receiving mechanical ventilation or tube feedings are another, although there can be overlap between those groups. MPs on ventilators can be at risk of Ventilator-Acquired-Pneumonia (“VAP”). A head-of-MP elevation protocol, used to address both HAP and VAP, typically proposes that the head of the bed be elevated by 30 or more degrees, although in some instances the recommendation is 45 degrees or more. This, in turn, is expected to elevate the head of the MP to a corresponding angle. While many beds include alarms, the position of the MP torso on the bed's support surface can vary if, for example, the MP slides down the elevated portion of the bed's support surface. Historically, the elevation angle of the MP has been inferred from the elevation angle of the head of the bed, whereas the more accurate assessment is the angle of elevation directly from the MP, not the bed. Thus, a more accurate term for the HOB protocol might be Head-of-MP [“HOP”] to better reflect that the measurement of interest is the angle of elevation of the MP's head and torso, and HOP is used hereinafter to reflect an HOB protocol but based on a measurement of MP elevation angle rather than an inference of MP elevation angle from the angle of the head of the bed.

Some embodiments of the system described herein can serve to provide position-optimizing to confirm compliance with a HOP protocol. Some embodiments are configured to detect the MP's actual position, and confirm that torso of the MP is elevated in accordance with the elevation protocol. As discussed above, the accelerometers automatically determine the position and orientation of the MP, including an elevation of the torso, and, instead of or in addition to the checks made as shown in FIG. 2, a check is made for the elevation angle of the torso. If the MP's HOP angle falls outside of defined thresholds, for example less than 30 degrees or some other value deemed appropriate by the caregiver, the position-optimizing system provides one or more alerts to care providers. The alert can be configured to be issued either immediately when a position threshold is violated, or after a certain amount of time has elapsed in a sub-optimal position. In addition, the timing of an alert notification can be based on the magnitude of the position violation. For example, if an MP exceeds positioning parameters by only one degree, an alert notification might not be issued either immediately or at all, or might be issued after an extended period of time in order to give the MP an opportunity to return to the correct position. Allowing the MP an opportunity to change without issuing an alert can be helpful in avoiding alarm fatigue. However, if an MP varies from the expected elevation parameters by, for example, 20 degrees, the severity of the position violation is such that an alert may be issued either immediately or after a shorter period of time than for lesser violations. The assessment of a specific MP's condition, performed either automatically or by a caregiver, can be used to assess the level of need for compliance, or the need for compliance can be based on predetermined characteristics.

Importantly, there are sometimes conflicting positioning goals between HAP/VAP prevention and HAPU prevention. In contrast to HAP/VAP guidelines, pressure ulcer guidelines suggest keeping the head-of-bed angle at less than 30° to avoid excessive pressure on the sacral region. For an MP at risk for both HAP/VAP and HAPUs, balancing of the conflicting objectives can result in the optimal position being the lower end of the range of acceptable elevations for a HOP protocol. For example, a compromise between a pressure ulcer turn protocol and a HOP protocol might be at exactly 30 degrees.

In addition to providing alerts if the HOP (i.e. “Head of MP”) angle is outside of a defined threshold, the position optimizing system can intelligently optimize repositioning schedules as a function of HOP angle. For example, as the HOP angle increases, the turn interval can automatically decrease, and vice versa. In addition to altering turn intervals as a function of HOP angle, all of the turning parameters can be automatically modulated as the HOP angle changes. For example, lateral turn angles can be increased/decreased, tissue depressurization time can be increased/decreased, and other parameters can be adjusted to help optimize lateral rotation based on upright angle.

Calibration of the accelerometers throughout their desired range of orientations can allow for more accurate orientation measurement. Each type of accelerometer, or each individual accelerometer, can be tested and calibrated depending on the level of accuracy desired. A plot of the angles calculated based on the accelerometer data vs. the actual angle being measured can be used to create a regression that can then be used to improve the accuracy of the calculation. Once the regression is made, the calculation of orientation can be made using data from the regression. The physiologic heart rate has a range, speaking generously, of approximately 30 to 350 bpm. So when isolating the accelerative signal from the heart beat, one can choose to look at signals within this range or a similar frequency range. A band pass filter can be used to attenuate signals with frequencies above and below this range. Since the accelerative forces due to the heart beat can be large relative to the other accelerative forces experienced in a resting user, there may not be a need to significantly filter the data in order to detect the heart rate with reasonable accuracy. The heart rate can be detected by looking for periodic signals that have a higher than normal amplitude or a low-pass filter can be used to attenuate signals above a certain frequency. For example, frequencies higher than approximately 6 Hz (i.e. 350 bpm) can be attenuated. It is also possible to increase or reduce the amount of filtering, by changing the attenuation or changing (shifting, narrowing, broadening, etc.) the band pass frequencies. For instance, for a resting MP, the range of frequencies that are most common may be 35-120 bpm. A band pass filter covering this range may be useful for most cases. It is possible to capture other frequencies by having a separate, wider, or shifted filter that is added on with a different gain or analyzed separately to accommodate for less common heart rates. The attenuation can also be turned down to similarly increase the range of frequencies. A tight band pass filter (e.g. covering a narrower range of frequencies or having greater attenuation, etc.) can provide cleaner signals; for example, a Butterworth filter can be used, although many other types of filters can also be used.

The quality of the filtering becomes more important when the signal is smaller or when there is more noise. Some examples of when this can occur include: the sensor is not placed close to the heart (e.g. in the pelvic area), when the user has more material intervening between the sensor and the heart or artery (e.g. skin, fat, non-organic materials like clothing, etc.), or when the pulse is weaker (e.g. impaired heart contraction or low blood pressure/pulse pressure). In such cases the filtering becomes more important and the methods described above for improving the filter may be required to isolate the heart rate. The optimal placement of the sensor is in close proximity to the heart (or major arteries) in order improve detection of the pulse and heart rate. Placing the sensor on the chest, especially near the sternum, is optimal for detecting the heart rate. Placing the sensor at locations close to the aorta or other large arteries are good sensor placements for detecting the heart rate at locations more distant from the heart.

When the sensor is placed with the 3 axes oriented as shown in FIG. 5A, the heart rate (and breathing rate), is sensed mainly by the z-axis accelerometer. When positioning a sensor that is intended to detect heart rate or breathing rate, the quality of the signal is improved if at least one accelerator is positioned in approximately the anterior-posterior axis (or z-axis as shown above).

In certain cases it can be useful to keep track of when:
the heart rate (HR) is above a certain threshold
the HR is below a certain threshold
when the HR changes quickly
when the HR is irregular
when the magnitude of acceleration is above or below a certain threshold
when the magnitude of acceleration changes quickly or is at a rate above a certain threshold
when the heart rate detected by the accelerative sensors is different from the heart rate detected by electrical signal sensors.

This can be important for cases of ventricular fibrillation, where electrical signals from the heart are present but the mechanical heart beat is not present or is irregular. In such a case, the accelerometer data can be compared with EKG data, where the signal detectors for EKG data are either external or internal to sensor 300.

Detecting the mechanical activity and/or electrical activity of the heart, as described above, can provide an indication of abnormal physiologic conditions, such as tachycardia, bradycardia, arrhythmias, heart attacks, pulseless electrical activity (PEA), heart failure, etc.

It may also be desirable to sense an MP's breathing rate (respiratory rate) in some embodiments. The physiologic breathing rate has a range, speaking generously, of approximately 3 to 100 bpm. When isolating the accelerative signals resulting from breathing, it is desirable for at least some embodiments to choose to look at signals within this or within a similar frequency range. As with heart rate, a band pass filter can be used to attenuate signals with frequencies above and below this range. Often the accelerative forces that result from breathing, especially with breathing at rest, are small relative to the heart rate. As such, filtering can be desirable. Methods to improve the filtering beyond a basic band pass filter can be implemented if appropriate to the embodiment. This includes, for example, narrowing the band to between 5-30 breaths per minute. A band pass filter covering this range may be useful for most situations. Another issue is that the range for physiologic breathing and heart rate can overlap. One can take advantage of the fact that the heart rate is usually higher than the breathing rate. The narrowed band pass filter can achieve the desired differentiation. The filtering can also be adaptive, such that the heart rate is detected first and then the filter adjusts so as to have an upper cutoff that is below the heart rate. As with heart rate, a tighter band pass filter can yield cleaner signals; again a Butterworth filter can be used, among a variety of acceptable band pass filters.

In certain cases it may be useful to keep track of an MP's breathing rate, such as when:
the breathing rate (BR) is above a certain threshold
the BR is below a certain threshold
when it changes quickly
when it is associated with the administration of medications
when it is irregular
when the magnitude of acceleration is above or below a certain threshold
when the magnitude of acceleration changes quickly or at a rate above a certain threshold
when the heart rate is below the breathing rate
certain patterns of breathing, e.g. Cheyne-Stokes respirations

Detecting the respiratory rate and breathing pattern, as described above, can provide an indication of abnormal physiologic conditions, such as tachypnea, hypoventilation, Cheyne-Stokes (strokes, brain injury, encephalopathy, heart failure), etc.

MPs may move on their own, and it can be useful to determine their activity level or lack thereof. It is important to isolate this signal from other physiologic signals. In general, there are components of acceleration that are due to the normal voluntary movements of a user. These movements can have a magnitude that is greater than the acceleration due to breathing, heartbeats, and pulses. One method of isolating a user's movement-based acceleration is to isolate the accelerations that have magnitudes beyond those expected to be due to breathing and heart beat/pulses. This threshold of magnitude can be pre-programmed based on physiologically normal accelerations due to heartbeats, pulses, and breathing. The threshold can also be directly measured from the accelerations measured on the user, either at the same time or during another time when the MP is determined to be still. Another method of isolating movement-based accelerative signals is to subtract the filtered signals of the heart rate and breathing from the initial signal.

The user may be subjected to environmental noise, such as due to machinery. Many MPs that are at risk for pressure ulcers are put on “alternating-pressure” mattresses. These mattresses have a series of individual air columns that independently modulate their pressure, thereby creating depressurization waves that travel under the MP. Although these waves can travel very slowly, they can cause subtle movements of the MP that will need to be accounted for. Algorithms for filtering out this noise, as well as any other environmental noise, will be straightforward for those skilled in the art, given the teachings herein. Environmental noise can also be due to electrical interference, etc. Undesirable environmental noise sources may include nearby electrical or mechanical equipment, building HVAC or other infrastructure systems, and/or other human activity.

The movement-based accelerative sensing can be used to monitor the activity level of the MP in order to encourage activity or to discourage activity. It can also be used to automatically determine mobility for the purposes of charting, for example for determining some of the components of the Braden scale (i.e. mobility, activity, shear forces, etc.).

Other signal analysis can be performed on the signals from the accelerometers. The overall waveform of acceleration due to heart beat/pulse is known, as well as the waveform for the accelerations involved in breathing. Signal analysis can be used to analyze the waveform of accelerative signals to gain more information from the signal, such as its source or association with different physiologic conditions. For example, the waveform for a breath is different from the waveform for a heart beat or pulse. Thus, the waveform and/or the frequency can be used to help isolate/identify the HR and BR. The waveform of an MP turn can also be identified. In addition, within the accelerative waveform of the heart beat/pulse, there can be different physiologic conditions that affect the waveform. For instance, a different waveform exists between normal heart beats and ventricular fibrillation. Changes in waveform or abnormal waveforms can be detected in this way. This applies similarly for breathing. The algorithms can also learn from the normal state of the user to help better identify the range of normal HR and BR as well as the normal waveforms for a particular user. This will be useful when any of these change greatly. This algorithm can also learn from greater data sets from one user or multiple users to improve its accuracy and precision.

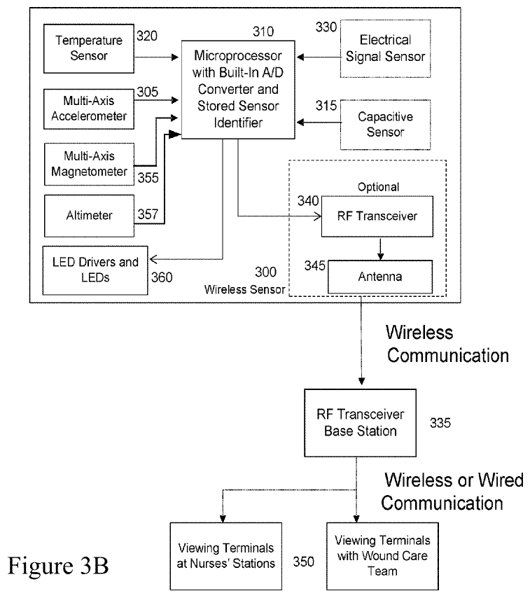

FIG. 7 illustrates the foregoing process, and shows the filters used to isolate orientation, heart rate, breathing rate, and movement data from the initial accelerometer signals, as well as paths to enable the filters to learn. More specifically, in an embodiment, signals 700 from the accelerometers are received by a set of four parallel filters 705-720, including a filter 705 for isolating gravitational accelerative forces, a filter 710 for isolating heartbeat/pulse accelerative forces, a filter 715 for isolating breathing accelerative forces, and a filter 720 for isolating accelerative forces due to movement. In addition to movement and orientation, the acceleration measurements can be used to detect other characteristic accelerative events, such as falls. At block 725, the heart rate output is used to provide an upper cutoff for the breathing rate filter, and feeds from block 725 to filter 715. Likewise, heart rate and breathing rate can be subtracted to isolate movement, as shown by block 730 feeding to filter 720.

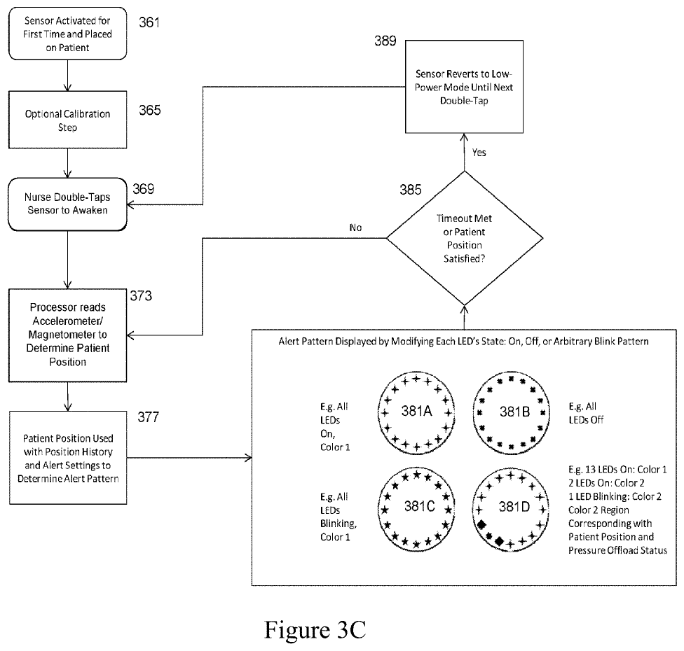

Referring next to FIG. 8, one of the filters of FIG. 7 can be understood in greater detail. The accelerometer signal 800 is provided to low pass filter 805, with a cutoff below the minimum physiological breathing rate, to isolate orientation as shown at 810. To isolate heart rate, the signal 800 is fed to a band pass filter 815 with a physiological range of heart rates, or a subset, as the cutoff, yielding an output of heart rate as shown at 820. In addition, heart rate data is fed via block 825 to a band pass filter 830, which also receives the signal 800 and isolates breathing rate as shown at 835, including using heart rate as the upper cut-off for breathing rate. Amplitude threshold block 840 also receives the signal 800, and isolates activity and mobility level as shown at block 845.

There are instances when an MP's vital signs can be affected by positional changes. The position/orientation sensors described herein can be correlated with an MP's vital signs in real-time. Data from the position/orientation sensors can be correlated with vital sign measurements that are obtained via standard modalities (EKG, blood pressure cuff, manually counting palpable pulsations of the arterial pulse, manually counting respirations, etc.). Data from the position/orientation sensors can also be correlated with vital signs using a single sensor that can determine both the MP's position/orientation and vital signs. In one implementation, an accelerometer placed on the MP can determine the position/orientation of an MP, as well as the heart rate and respiratory rate. When the sensing system detects dramatic changes in heart rate that are associated with changes in position/orientation, caregivers can be notified that the MP may have orthostatic hypotension. MPs with orthostatic hypotension will commonly experience a decrease in blood pressure upon standing that is associated with a rapid acceleration in heart rate (usually an increase of over 20 bpm). In fact, the diagnosis of many conditions (i.e. orthostatic hypotension, autonomic dysfunction, postural orthostatic tachycardia syndrome, etc.) can be aided by using a tilt-table test, where MPs are put on a platform that tilts and vital signs are monitored.

There are other instances when an MP's vital signs are affected by position. For example, when MPs with CHF lie flat they can develop respiratory distress that manifests as an increased respiratory rate. Similarly, MPs with morbid obesity or obstructive sleep apnea can develop respiratory distress when they lie flat (the extra weight due to fat around the chest and neck can increase the work of breathing) and these MP's breathing patterns can change based on the postural changes. In one implementation, an accelerometer placed on the MP can measure both the MP's position/orientation and respiratory rate. When the sensing system detects changes in respiratory rate that are associated with changes in position/orientation, caregivers can be notified and further workup initiated.

Conditions that can be affected by position can be entered into the monitoring system. For example, if a particular MP has CHF resulting in severe orthopnea, this condition is entered into the system and then the turning recommendations allow for the MP's head/chest to remain elevated by 30 degrees throughout the day (MPs with CHF can't handle the extra fluid load that occurs when lying supine, hence they get short of breath when lying flat). As a consequence, since the MP's head/chest is elevated throughout the day (thereby increasing the pressure-dose on the sacrum), the system can then recommend increasing turning frequencies, etc. to help prevent sacral ulceration. Any condition of the MP (i.e. paralysis, amputations, injuries, diabetes, anorexia, obesity, etc.) can be defined in the system.

It has previously been described herein how sensing the MP's breathing pattern and heart rate can be used to determine if the sensor is properly affixed to the MP.

Similarly, electrodes or capacitive sensors which are capable of measuring the body's electrical activity, impedance, or resistance can be used to determine if the sensor is properly affixed to an MP. A thermometer can also be used to determine if the sensor is properly secured to the MP. When the skin surface temperature reading shows temperatures sufficiently close to the expected skin temperatures, it can be assumed that the sensor is affixed to the MP. Similarly, if a sudden change in the skin surface temperature is detected, it can be inferred that the sensor has lost continuity with the MP.

Another technique that can be used to determine if the sensor unit is properly attached to the MP is a tab that is attached to a conductor within the sensing unit's circuitry. After the unit is affixed to the MP, if the unit is subsequently removed, the tab detaches and changes the circuit in a measurable way, such as by changing the resistance. This allows the sensing unit to know that it has been removed from the MP and the sensing unit can send this information to the host or other reader. In some arrangements, the tab can also be affixed with greater strength to the MP due to differences in affixing compound or a heat-activated bonding substance.

The sensor unit can be oriented to work automatically when placed anywhere on the MP. In this care, orient means to determine the direction of the accelerometer with respect to gravity or with respect to the MP. During “orientation”, the accelerometer's direction can be determined with respect to gravity by measuring the acceleration in the three axes as the device is rotated in each of the three axis of rotation. Some placements can be at the sternal notch or the xiphoid process of the sternum or the anterior superior iliac spine (ASIS). The sensor unit can also be placed anywhere on the MP and oriented to the MP. In an embodiment using this approach, the MP lies supine with the sensor unit in place. A button on the reader unit, the sensor itself, a remote, or a computer interface can be pushed or a command sent once the MP is supine, and the reader unit will then associate the reading from the sensor unit with the supine position. Thus, the sensor unit can be at placed at any angle relative to the MP and the system will be able to oriented accordingly. The signal to the system that the MP is supine can come in any number of forms including voice activation, etc.

Different sensors can be pre-calibrated for use on MPs with different body types. For example, a sensor that has a unique identifier can be placed on MPs that have a specific BMI. In such a manner, the system will detect the unique identifier from the sensor, and automatically calibrate the monitoring system for an MP with a specific BMI.

Similarly, the sensors placed on the support surface can be pre-calibrated for use on support surfaces with different properties. For example, a sensor that has a unique identifier can be placed on support surfaces that have a specific surface pressure profile (i.e. dry pressure, air pressure, air fluidized, etc.). In such a manner, the system will detect the unique identifier from the sensor, and automatically calibrate the monitoring system for a support surface with a specific surface pressure profile.

In at least some embodiments, the sensing system is designed such that it does not require any additional manipulation by a care provider. As previously described, the sensor can automatically be activated when its adhesive backing is removed. The removal of the adhesive backing allows for the activation of a sensor circuit and hence discharge of the unit's on-board battery. To conserve power, the sensor can locally store acceleration data and transmit this information to the receiving station(s) at predefined intervals. A disposable sensor unit can be designed such that it is able to transmit acceleration data for an extended period of time, such as days or weeks.

The sensor unit can be designed such that it does not draw power (or at least very little power) when it is in its packaging. In some embodiments, it is activated immediately before being placed on the MP. Alternatively, a signal received from the transceiver can serve to activate the sensor unit. One type of activation signal can be an RF signal that is sent to the unit. If the sensor unit is not a passive RF unit, the unit can temporarily act as a passive tag before activation and be powered by the received signal. As another alternative, a passive tag or an RF receiver/transceiver that has the ability to passively receive signals can be initially included as part of the sensor, and can be used to allow for a signal to be received by the by the sensor without using stored energy in the sensor. This signal can be used to activate the sensor. The passive tag can then be removed promptly following activation, as a method for reducing the size of the sensing unit and allowing the passive receiver/transceiver antenna to be larger.

For units that sense physiologic variables such respiratory rate, heart rate, and/or temperature, in an embodiment the reader can allow for a period of time (seconds, minutes, or hours) after activation before it expects physiologic values to be measured. This can allow time to attach the sensor to the MP before the system expects to receive physiologic data.

Another variation has the sensor activated by a switch on the unit.

Proper placement of the sensor 300 on the MP is important in at least some embodiments. In at least some embodiments, the sensor is placed on the MP such that there is no potential for movement of the sensor with respect to the MP. In an embodiment, the sensor is adhered directly to the skin using an adhesive patch, which can be similar to that used for standard EKG leads, although in other instances the sensor can be removed from the adhesive backing to permit replacement of the sensor while protecting the MP's skin.

The sensor is ideally placed on the anterior thorax, pelvis, upper thigh or shoulder. In ideal usage there is little relative movement between the sensor and the user's pelvis, which enables an approximate determination of the orientation of the user's pelvis. In an embodiment, the sensor must be placed at a location on the body where the orientation of the sensor approximates the orientation of the MP's pelvis and/or thorax.

By knowing the orientation of the MP's pelvis and/or thorax, the surface pressure distribution across other body structures can be estimated. For example, if it is determined that an MP is in a completely supine orientation, it is then known that surface pressure is being exerted on the MP's sacrum, and ischium. However, based on the MP's orientation and the known anatomic relationships that exist between different body structures, it can be inferred that structures such as the posterior occiput, elbows and heels are also experiencing pressure. If the MP then turns to a left lateral decubitus position, it can be determined that surface pressure has been transferred to the MP's left hip, as well as other body structures, such as the left shoulder, left elbow, left occiput, and left lateral malleolus.

When the MP's pelvis is determined to be in a left lateral decubitus position, it is very unlikely (if not impossible) for surface pressure to be exerted on the MP's right hip, right occiput, right elbow, right shoulder, or right lateral malleolus. There are anatomic relationships that exist between different body parts that prevent pressure from being exerted at these locations. In such a fashion, the overall surface pressure distribution map of an MP can be estimated based on the known orientation of one or more body structures, such as the pelvis or thorax.

In at least some embodiments, it is preferred that the sensor not be placed on the limbs or head, because the orientation of the limbs does not always approximate the orientation of the pelvis/thorax. The location of placement may be different if the primary concern is for preventing and managing pressure ulcers at locations other than the pelvic region. For example, if the MP has a pressure ulcer on their right heel, a sensor can be placed on or near the right foot, ankle, or lower leg to better approximate and monitor the orientation and surface pressure distribution of the affected region. In an embodiment, the sensor should not be placed in a location where it will be susceptible to being rolled on.

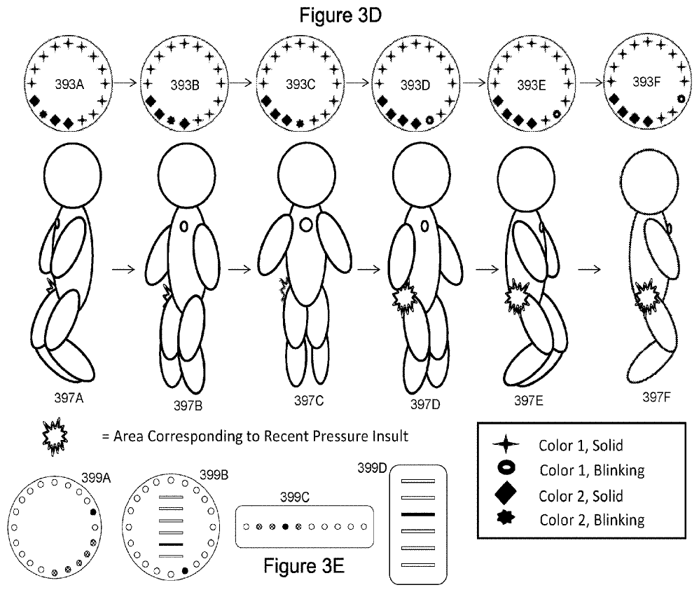

In order to accurately determine a user's orientation, it is important in at least some embodiments to know the orientation of the sensor with respect to the MP. To facilitate properly orientating the sensor with respect to the MP without requiring significant training, an index mark can be provided on the sensor 300. Such index marks can provide information including but not limited to which direction the sensor should be oriented (e.g., top of sensor towards the MP's head) or where on the MP the sensor should be placed. Examples of index marks are shown on the different sensors 900A-900E illustrated in FIG. 9, including two, 900C-D, with cross-hairs on a representation of a human for indicating the location where the sensor should be placed and where the orientation of the human image on the sensor is to be aligned with the user (i.e., head pointing in same direction in image and user). The three other examples in FIG. 9 are for indicating simply the desired orientation of the sensor including an arrow, an arrow labeled “head”, and a human image representation. In an embodiment, the orientation of the sensor with respect to the MP must be determined to accurately determine the relative surface pressure distribution of the MP. The indicia need not reference the head, as long as there are sufficient and simple instructions or indicia to place the sensor relative to an identifiable landmark on the body and a in a relative orientation to that landmark, whether the landmark be the sternum, belly button, anterior superior iliac spine (ASIS) spine, leg or other. The indicia can include but are not limited to markings on the sensor, the shape of the sensor itself, different materials or colors used on different parts of the sensor, or asymmetry of the sensor. The shape of the sensor or adhesive backing can also be more suitable to fitting in or conforming to specific areas of the body in specific orientations. The sensor can be incorporated into articles that may be worn by the MP such that when the article is worn the sensor is in an appropriate location and orientation.

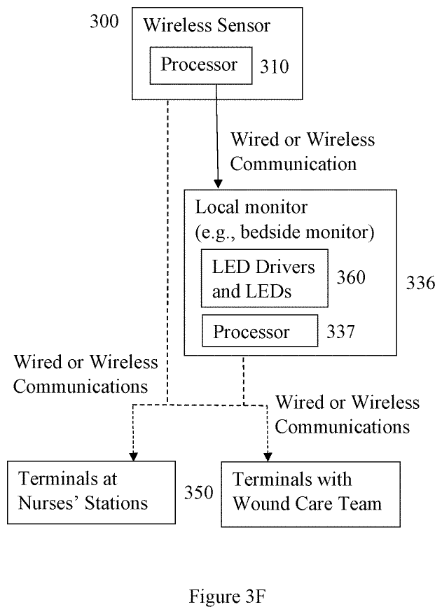

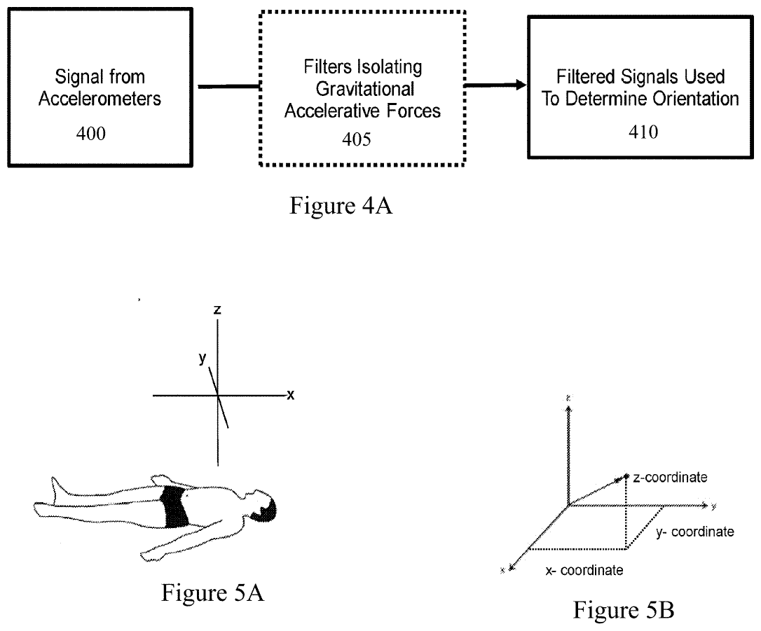

In an embodiment, it is possible to automatically determine the orientation of the sensor 300 by, for example, sensing bioelectrical signals in the body. It is well understood that electrical impulses propagate away from the heart in a well-defined pattern, and the body has a known and well-defined polarity that can be detected. Referring next to FIG. 10, by providing the sensor 1000 with multiple bioelectrical sensors 1005 positioned circumferentially around the outer surface of the sensor, the plurality of bioelectrical sensors can be used to detect the average direction of electrical propagation, and either the sensor itself or the remote host can process the data to identify the orientation of the sensor 1000 with respect to the heart. In such an arrangement, the sensor 1000 can be placed on the MP at virtually any location on the thorax/pelvis (and in any orientation) and the sensor can automatically determine its orientation with respect to the MP.

Referring still to FIG. 10, the sensor picks up the electrical signal between the electrodes shown as open circles in reference to the common electrode 1010 shown as a solid circle and depending on the vector of the body's electrical signal at the location of the sensor can determine its orientation with respect to the MP. The magnitude of the signal (which can be an average or integrated magnitude) from the different electrodes gives an indication of the direction of the vector. For instance, if the signal from one of the electrodes shows a greater magnitude than the rest, then the vector can be determined to be closest to the direction of the line intersecting that electrode and the common electrode. The vector can also be determined to be in the direction between the two electrodes with the greatest magnitude of detected signals. As the signal detected in the electrodes can be positive or negative, the plurality of electrodes need only span approximately 180 degrees, for example, roughly a semicircle, to determine the vector direction within a 360 degree range, thereby reducing the number of electrodes needed per device and the number of sensing inputs and/or A/D converter inputs.

In some embodiments the monitoring system can track, record, and display relative surface pressure distribution data for an MP and alert caregivers when it's indicated to reposition an MP. Since the orientation sensor is placed in a known orientation relative to the MP (using visual indices and auto-orientation mechanisms), the system has the ability to know when pressure is being exerted on specific areas of their body. The system can also determine the cumulative amount of time that pressure has been exerted on specific areas of the body, and thereby calculate the pressure dose for specific areas of the body. The system can monitor the pressure dose at specific areas of an MP's body, and use this information to determine an MP's requirement for repositioning. The system can use this information to help ensure that MPs are turned as often as necessary, but not more often than necessary. In addition, the system can suggest the optimal direction to reposition an MP by analyzing the pressure dose at specific areas of the MP's body and suggesting repositioning maneuvers that allow for the MP to be preferentially positioned onto regions of the body that have a low pressure dose.

Still further, the system that can automatically detect when an MP initiates a turn by themselves or if a turn is initiated by a care-giver. In an embodiment of this aspect, an RFID tag on the caregivers badge configured to be recognized by the orientation sensor on the MP or by the base station residing near the MP. When the two (ID badge and MP sensor) come in close proximity with each other, and the system subsequently detects an MP turn, it can be noted that the turn was performed when a caregiver was present. Other methods for doing this include having a button on the sensor or user interface that is pressed to indicate a care-giver turn was performed; still others will be apparent to those skilled in the art, given the teachings herein. This information can be helpful, as it may be a factor that helps indicate when an MP is sufficiently mobile, and thus no longer requires continued monitoring and caregiver assistance. However, if an MP is determined to not be moving sufficiently on their own, it may indicate that this MP requires continued monitoring and caregiver assistance.

In some embodiments, the system not only keeps track of how long a user has been exerting pressure on specific areas of their body, but also keeps track of how much time specific areas of the body have had to depressurize. This is important because sufficient blood flow to a tissue (where it is free of pressure above a threshold that restricts blood flow), is required for a sufficiently long period of time in order to resupply said tissue with oxygen and vital nutrients. This is referred to as the re-perfusion interval. The desired re-perfusion interval can be set by the user, by caregivers, or can be taken from a protocol. The re-perfusion interval may also vary depending on the MP. For example, an MP's co-morbidities, Braden score, nutrition status, past history of pressure sores, or feedback from perfusion sensors can be used to determine an appropriate re-perfusion interval.

Knowing the MP's orientation relative to the support surface can be important for pressure ulcer management. When information regarding both the orientation of the support surface relative to gravity and the orientation of the MP relative to gravity is provided, the system can determine the relative normal force of the support surface (pressure) as well as the tangential force of the support surface (shear force).

At different angles of MP rotation relative to the support surface and to gravity, the MP experiences pressure on different portions of their body. This is the basis for the turning protocols, which allows for periodic de-pressurization of areas of the body in sequence. The system can determine, from the orientation of the MP relative to the support surface and to gravity, which areas of the body are experiencing pressure, and thereby creates an orientation-based pressure distribution model of the MP. The system can also keep track of how long the MP is in any given position and thus how long certain areas of the body are experiencing significant pressure. As the MP is repositioned, the system can monitor the angle of MP rotation, and determine if there was a sufficient change in an MP's orientation, so as to provide a threshold level of depressurization at specific areas of the MP's body. For example, if the MP is insufficiently rotated, certain areas of the body may not experience de-pressurization. The system can monitor and track the pressure at different body regions using the orientation-based pressure distribution model. The system can determine when certain body regions require de-pressurization, and thus indicate that a change in MP orientation is required. In such a fashion, the system can optimize a turning schedule and ensure that MPs are turned as often as necessary, but not more often than necessary. The system can also ensure that MPs are turned with sufficient frequency and with sufficient de-pressurization intervals so as to provide sufficient time for tissue perfusion.

Additional sensing elements for detecting other physiologic characteristics can be attached to or incorporated within the sensor 300 in addition to the one or more accelerometers and RF units previously described. One such sensor is a pedometer. This can be used to track the number of steps an MP takes or the amount of movement he/she engages in. The data from the pedometer can be sent in conjunction with the data from the accelerometer. As previously mentioned, electrical leads can be incorporated to monitor the heart or other muscle activity. Likewise, capacitive sensors or piezo-electric sensors can be incorporated to detect heart sounds, breathing sounds, or other vibrations. Similarly, a pulse oximeter can be incorporated to provide oxygenation data, and a temperature sensor can provide temperature monitoring.

Since the sensor 300 is, in at least some embodiments, powered by a battery or similar device, it is desirable in some embodiments to conserve power. Some embodiments provide power management, including burst data transmission, either at regular intervals or in response to a predefined trigger. Portions of the sensor can be powered down when not needed, including the transceiver, microprocessor, sensors, etc. In an embodiment, the sensors can be used for a period, then powered down, and still successfully monitor heart rate and breathing. Capacitive and temperature sensors in some embodiments may need only one reading between power downs.

Low power states can be indicated in a variety of ways, including flashing, varying intensity on a display, different response when interrogated, and transmission of battery information or “I'm alive” information.

As discussed previously, in some embodiments it can be desirable to be able to remove the sensor from the backing affixed to the MP. In such circumstances, it is desirable both to ensure that the orientation relative to the MP is maintained, and also to ensure that the new sensor is secure, an asymmetric relationship between the backing and the sensor can be used, together with any suitable locking mechanism. In other embodiments, the relationship between the sensor and backing may not be fixed, but automatically sensed indicators such as electrodes, reflective patches, etc., can be used to inform the system of the new relative position.

Ultrasound can be used in some embodiments as a sensing modality to gather physiologic data from the user. This data can be used alone, or in combination with other sensing modalities, to assess the perfusion status of an MP at discrete locations on their body. Doppler ultrasound can also be used to assess blood flow. If areas of abnormal perfusion are detected, the support system can automatically optimize surface interface pressure at those locations, and caregivers can be alerted. Pressure optimizing maneuvers performed by the support system can be used to promote blood flow to critical areas.

In some embodiments, tissue oxygen tension, carbon dioxide tension, pH and hydration status can be analyzed remotely using near-infrared optical spectroscopy. The skin is a relatively weak absorber of near-infrared light, so near-infrared spectroscopy can be used to analyze the epidermis and dermis. Near-infrared spectroscopy can be used to examine spatial and temporal changes in tissue hemodynamics and can provide pre-clinical detection of perfusion abnormalities. When perfusion abnormalities are detected, the support system can automatically redistribute pressure away from areas of compromised tissue perfusion.

Hemoglobin has distinct absorption bands in the near-infrared spectrum, depending on whether the heme group is oxygenated or deoxygenated. When tissue is exposed to near-infrared light, the chromophores within the tissue (such as oxygenated and deoxygenated hemoglobin) will absorb light at distinct wavelengths. Thus, the light that is ultimately reflected off of the tissue will contain wavelengths of light that were not absorbed by the chromophores. Oxygenated hemoglobin absorbs near-infrared light strongly in the 900-950 nm range, while deoxygenated hemoglobin absorbs near-infrared light strongly in the 650-750 nm range.

Water is the major component in tissue, and it absorbs near-infrared light most strongly at wavelengths above 900 nm. The absorption characteristics of water are distinct from hemoglobin, so water can be analyzed independently of hemoglobin. Therefore, in some embodiments, near-infrared spectroscopy can provide information regarding tissue hemodynamics, in addition to information regarding tissue hydration and water content. Such a method also allows for the detection of subclinical edema or swelling.

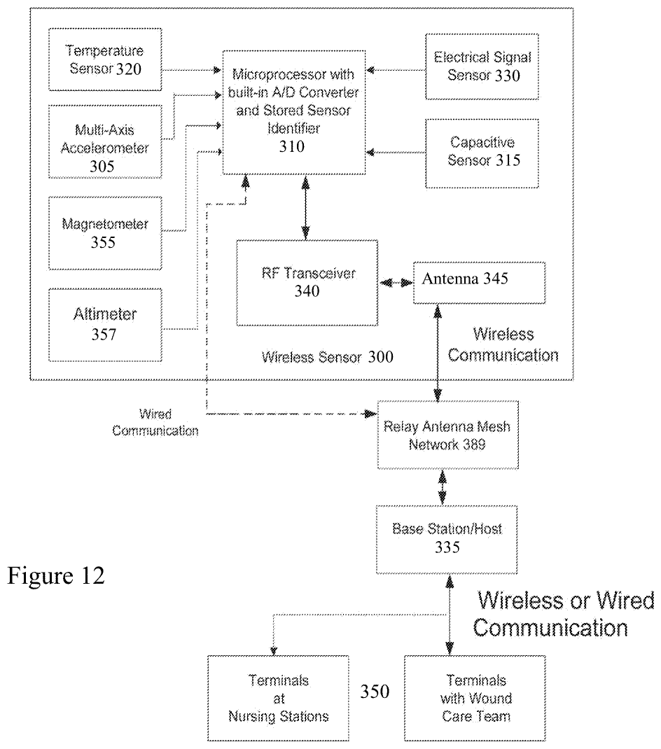

With the use of near-infrared spectroscopy, as shown in FIGS. 17A and 17B, a perfusion map of the MP can be created. One or more near-infrared light sources 1700 are used to analyze multiple physiologic processes such as TcO2, pH, and temperature. One or more infrared sensitive cameras 1705 can be used, placed sufficiently proximate to but separate from the light sources so as to receive reflected light from the MP without receiving bleed-over from the light sources. The support system then optimizes surface pressure based on the tissue perfusion map. The support system can use the data from the perfusion map to automatically optimize surface pressure distribution and alert nursing staff or caregivers of any potential abnormalities. Surface interface pressure can essentially be eliminated at areas that are identified as having compromised tissue perfusion or signs of tissue injury. In addition to helping MPs with decubitus ulcers, systems and methods disclosed herein can be useful in the treatment of MPs with burns, chronic wounds, skin grafts, flaps, and other injuries.

Laser Doppler Flowmetry can also be used for measuring perfusion in cutaneous microcirculation in some embodiments. The technique works by illuminating the tissue of interest with light from a low-power laser. The beam of laser light is scattered within the tissue of interest and some of the light is scattered back to a sensor. Most of the light is scattered by static (non-moving) tissue, but a certain percentage of the light is scattered by moving red blood cells. The light scattered by moving red blood cells is distinct from the light scattered by static tissue (i.e. it has a unique oscillation frequency), so the oscillation frequency of the backscattered light correlates with the relative number and speed of moving red blood cells. Thus, this technique can be used to measure the relative amount of moving red blood cells and measure their average velocity. This technique is completely non-invasive and can be used to interrogate subcutaneous tissue to a depth of several millimeters. If areas of abnormal perfusion are detected, the support system can automatically eliminate surface interface pressure at those locations, and caregivers can be alerted. Pressure relieving maneuvers performed by the support system can be used to promote blood flow.

Some embodiments provide an arrangement of “relay antennae” forming a mesh network, e.g., within a hospital or other medical facility, for communicating data between a wireless sensor and other device(s), e.g., monitoring terminals.

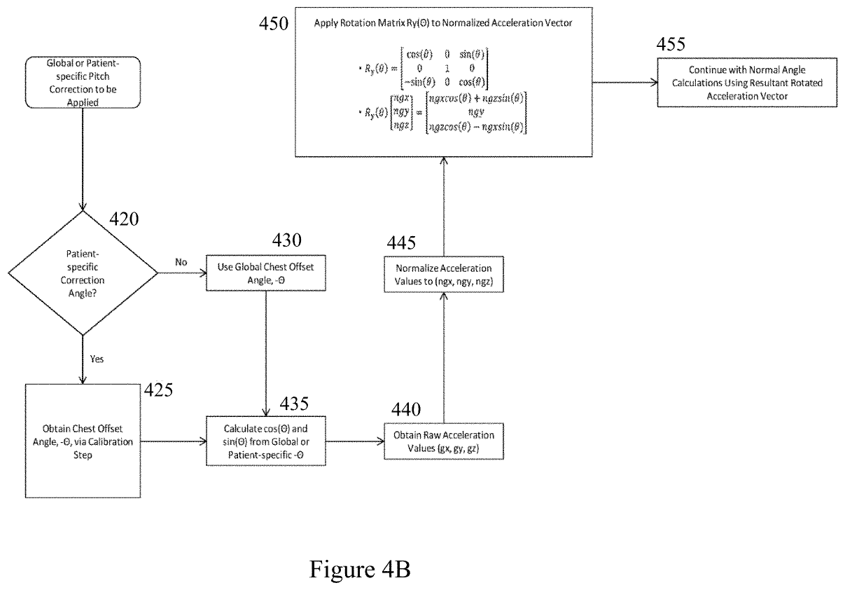

For example, FIG. 12 shows an example hardware architecture of a sensor 300 configured to transmit sensor data to receiving device(s), e.g., caretaker terminals 350, via an arrangement of relay antennae, in accordance with one embodiment. In such an embodiment, the sensor 300 comprises a multi-axis accelerometer 305, a magnetometer 355, an altimeter 357, a temperature sensor 320, an electrical signal sensor 330, and a capacitive sensor 315, all of which provide data to a microprocessor 310. The processor 310 includes an indicia which uniquely identifies the particular sensor. The processor at least collects, and in some instances processes at least some, data from the sensors and sends that unprocessed and/or processed data to an RF transceiver 340 which then transmits the data via antenna 345. Not shown in FIG. 12 is a battery for providing power for the various sensors and circuits. It will be appreciated by those skilled in the art that, for some MPs, it may be desirable to affix multiple sensors at different locations.

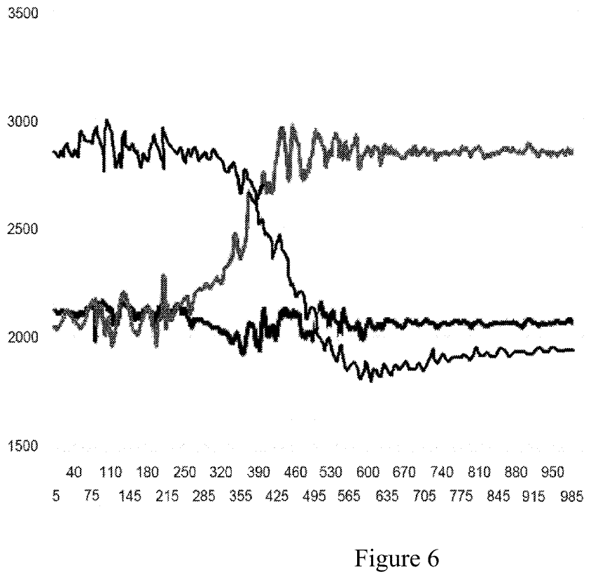

The sensor data transmitted via the antenna 345 is received on one or more relay antennae forming a mesh network 389 as described greater detail in connection with FIGS. 13A-13D. In an embodiment, the data is received on a plurality of the antennae, permitting the location of the MP-worn sensor to be determined with reasonable accuracy. The various antennae communicate their information to a base station host/server 335, which then processes the data in the manner discussed hereinafter. Depending upon the particular data, the host may then generate status updates, warnings, alarms and/or care recommendations to one or more caregivers as illustrated at 350.

With reference to FIGS. 13A-13B, an example mesh network can be better appreciated. In particular, FIG. 13A illustrates generally a plurality of MPs each wearing a sensor, while FIG. 13B illustrates the network in greater detail. The sensors communicate with one or more proximate relay antennae, and the various relay antennae forward that data to the host. As shown in FIG. 13B, network 411 is comprised of an array of relay antennas 450 that can communicate via wireless links 440 with each other to form a mesh network. MP Sensors 460 communicate via wireless links 440 with relay antennas 450 in order to send messages and receive messages from back end server 422. The backend server 422 of network 411 stores and retrieves data from a database 470 over a link 430.

Communication links 440 can be any suitable means for communicating between two relay antennas 450, between a relay antenna 450 and one or more MP sensors 460, or between a relay antenna 450 and back end server 422. Communication links 440 can be wired or wireless. The link 430 can also be any suitable communication link between the back end server 422 and database 470. The links 430 can be different in nature than the links 40 within the mesh network 11. For example, links 440 can be wireless links and link 430 may be a hard-wired link. Alternatively, the database 470 may reside within server 422, or distributed across a plurality of servers or other host computers.

Network 412 shows a more complicated mesh architecture with multiple back end servers 421 and 423. Although only two are shown, any number of backend servers can be interconnected within a single mesh network. Both of the back end servers 421 and 423 of network 412 communicate with the same database 470, although, as noted above, the database 470 can also be distributed across a plurality of machines.

In some embodiments, networks 411 and 412 are substantially isolated from one another as depicted in FIG. 13B. The isolation may be because the networks are geographically separated and the antennas in the different networks do not have sufficient power to communicate with each other. Alternatively, the networks may be co-located but isolated through software or hardware protocols that prevent the antennas of the different networks from communicating with each other. Although networks 411 and 412 are isolated, the back end servers of each network may use the same database 470. In this way, the networks may be logically treated as the same network by hardware or software systems that process data from the database.

Other devices 480 can access the database to read, write, or modify data within the database. Other devices 480 that can communicate with the database 470 can be, for example, front end components of the system that interface with hospital staff, other facility computing or data systems such as electronic medical record systems or other MP monitoring systems, or nurse call systems. The communications link 490 that other systems use to access the database may be similar to or different from data links 430 and 440. For example, communication link 490, can use an HL7 interface or other protocol commonly used for interfacing hospital data systems.

In one implementation, caregivers are able to view MP data on a display device. The MP data enables caregivers to provide more efficient and effective care, particularly as it relates to pressure ulcer prevention. From the display device, caregivers are able to view the turning status and other care parameters for one or more MPs.

Referring next to FIG. 13C, the interaction of the sensor, mesh network and host can be better appreciated, while FIG. 13D illustrates the structure of the relay antenna module. In particular, one or more sensors 300 collect MP position (including height as appropriate), orientation, ambulation and physiological data and communicates that data to a mesh network 389. The mesh network then communicates that data, either wirelessly or via wire, to the host system 335. The host system 335 typically comprises at least a processor 125 and storage 135. The host processor manipulates the received data in accordance with various algorithms as well as the stored sensor, historical and other data to determine the state of the MP. Using that information, the host processor can generate care alerts and make recommendations for MP care specific to the particular MP. The alerts and recommendations are then provided to the caregivers or automated systems, generally indicated at 350 and as described above. In at least some embodiments, the data, including both the sensor data and the analytical data, is stored for future use.

It will be appreciated that a typical use as shown in FIGS. 13A-13D involves the caregiver using the disclosed MP management software to define individualized turn, bed exit, ambulation and other protocols for one or more MPs that require monitoring. The individual protocol parameters are stored in the Database for future reference. The MP Sensor, which is disposable and wireless in some embodiments, is associated (for example, adhesively affixed) to the body of the MP being monitored. The sensor makes measurements of the MP's orientation and communicates this data, wirelessly or by other suitable means, over the data collection network previously set up in the facility. The network relays data from the MP Sensors to a server computer connected to the data collection network. Software running on the server computer collects the MP data and stores the data into a database for subsequent analysis. The MP's orientation and other MP related metrics can then be determined by analyzing the data stored in the database. The MP Management Software (also referred to as Turn Management Software) displays each MP's turn history and current status. The MP Management Software also alerts staff if any MP requires a caregiver-assisted turn. The system also has the ability to automatically document each MP's turn history (including caregiver-assisted turns and MP self-turns).

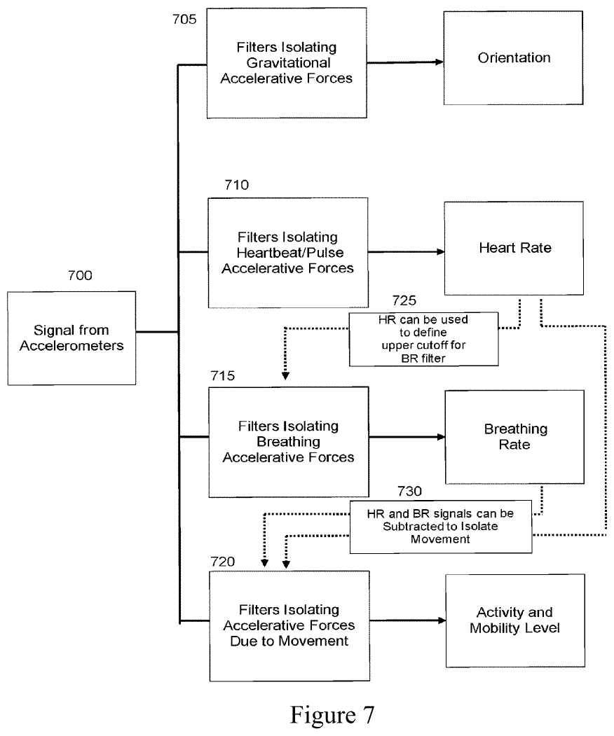

In addition to the features discussed above, an embodiment of the system of FIG. 13C can include monitoring for bed exits and falls among its functions. In such an embodiment, the sensor includes an altimeter and a magnetometer in addition to the accelerometer, and optionally other detectors. Depending upon the MP characteristic being monitored, the sensor provides data from one or more of the detectors, such as the accelerometer, magnetometer and altimeter, and provides it to the host system. The host system processes that data, for example using one or more of the algorithms described hereinafter in connection with FIGS. 14-26, by which the system uses current and historical data to determine the probability that a bed exit is likely to occur soon, or that a fall has or is about to occur. The historical data for such a determination comprises, in at least some embodiments, prior sensor data, the health status of the MP, the location of the bed and a compass heading for a reference axis of the bed, the altitude of the floor, and, optionally, recommendations and settings specific to the care of the monitored MP.

Referring next to FIG. 13D, the architecture of an embodiment of the relay antenna 491 can be better appreciated. In particular, the relay antenna 491 includes, in at least some instances, an altimeter 493 which provides elevation data to a processing unit 495. In addition, the relay antenna comprises a transceiver 497, which both receives data from the sensors proximate to it, and transmits to the host/server that data as well as the altimeter data received from the processor 510.

In addition, in at least some embodiments the relay antenna includes a wall plug 499 for providing power to the unit while also permitting extremely easy installation within a monitored facility. Further, because wall plugs in facilities such as hospitals are typically at a uniform height, the altimeters in the various relay antennae form a horizontal reference plane against which relatively small variations in the height of MP-worn sensors can be reliably detected. This assists in the detection and monitoring of bed exits, falls, and ambulation.

It will be appreciated that, while some embodiments described above include all of an accelerometer, a magnetometer and an altimeter, not all embodiments necessarily require having the entirety of these sensors.

As described previously, traditional pressure ulcer prevention protocols involve turning high-risk MPs on a regular basis, such as every two hours. In one embodiment, a turning protocol can be managed and coordinated with the aid of MP sensors. The system, methods, and devices disclosed herein provide a means for continuously monitoring the position, orientation, and activity of one or more MPs and help caregivers carry out a prescribed turning protocol. The system can display the real-time turn history of MPs and indicate to caregivers if a required turn is approaching or past due.

As discussed above, the sensor itself is a key element in some embodiments of the system, but the sensor is typically powered down prior to association with an MP and, potentially, being affixed thereto. The initialization of components, including a sensor, and their integration into any of the networks discussed above can be better understood in connection with FIG. 12.

In particular, FIG. 14 illustrates the power-on initialization and network integration of a device that is not connected to the power grid can be better appreciated. For the sake of clarity, the following discussion will assume a battery-powered device, although such devices can be powered by any suitable means capable of providing sufficient power and portability, for example photo-voltaic cells. An example of such devices are the sensors described above configured to be affixed directly to an MP. For such devices, the process starts at 1400, where the state of the device is that power is off except, for example, for a “power-on” circuit such as a phototransistor and related circuitry that responds to ambient light. At 1405, upon the removal of an adhesive liner, ambient light illuminates the phototransistor or other photodetector and power is connected to the rest of the sensor. Thereafter, at 1410, the newly-powered-on device broadcasts a request to join a network, typically with identifying information as discussed above. If a join response is received from a network at step 1415, the process advances to step 1420. If no join response is received, the process loops back to step 1410 and repeats the broadcast.

At step 1420, the device such as a sensor makes a measurement and, at step 1425, that measurement data is transmitted to a server for further processing. The transmission need not be near-real time relative to the taking of the measurement, although in many embodiments it will be. The transmission is typically, although not necessarily, made wirelessly, and is thus received at, for example, one of the antennas shown in FIG. 14 and thence to a server.

At step 1430, the device checks to see if an acknowledgement has been received, indicating that the server received the data transmitted by the device. If not, the process branches to step 1435, where a check is made concerning how many unsuccessful transmission attempts have been made. If a threshold number of attempts have not been made, the process loops back to step 1425 and the data is re-transmitted. If a threshold number of repeat transmissions has been reached, the assumption is made that the device has lost communication with the network, for example by the MP walking into a different area of the hospital, and the process loops back to step 1410 to enable the device to join whatever network is available for that location.

If the data transmission was successful, as indicated by an acknowledgement at step 1430, the device will, for at least some embodiments where power conservation is important, enter a sleep mode as shown at step 1440. The device remains in sleep mode until a predetermined event occurs and causes the device to return to full operation. Such events can be elapsed time, a fall, a signal having unusual characteristics, or what may be thought of as an “out-of-bounds” signal indicating some unusual activity by the MP.

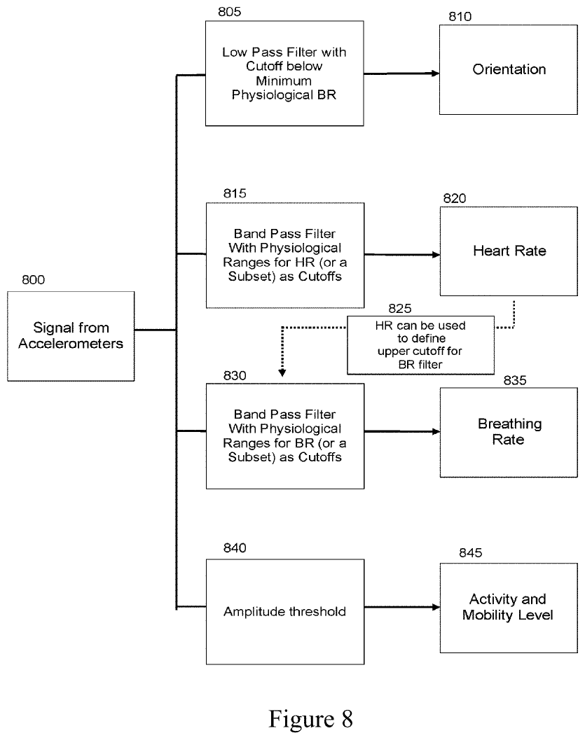

Referring next to FIG. 15, the power-on initialization of a network device that is not plugged into the power grid. Such a device may be, for example, the antenna/transceiver combinations that comprise part of the mesh network of FIG. 15A-15B. The device starts with power off at 1500. Reasonably promptly upon application of power at 1505, the device initializes and broadcasts a request to join a network, shown at 1510. While in some embodiments the device will be pre-configured for the type of network, in at least some embodiments the device does not know in advance what network is available, and looks for a “join” response from any network. The device typically provides, as part of the broadcast, information that identifies it for at least network purposes. Upon receipt of a join response at 1515, the device advances to the device main loop, referenced as 1400 and described in connection with FIG. 14. If no join response is received at 1515, the process loops back to 1510 and the broadcast step is repeated until a join response is received, or the process is otherwise terminated.

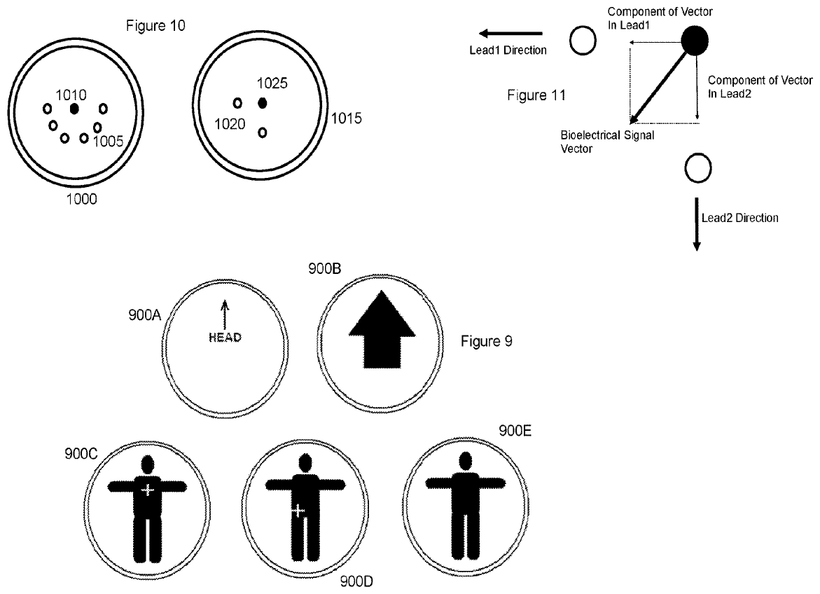

Referring next to FIG. 16, the device main loop referred to above in connection with FIG. 6 can be better appreciated. The process starts at step 1600 and advances to step 1605, where a query is generated asking if there is an unknown route to a destination. In this context, “route” refers to network topology, where each retransmission represents a “hop” and the most direct route is indicated by the lowest number of hops. Typically, the destination is the location of a sensor or other device capable of providing relevant data to the system, and may be affixed to an MP moving about a hospital or other monitored area. If there is a route that is not known, such that there is a new route to a destination, the process advances to 1610 and the device broadcasts a request to neighbors of the destination for information about the logical distance to that destination. The process then loops back to 1600 so that the neighbors can supply the necessary information concerning logical distance.

If all routes are known, such that there is no new route or the new route data has now been entered as a consequence of the loop back to step 1600, the process advances to step 1615, and the device receives information regarding distance to a destination. If the device receives such distance information, the device determines distance on its own at step 1620 by adding a value representative of one additional hop to the minimum distance reported by the neighbors, and then loops back to step 1600.

At step 1625, if the device receives a request from a nearby antenna for distance information to a destination, the process branches to step 1630 and the device broadcasts its depth to a destination, if known, after which the process loops back to step 1600. At some point for at least some embodiments, a status report is due to be sent as shown at step 1635. If so, at step 1640 the device transmits the status report to the network, for example via a nearby neighbor, and the status report is then communicated to the appropriate server as shown at step 1640.

In some embodiments, messages will be received by the device from, for example, an MP sensor or a server, or other device such as a query from a hospital system. If the message is for the device, the message is acted upon. However, if the message is not for the device, the device relays the message to neighbors en route to the appropriate destination, as shown at step 1645.

System Architecture and System Use

The following description elaborates on the aspects discussed above, and further describes the architecture of a monitoring and reporting system for managing and coordinating MP-turning protocols in accordance with one embodiment.

Some embodiments provide a system for monitoring MP orientation and for alerting caregivers for the need to turn an MP being monitored which is easy to install, easy to maintain, easy to interact with, and reliable. As shown in FIGS. 1-4, an example embodiment of a system can comprise the following components:
MP Sensors
Data Collection Network
Back-end Server
Database
MP Management Software (Turn Management Software)

In a typical use of such a system, the caregiver uses the turn management software to define an individualized turn protocol for one or more MPs that require monitoring. The turning protocol parameters for these individualized protocols are stored in the Database for future reference. The MP Sensor 300, which is disposable and wireless in some embodiments, is associated (for example, adhesively affixed) to the body of the MP being monitored. The sensor makes measurements of the MP's orientation and communicates this data, wirelessly or by other suitable means, over the data collection network previously set up in the facility. The network relays data from the MP Sensors to a server computer connected to the data collection network. Software running on the server computer collects the MP data and stores the data into a database for subsequent analysis. The MP's orientation and other MP related metrics can then be determined by analyzing the data stored in the database. The Turn Management Software displays each MP's turn history and current status. The Turn Management Software also alerts staff if any MP requires a caregiver-assisted turn. The system also has the ability to automatically document each MP's turn history (including caregiver-assisted turns and MP self-turns).

A more complete description of each of the system's components is given in following sections:

MP Sensor

In an embodiment, the MP Sensor is a single-use, disposable, wireless device that can be adhesively affixed to an MP's skin. The sensor may be reversibly associated with the MP in any fashion. In an embodiment, the sensor itself is comprised of several components: a 3-axis accelerometer to measure MP orientation and activity; a phototransistor that measures ambient light levels and turns on the device when the packaging and/or adhesive liner is removed; a capacitive contact sensor that enables the device to sense when it is attached to skin and sense when it is removed from skin; LED indicators to visually communicate information to the caregiver; a microcontroller for automated data collection, analysis, and storage; an RF radio for transmitting and receiving messages; and a common CR2032 coin-cell battery for providing electrical power. In an embodiment, the skin-contacting portion of the MP sensor is a commonly used polyurethane dressing having an acrylic adhesive. However, any other suitable adhesive may be used.

In an embodiment, the MP sensor is enclosed in an optically opaque pouch that is easily opened by the caregiver at the time of use. While in the packaging, the majority of the circuitry of the sensor need not be powered in at least some embodiments. In this way the shelf life of the device's non-chargeable or non-rechargeable battery can be as much as several years. After removing the device from the packaging, the caregiver needs only to remove the adhesive backing from the underside of the device, exposing the adhesive surface, and then apply the device to the upper torso of the MP. Indicia on the MP Sensor indicate the proper orientation of the device with respect to the MP. The device contains a photosensitive electronic circuit that detects ambient light. When the device is removed from opaque packaging the ambient light incident on the photosensor activates the device and power is then supplied to the main electronic circuits of the device. Alternately, the adhesive backing can be opaque and when the opaque adhesive backing is removed it enables ambient light to strike the photosensitive circuit. The photosensitive circuit turns on the device when ambient light is detected.

In an embodiment the device provides a visual indication to users that it has successfully powered-up. One method is to provide one or more LEDs that briefly illuminate when the device has powered up.

In an embodiment the device automatically joins the wireless Mesh Network once power has been provided to the device, as discussed in connection with FIG. 15. In an embodiment the MP Sensor communicates with a nearby Relay Antenna having the strongest RF signal, although other protocols for managing communication between the sensor and the remainder of the network are acceptable as long as communication is reasonably maintained.

In an embodiment the device indicates to users that the sensor has successfully joined the Mesh Network. A method in an embodiment is by showing a pattern of scrolling LEDs (repeatedly turning each LED on and then off in sequence).

In an embodiment the device conserves battery power. After successfully joining the RF network, the MP sensor spends most of its time in a very low power “sleep” mode. On a regular schedule, for example, every ten seconds, the sensor may briefly “awaken” to make measurements and transmit the measured data to a nearby Relay Antenna. Once affixed to an MP, the sensor continues to make measurements every ten seconds until the battery is depleted over a period of greater than about three weeks.

Since RF communication takes significant power, in some embodiments the sensor can measure and store several MP data readings with the RF radio circuitry off. Only after several measurements have been taken would the device turn on the radio and transmit from memory some or all of the measurements recently stored.

Alternatively, sections of the circuitry could continue to make measurements while most of the circuitry is in a low power “sleep” mode. The device could “awaken” due to events detected by the portion of the circuit that was not put to sleep. Such events could be if the magnitude of acceleration is above or below some threshold, the direction of acceleration changes by some amount, the ambient light measured by the device rises above or below some threshold, the device detects that physical contact with the MP is lost, or a combination of more than one these or other events.

In addition to the phototransistor, two additional sensors reside within the MP Sensor in at least some embodiments: a three-axis accelerometer and a capacitive contact sensor. The three-axis accelerometer is a semiconductor device that is sensitive to accelerative forces, including gravitational forces, applied to the device. By sensing the direction in which gravity pulls on the device, the orientation of the device and thus the position/orientation of the MP can be determined. In order to accurately determine the position/orientation of the MP, it is necessary for the three-axis accelerometer to be oriented correctly with respect to the MP either at the time of affixing the sensor to the MP, or following measurements by the sensor and processed by the system. To aid in orienting the sensor with respect to the MP, in some embodiments the sensor can have indicia to allow for proper placement on the MP. In particular, the housing unit, which houses the accelerometer, can provide a surface for the desired indicia which can be as a mark, arrow, or icon indicating proper orientation. In such embodiments, the orientation of the accelerometer with respect to the housing unit is known or can be determined.

A capacitance sensor can be located just inside the bottom surface of the device in some embodiments, and is used to sense the change in capacitance that occurs when the device is affixed to an MP's skin. The capacitance data may be used to determine if the device is attached to (or has become unattached from) the MP. If the device is not attached to the MP, then orientation measurements made by the sensor cannot be trusted to correspond to the present orientation of the MP. The orientation and contact status of the sensor can be displayed in the User Interface of the Turn Management Software used by the caregivers.

If the sensor is wireless, the MP is unencumbered by an umbilical extending from the device. The MP is free to move about, in-bed, or out-of-bed. The MP's movement, position, orientation, and activity can be continuously and seamlessly tracked, regardless of the support surface they are on.

In an embodiment the sensor is sealed so that an MP wearing the device may shower or bathe as usual. Both optical sensors and capacitance sensors can operate through a sealed enclosure.

In an embodiment the skin-contacting portion of the device is a polyurethane dressing having an acrylic adhesive. This type of dressing is very commonly used in medical practice. The MP may wear the device continuously for many days. Other types of adhesive systems may be used, including hydrogels and silicone adhesive systems.

The MP Sensor can communicate wirelessly with any neighboring antenna. In an embodiment the MP sensor can transmit and receive messages greater than about five feet. However, many structures in the environment (walls, cabinets, carts, and even the MP's own body) may reflect and/or attenuate the RF transmissions. Practically, it can be desirable to have antennas positioned about every 25 feet to ensure that at least one Relay Antenna is within range of any MP sensors. If the MP is ambulating, the MP Sensor may not be able to communicate with the relay antenna with which it was initially communicating. If this occurs, in an embodiment the sensor automatically begins communicating with a different, near-by relay antenna that has the strongest RF signal. In this way, the sensor will always stay connected to the wireless mesh network of antennas, selecting a new antenna with which to communicate, as necessary. The messages and the MP data they contain will automatically find their way to the server computer and be stored in the SQL database, no matter what Relay Antenna is communicating directly with the MP Sensor.

It is preferable that each MP Sensor has a unique serial number that is assigned to the device either during the manufacturing process or at any other suitable time. The sensor can be uniquely identified on the RF network by this serial number. Alternatively, when the sensor is turned on and joins the wireless mesh network, it may be assigned a unique network address by the back-end server (or other device on the network), by which it is subsequently addressed. Alternatively, the MP sensor can randomly select a unique identifying number after it powers-up. The MP sensor and back-end server can thereafter confirm that the randomly chosen identifying number has not already been used. If the randomly chosen number has already been used by another device in such an embodiment, then the process of choosing a random number is repeated until a unique, previously unused number is determined. The unique serial number can be provided to users, such that the unique device serial number can be linked to an MP, bed, or other MP identifier. In some implementations, the link between the unique device serial number and the MP can be made automatically. In a preferable approach for automatically linking this information, the physical location of the sensor is automatically determined (i.e. the system can determine what bed the sensor is being used on through signal strength analysis, triangulation, and other means). The system then pulls bed/MP information for an electronic health record or ADT (Admission, Discharge, Transfer) database to determine what MP is associated with the bed.

In some embodiments, the MP sensor transmits no MP-identifying information (for example, name, electronic record number, etc.) over the wireless mesh network, since then it is not necessary to encrypt transmissions in order to protect MP privacy. Alternatively, all transmissions or a subset of transmissions can be encrypted just prior to transmission by a means familiar to those skilled in the art.

Data Collection Network

Data measured by the MP Sensor is typically communicated to a back-end server (a computer or other device responsible for storing the data in a database) as discussed. The data can be communicated from the sensor to the back-end server over a wired network or over a wireless network. A wireless data collection network is preferable in some embodiments because wires or cables do not need to be routed within the facility. Further, as mentioned previously, it is preferable in at least some embodiments that the MP sensor itself be wireless. A properly designed wireless network could be conveniently installed in a short amount of time. Unfortunately, common wireless networks are often unreliable. Common wireless networks may experience interference from other RF communication equipment, other environmental RF noise sources, and environmental obstructions like walls, cabinets, furniture, equipment, and humans moving through the facility. In an embodiment a wireless network would have features that would make it very reliable in spite of these modes of interference.

In an embodiment, a reliable wireless network is created by having a high level of redundancy in the network. An array of antennas arranged in a one, two, or three dimensions, in which any antenna can communicate with one or more nearby antennas form a mesh-like (or web-like) architecture, such as shown in FIGS. 13A-13B. In some arrangements, the antennas do not need to be positioned precisely on a regular grid, but instead, can be plugged into available power sources approximately every five feet to 200 feet. The relay antennas can be powered by any suitably reliable source, such as facility line voltage, a single use or rechargeable battery, a photovoltaic system, etc. Redundant power sources offer a good solution in at least some embodiments and comprise, for example, the facility's line voltage, plus a single use or rechargeable battery inside the device for back-up in the case that the facility power fails.

In an embodiment, the messages sent to or from MP Sensors over the mesh network are relayed from the source to the destination through sequential transmissions (hops) from one Relay Antenna to the next. Many possible routes from source to destination may exist within the mesh network of antennas. For simplicity, “antenna” or “Relay Antenna” as used herein will be understood to include the associated transceiver electronics unless indicated otherwise by context. Even in a one-dimensional mesh-network, redundancy can be achieved by allowing transmissions to hop over one or more antennas physically located between a transmitting and receiving antenna. In a mesh network architecture, even if one or more antennas are not working, there may still be one or more alternate routes through the mesh network that can be used to transfer the message from the source to the destination.

The web-like architecture of the mesh network ensures there are redundant pathways between the sensor and server. If a Relay Antenna is broken, is removed, experiences RF interference, or is obscured by equipment, cabinets, walls, or people, then messages may be automatically routed around the non-functioning antenna via one of the other redundant routes. The route used to relay a message between the source and destination is ideally determined cooperatively by the individual antennas that make up the mesh network, so as to select a route having a combination of good RF signal strength and few hops. Alternatively, a router device can communicate possible routes to the antennas that comprise the network. The router device could be one or more of the relay antennas or even software running on the server computer that has been given additional responsibility to determine the possible routes and communicate these routes to the relay antennas.

Possible routes can be determined cooperatively without the need for a router device. As discussed in connection with FIG. 16, each antenna can determine its logical distance from the destination of a message. The logical distance of an antenna to a destination is the number of times a message must hop in order to be relayed from the antenna to the destination. The logical distance of an antenna that can communicate directly with the destination is one. The logical distance of an antenna to a destination with which communication cannot be made directly may be determined by an antenna by adding one to the minimum of the logical distance(s) of neighboring antenna(s) to that same destination. An antenna may determine the logical distance of neighboring antennas by transmitting a request to nearby antennas for their logical distance to a specific destination. When an antenna receives a message for a given destination, then, if possible, the antenna and associated transceiver merely transmits the message directly to the destination. Otherwise the antenna/transceiver transmits the message to the neighboring antenna/transceiver having the minimum logical distance from the destination. Possible routes from an antenna to a destination can be limited to those routes that have sufficient RF signal strength to ensure reliable communication between each of the relay antennas along the route. Additionally, routes may be ranked in order of preference by an algorithm that weighs the number of hops and also weighs the link quality of each hop along the route.

In an embodiment, no intervention by the caregiver or IT staff of the facility is required when individual Relay Antennas are temporarily non-functional. When the antenna again becomes functional, it will automatically rejoin the mesh network and preferred routes between the server and MP sensors will automatically be determined. In this way, the mesh network is “self-assembled” and “self-healing”. If an antenna is trying to communicate with another antenna that is no longer responding, the first antenna may re-determine its logical distance from the destination and send the message to an alternate neighboring antenna.

Even in a network that is highly redundant, means for ensuring data integrity during communications over one hop from an antenna to a neighboring antenna is desirable. It is preferred that antennas determine if a message has been corrupted during transmission. A preferred method for determining if a message transmitted from one relay antenna to another has been corrupted it to compute a checksum of the message to be sent and include the checksum with the transmitted message. A numerical value may be assigned to each character that comprises a message. For example, the American Standard Code for Information Interchange (ASCII) assigns a unique value for each alphanumeric symbol commonly used in communication. The checksum may be a simple sum of the numerical values assigned to each of the characters of the message, a modulo sum of the numerical values assigned to each of characters of the message, or may be a more elaborate cyclical redundancy check (CRC) that is one or more bytes in length. Those skilled in the art of data communication are familiar with various check summing algorithms. The receiving antenna may re-calculate the checksum of the message and compare the re-calculated checksum with the checksum transmitted with the message. If the checksums agree, the receiving antenna can notify the transmitting antenna of the successful receipt of the message by transmitting an acknowledgement to the antenna that sent the message. If the antenna that sent the message does not receive an acknowledgement in some period of time, the antenna that sent the message may try to re-send the message. Further, it is preferable for the antenna that sent the message, after one or more un-acknowledged attempts to send the message, to try to send the message via an alternate route to the destination.

In a mesh network architecture in which there are multiple transmitters and multiple receivers that may be attempting to communicate multiple messages there may be times when collisions occur. Collisions occur when two transmitters attempt to communicate at least a portion of their messages simultaneously. In an embodiment, the sensors and antennas use techniques to reduce the likelihood of collisions. One method for avoiding collisions is to perform a clear channel assessment (CCA), and can be implemented in some embodiments. During a CCA the device checks for RF transmissions already in progress or alternately merely checks for significant RF power before initiating a new transmission. If it is determined that a clear channel is not present, the device waits for a period of time before trying again. Prior to each attempt, the device can again perform a CCA. The time delay between subsequent attempts may be a fraction of the time it takes to send a message up to several times the time it takes to send a message. For messages of about 30 characters in length transmitted at a data rate of 250 kilobits per second, this delay time may be from about 10 μs to 10 ms. The time delay between subsequent attempts may be fixed or may increase in some fashion, such as linearly increase or exponentially increase. The time delay between subsequent attempts may be random or pseudorandom in duration. If the transmission of a message is not acknowledged, it may be because a collision occurred even though a CCA was made prior to transmission. If a message is re-transmitted because it was not acknowledged, similar time delay variations as used after failed CCA may be used between subsequent transmission attempts.

A single mesh network can comprise just a few antennas on up to hundreds of relay antennas. Further, multiple mesh networks can be configured within a single facility. Each mesh network may communicate messages from MP sensors to a different back-end server. The back-end servers may store the data contained within the messages to different databases or alternately may communicate the data to the same data base. By communicating data from physically isolated mesh networks having different back-end servers into the same database, the physically isolated mesh networks can be logically treated as a single network by the system. In this way, wards, hospitals, and even hospital chains can be monitored as if they were a single network, if desired.

In an embodiment the system allows facility staff to administer the system. MP Sensors and Relay Antennas can generate regular status reports and send them as messages to the back-end server. Status reports can be generated when specific events occur, such as when a device (sensor or antenna) is powered on, when a device joins or rejoins the network, or when new neighboring devices are detected. Status reports may also be generated at regular time intervals and include information like how many messages were received, how many messages where corrupt, how many messages needed multiple attempts to transmit, or how times an antenna needed to recalculate its routes. In an embodiment, software running on the back-end server collects and analyzes the reports made by sensor modules and/or antennas. In an embodiment the back-end server communicates alerts to facility staff based on the analysis of the reports from sensor modules and/or relay antennas. The back-end server alerts the facility staff if some period of time has passed since the last status report from a relay antenna or MP sensor. Such an alert would occur if an antenna was removed, broken, or experiencing some type of interference. The back-end server could provide other alerts based on the information provide in the status reports. The back-end server could assimilate the data from the reports and provide regular summary reports of the status for the system. The back-end server could communicate the alerts and summary reports via text messages, email, a paging system, or website. Alternately, it provides the alerts and reports as files available to users that have access to the back-end server.

It is desirable that, in at least some embodiments, the RF network use low power transmissions so that battery life of the MP sensor and relay antenna be extended. Low power transmissions also limit RF interference induced in other equipment used in the facility. Low power transmission also limits the interference with medical and electronic devices used by the MP; for example, pace makers, defibrillators, cell phones, or personal computing devices. A preferable frequency and power level is about 2.4 GHz and about 1 mW.

Back-End Server Software and Database

In an embodiment messages sent by the MP Sensors are communicated by the data collection network to a back-end server computer running data collection software. Preferable, the back-end server has a means for connecting to the data collection network. For a wired data collection network, a Network interface card may connect the back-end server to the data collection network. For a wireless data collection network, an RF transceiver may connect the back-end server to the data collection network. The primary purpose of the back-end server is to collect MP orientation data and save the data into a database. Other tasks may be performed by the back-end server such as assigning network addresses to MP Sensors and Relay Antennas as they join the mesh network, and collecting other miscellaneous data from the MP Sensors and Relay Antennas such as status reports and other network information.

In an embodiment, the back-end server may send messages to devices comprising the data collection network. Such messages may be commands to perform a self-test, turn off the main circuits of the device, or to modify operating parameters such as radio power, measurement frequency, or other operating parameters.

A system in which more than one back-end server is provided for each physically isolated mesh network is preferable for some embodiments in that it increases reliability through redundancy. In normal operation, messages to and from an MP sensor may be routed through the back-end server that has the shortest logical distance from the MP sensor. This architecture also has the benefit of increasing total bandwidth since message traffic is divided between the multiple back-end servers. In the case that one of the back-end servers stops working, or its connection to the database is lost, the message traffic will may automatically be re-routed through other available back-end servers.

It is also desirable that the database be reliable. If the database is stored on hard disk drives, then the use of mirrored hard drives or other configurations of redundant disk arrays is preferable. In some embodiments, the hard drives or hard drive arrays can have redundant drive controllers and redundant interfaces to the one or more back-end servers, or otherwise provide high availability.

It is desirable that any of the one or more back-end servers have software or other means that monitors the performance of the back-end server. The back-end server monitor can automatically reset or restart the back-end server if the back-end server does not function properly. In an embodiment the back-end server monitor is capable of alerting facility staff via email, text messages, paging systems, or a website if problems are detected.

In an embodiment, the data exchanged between back-end server and the database contains no MP-identifying data such as MP name or MP electronic medical record number which obviates any concern about data security on any data links between the back-end server and the database.

With the operation of the sensor and network in mind from the foregoing, the following discussion of a sensor-assisted turn management system can be better appreciated. It will be appreciated by those skilled in the art that many aspects of the disclosed turn management system are implemented in software operating to define the functions performed by the servers, database(s), and related hardware previously discussed. When implementing a sensor-assisted turn management system, there are various turning parameters, algorithms, and data analysis techniques that can be incorporated to substantially improve the clinical usability and utility of the turn management system. These items are discussed hereinafter.

In general, turning protocols are based on regularly alternating an MP's surface pressure distribution between two or more body regions, such as their back side, right side, or left side. One embodiment enables the pressure distribution across body regions to be monitored and managed in a more sophisticated, accurate, and reliable way.

Turning protocols are generally based on a desired turn period, which is set by caregivers. A turn period reflects the amount of time that an MP can stay on any given body region before a turn is required under the protocol set by the caregiver. As shown at 1700A-B in FIG. 17, in at least some embodiments, turn periods are a configurable setting. Turn periods can dynamically change based on MP, environment, or institution specific variables. For example, the turn period may change based on the MP's risk of developing a pressure ulcer (which can be a value manually entered into the system, automatically extracted from a medical record, or automatically calculated based on sensor data collected in 1700A). Still further, the turn period can dynamically change based on the MP support surface, MP's health status, presence of existing pressure ulcers, time of day, caregiver staffing ratios, or virtually any other care-related variable.

Still referring to FIG. 17, for each MP, or group of MPs, caregivers can define acceptable body regions as shown at 1705. In one embodiment, angle thresholds can be used to define the body regions. The orientation of the MP (across one or more axes) is determined by one or more sensors that are associated with or affixed to the MP, for example, the sensors that are described in Appendices A and B, the MP sensor(s) communicate MP orientation data to a host, which compares the calculated MP orientation to pre-defined body region threshold angles, so as to determine the body region(s) that are currently pressurized. For example, if it has been defined that the back side angle thresholds are −30 and +30 degrees, the MP will be considered to be lying on their back when they are between those angles.

It should be noted that defining body region threshold angles is not a requirement. It is possible to simply display the angle of an MP (in one or more axes), without defining the MP's position. However, providing a gross assessment of an MP's position may make the system easier to use and may make the system more consistent with traditional turning protocols (which typically define a back side, left side, and right side).

In one embodiment, the cumulative amount of time spent in any given body region can be calculated by the monitoring system. As shown at 1710, in an embodiment the system assigns a timer to each defined body region. The system then can calculate the amount of uninterrupted or continuous time a given body region spends in either a compressed or decompressed state, as well as the total body region time accumulated, for either compression or decompression, within a specified time period (i.e. total amount of time spent in a given body region over 24 hours). Time thresholds for providing alerts and notifications to staff can be programmed into the monitoring system.

Typically, a turn is defined as moving from one body region to another, such as moving from the back side to the right side. When angle thresholds are used to define body regions, a potential problem arises when low magnitude turns (i.e. “microturns”) occur around the defined body region threshold angles. For example, if it has been defined that the back side angle thresholds are −30 and +30 degrees, an MP may get credit for turning if they move from +29 (back side) to +31 degrees (right side). Although the MP may have technically moved between body regions, they moved by only 2 degrees, which probably is not a clinically significant turn. Some embodiments provide methods for determining when an adequate turn has been performed, and thus addressing the problem of “microturns.”

In one embodiment, caregivers can define the minimum MP orientation change that is required in order to conclude that an adequate turn has been performed. Returning to our previous example, if a minimum orientation change had been defined (i.e. >15 degrees required for a turn), the MP would not have received turn credit for moving from +29 to +31.

In implementations where it is desired that minimum orientation changes are required for turns, the system can be designed such that the body region threshold angles also dynamically change. Returning again to the prior example, if an MP is at +29 (back side) and a minimum orientation change of 10 degrees has been incorporated into the system, the use of a dynamic body region threshold angle would require that the MP rotate to +39 degrees before they are considered turned onto their right side.

In clinical practice, MPs generally don't stay fixed at a specific angle within a body region for an entire turn period. In reality, an MP's orientation typically fluctuates within a body region during any given turn period. In some instances, an MP may also very briefly rotate onto a different body region, before quickly returning back to the original body region. These quick “turns” may, or may not be, clinically significant. In some implementations, it may be desirable to compute the average orientation angle that an MP maintains during a given turn period. This orientation averaging may be particularly relevant if dynamic body region threshold angles and minimum orientation angle changes are being used. For example, if an MP is lying on their back (i.e. initially defined as −30 to +30 degrees), and their average orientation over the turn period is +25 degrees, the MP would need to turn to at least +35 degrees in order to get credit for a turn and to be considered on their right side (assuming a 10 degree minimum orientation change).

In some embodiments, it may be desirable to set a minimum amount of time that an MP must spend within a new body region before getting “credit” for a turn. For example, if an MP changes position from +25 degrees (back side) to +35 degrees (right side), but only maintains that new position for a few moments before returning back to +25 degrees (back side), this quick turn may not be a clinically significant in terms of, for example, permitting decompression of the body region in order to ensure that turns are clinically significant, a threshold amount of time may need to elapse before credit is given for a turn. For example, it may be defined that a minimum turn threshold time of 10 minutes is required before a turn is counted. After any given turn, if an MP turns back onto the prior body region before the minimum turn threshold time has elapsed (i.e. 10 minutes), then credit for the turn is not given. Alternatively, a sequence of turns may be accumulated to aggregate either compression time, decompression time, or both, to permit a more complete assessment.

The concept of incorporating minimum turn time thresholds relates to tissue decompression times. The decompression time reflects the amount of time that a given body region needs to be offloaded before it is considered fully decompressed/re-perfused. Full decompression implies that adequate tissue reperfusion of a given body region has occurred, and thus the risk for ischemic injury has been negated. In some implementations, a decompression time can be set and may be configurable. For example, a user such as a caregiver may choose a decompression time of 15 minutes, which implies that if a body region is decompressed for at least 15 minutes, this body region will be considered fully decompressed/reperfused. After the decompression threshold has been surpassed, the MP may be allowed to turn back onto the decompressed body region.

In some implementations, each body region may have an individual timer that reflects the degree of compression/decompression permissible for that body region, as shown at 1710 in FIG. 17. The rate at which the body region timer counts up or down may vary depending on the clinical scenario. In addition, the rate at which a body region timer counts up may be different than the rate at which it counts down. For example, consider a situation where the turn period has been set to 120 minutes, with a decompression time of 15 minutes. If an MP moves onto a specific body region, the body region timer may begin counting up at a rate of 1 minute for every minute of real-time, as shown at 1715 in FIG. 17. At 120 minutes, an alert may be provided to caregivers indicating that a turn is due. In addition, various alerts (potentially escalating) may be provided to caregivers as the timer approaches 120 minutes and/or after the timer exceeds 120 minutes. Beyond 120 minutes, the timer may continue to count up at the same rate, a different rate, or may stop counting up altogether. In some implementations, once the turn period has been exceeded, the body region timer may indicate only the past due time or alternatively can indicate the total time elapsed on a given body region.

In some implementations, if an MP were to turn off of a body region, the body region timer from the previously compressed side may begin counting down, while the body region timer from the newly compressed side may begin counting up, shown at 1720 and 1725 of FIG. 17. The rate at which the timer counts up/down can be configured, and may be related to the desired tissue decompression time. For example, consider an MP on a 120-minute turn period with a desired decompression time of 15 minutes. If this MP accumulated 120 minutes of time on a body region before turning, the previously compressed body region timer would count down at a rate of 8 minutes for every 1 minute of real-time (i.e. the body region timer would be reach 0 minutes after 15 minutes of real-time). If the MP were to turn back onto a previously compressed side before the body region timer had reached 0 minutes, the timer would again start counting up at a rate of 1 minute per minute of real-time (or any other programmed rate).

In some implementations, the body region timers may have maximum and minimum values. The maximum value may be related to the turn period. For example, if the MP is on a 120-minute turn period, the body region timer may max out at 120 minutes. Therefore, if an MP is on a given body region for more than 120 minutes, once the MP turns, the body region timer will begin counting down from 120 minutes at a given rate (i.e. 8:1 for a 15 minute decompression time). In other implementations, the body region timer has no maximum values, and the countdown that occurs upon turning will begin at whatever the total elapsed time is for the body region timer.

To further illustrate how body region timers work, consider an MP on a 120-minute turn period with a 15-minute decompression time, where the given MP compresses a body region for 30 minutes. If this MP then turns onto a new body region, the previously compressed body region timer will count down from 30 minutes at a rate of 8:1, such that the body region will be considered full decompressed within 15 minutes. If the MP turns back onto the previously compressed side before complete tissue decompression has occurred, the body region timer will begin counting up from the new starting time (i.e. if the turn was only maintained for 2 minutes, the body region timer will begin counting up from 14 minutes, given that the MP was awarded 8.times.2 minutes=16 minutes of decompression).

In some implementations, the body region timer might not begin counting down until a threshold amount of offloading time has occurred. For example, if an MP performs a turn, the body region timer from the previously compressed side might not start counting down until the turn has been maintained for a threshold amount of time (i.e. 5 minutes). After the threshold has been met, the body region timer may begin counting down at any rate.

It should be noted that the rate at which a body region timer counts up or down does not necessarily need to be linear. The body region timer can progress in a stepwise, exponential, or logarithmic fashion. The rate at which the timer counts up/down can vary depending on virtually any care-related parameter, such as MP risk level, presence of existing pressure ulcers, health status of MP, support surface type, time-of-day, caregiver staffing ratios, location in the hospital, etc., as shown at 830. The body region timers can also function differently for different body regions, such that each body region timer may operate with a different set of rules. Furthermore, MP, caregiver, or environmental factors can influence each body region timer. The body region timers may also be influenced by the frequency or magnitude of turns. For example, if an MP turns from +31 degrees (right side) to +29 degrees (back side), the body region timer for the previously compressed side may count down at a relatively slow rate, such as 2:1. However, if the MP were to turn from +31 degrees (right side) to −31 degrees (left side), the body region timer for the previously compressed side may count down at a relatively fast rate, such as 16:1. In this scenario, the higher magnitude turn is awarded a faster decompression time because the higher magnitude turn is associated with better pressure relief. There are various alternative implementations of body region timers, which are all possible given the teachings herein.

In one embodiment, caregivers are provided with various notifications and alerts (potentially escalating) when an MP turn is approaching or past due. Described herein is a method for pausing alerts and notifications. When an alert/notification is issued, a user may choose to pause or “snooze” the alert/notification for a preset amount of time. This pausing functionality can be useful in situations where a turn is not immediately possible due to MP or caregiver circumstances.

The systems, methods, and devices described herein can be used to help manage, coordinate, and optimize turning protocols. In some implementations, it may be desirable to analyze the historical turning behaviors for one or more MPs, and to determine how closely a given turning protocol is followed. Described herein are novel ways of determining how closely a turning protocol is being followed. These data analysis techniques can be applied to a single MP or a group of MPs. There are various ways modifying the formulas outlined below, but the general concepts remain the same.

One method of measuring how closely a turning protocol is being followed is to calculate the compliance, which can be done in several ways. Below are four alternatives for calculating compliance:

1. Compliance (% Time):

Compliance can be calculated as a function of the percent of time that an MP is compliant with a turning protocol. In the equation below, the “total amount of time that an MP is compliant” is calculated based on the total amount of time accrued when the MP is in a state where the turn period has not expired. The denominator reflects the total time amount of time that an MP was monitored (i.e. total time accrued in expired and non-expired turn period states). For example, if an MP was monitored for a total of 24 hours and 16 of those hours occurred in a state where the turn period had not expired, the compliance rate would be 16/24=66%

Compliance (% time)=Total amount of time that at MP is compliant Total amount of time that an MP is monitored

2. Compliance (% Time)+Grace Period:

Compliance can be calculated as a function of the percent of time that an MP is compliant with a turning protocol, with the incorporation of a grace period. A grace period can optionally be incorporated into the compliance calculation, such that the calculated compliance is not negatively impacted until the grace period is surpassed. A grace period provides a small amount of time for nurses to perform a turn after a turn period has expired, whereby if the turn is completed before the grace period expires, the compliance is not negatively affected. In the equation below, the “total amount of time that an MP is compliant” is calculated based on the total amount of time accrued before a grace period is exceeded. The denominator reflects the total time amount of time that an MP was monitored (i.e. total time accrued in expired and non-expired turn period states). For example, if an MP was monitored for a total of 24 hours and 20 of those hours occurred before a grace period expired, the compliance rate would be 20/24=83%. Once the grace period expires, the MP may be considered non-compliant for the total time elapsed since the turn period first expired. For example, if the grace period is set at 15 minutes, and an MP is turned 16 minutes after the turn period expired, the MP may be considered non-compliant for the full 16 minutes. However, if the previous MP is turned 14 minutes after the turn period expired, they will not accumulate any non-compliance time. However, in other implementations, if the grace period expires, the MP may be considered non-compliant for time accrued only after the grace period expired, as opposed to the total time since the turn period expired. In general, the incorporation of the grace period will increase the calculated compliance rate.

Compliance (% Time)+GP=Total Time that MP is Compliant Total Time that an MP is Monitored

3. Compliance (% Turn Periods):

Compliance can be calculated as a function of the total number of compliant turn periods divided by the total number of turn periods. The total number of turn periods can be calculated by dividing the total amount of time an MP is monitored by the MP's turn period interval, with the resultant quotient rounded to the nearest integer value. Alternatively, the turn period intervals can be defined based on predefined time intervals (i.e. 2 am-4 am, 4 am-6 am), or by predefined turn schedule times for the institution, or by any other method. To further illustrate, consider an example where the number of turn periods is calculated based on dividing the total amount of time an MP is monitored by the MP's turn period interval. If this MP is monitored for 24 hours and is on a 2 hour turn period, the MP would have a total of 12 turn periods. If an MP exceeds their turn period in any of those 12 turn periods, they would be considered “non-compliant” during each of those turn periods. Therefore, if an MP is non-compliant in 4 of the 12 turn periods, the overall compliance rate would be 8/12=66%. It should be noted that the turn period intervals could also be reset following an adequate turn.

Compliance (% Turn Periods)=Total # of Compliant Turn Periods Total # of Turn Periods

4. Compliance (% Turn Periods)+Grace Period:

Compliance can be calculated as a function of the total number of compliant turn periods divided by the total number of turn periods, with the incorporation of a grace period. A grace period can optionally be incorporated into the compliance calculation, such that the calculated compliance is not negatively impacted until the grace period is surpassed. A grace period provides a small amount of time for nurses to perform a turn after a turn period has expired, whereby if the turn is completed before the grace period expires, the compliance is not negatively affected. The total number of turn periods can be calculated by dividing the total amount of time an MP is monitored by the MP's turn period interval, with the resultant quotient rounded to the nearest integer value. Alternatively, the turn period intervals can be defined based on predefined time intervals (i.e. 2 am-4 am, 4 am-6 am), or by predefined turn schedule times for the institution, or by any other method. To further illustrate, consider an example where the number of turn periods is calculated based on dividing the total amount of time an MP is monitored by the MP's turn period interval. If this MP is monitored for 24 hours and is on a 2 hour turn period, the MP would have a total of 12 turn periods. In order to get credit for turning during any given turn period, a turn must be completed before the grace period expires.

Compliance (% Turn Periods)+GP=Total # of Compliant Turn Periods Total # of Turn Periods

In addition to compliance, another metric for assessing how closely a turning protocol is being followed is the “severity of non-compliance”. The severity of non-compliance is a measure that reflects the severity or magnitude of non-compliance events. The severity of non-compliance, can be calculated in many ways, but one general method is to compute the median amount of time accrued after a turn period expires. Higher values indicate a more severe the degree of non-compliance. As with the compliance metrics, the severity of non-compliance can be calculated for individual MPs or groups of NIPS, and can be analyzed over a variety of time intervals (i.e. hours, days, weeks, months, quarters, etc.).

Another assessment tool provided by the system described herein allows users to measure how long it takes for an MP to be turned once a turn notification/alert is issued. This assessment tool can be applied to individual MPs or nurses, or groups of MPs or nurses. The assessment tool can be used to determine workforce needs and serve as a measure to track workforce improvement or identify areas of improvement.

Another assessment tool provided by at least some embodiments of the system described herein allows users to measure the efficiency of MP-turning efforts. The efficiency metric captures the degree to which caregiver-assisted turns are performed when clinically warranted or necessary. An “unnecessary caregiver-assisted turn” is any caregiver-assisted turn that is considered clinically unwarranted given its temporal proximity to an adequate MP self-turn. The degree of temporal proximity required in order for a turn to be considered unnecessary can be configured. For example, if a caregiver turns an MP 20 minutes after an adequate MP self-turn, that caregiver-assisted turn could be considered unnecessary. As the percentage of unnecessary caregiver-assisted turns increases, the institutional turning efficiency decreases. A turning efficiency of 100% means that every caregiver-assisted turn was clinically warranted. A lower turning efficiency indicates that nursing resources are not being used efficiently and could be re-allocated more effectively based on individual MP needs. In order to measure turning efficiency, there needs to be a means for differentiating between MP self-turns and caregiver-assisted turns. Several methods can be used to identify caregiver-assisted turns. In one embodiment, caregiver-assisted turns can be manually entered into the system through a user interface, or noted in the system by interacting with the MP sensor (i.e. double tapping the sensor), or by separately documenting caregiver-assisted turns in a logbook or other electronic medical record. Using these methods for documenting caregiver assisted turns, or any other acceptable method, the turning efficiency can be calculated by the following equation:

Turning efficiency (%)=(Total # of caregiver−assisted turns)−(Total # unnecessary turns) Total number of caregiver−assisted turns.times.100

The system, methods, and devices described herein can be used to measure turning protocol compliance, severity of non-compliance, and other performance metrics. The system can track and issue a number of reports that can be used to aid a facility in workforce management, efficiency improvement, and outcomes improvement. The system can provide reports for any given MP, group of MPs, unit, facility, or group of facilities. The system can provide reports over any specified time frame. The system can also take in other data regarding workforce staffing (such as caregiver schedules and shifts), to determine which caregivers or group of caregivers are compliant and where there is need for improvement or more staffing. The system can take into account different staff types and training levels to help determine which level of staff is optimal. This data can be used to help improve the efficiency of the system, by determining necessary staffing levels and identifying where the available staff can be deployed most effectively.

Some embodiments of the system described herein can be used to track the MP census size, and also monitor characteristics of the census, such as the average pressure ulcer risk level, etc. Metrics such as the average pressure ulcer risk level of the census, ambulation level, Braden score, isolation MPs, and other MP characteristics can be used to determine required staffing numbers and ratios. These metrics can also be correlated to turning compliance data, severity of non-compliance data, pressure ulcer incidence rates, and other statistics to determine what impact these metrics have on MP outcomes. The system is also able to track events, such as training programs, new hires, audits, conferences, etc. to determine the effect that such events have on compliance and other statistics.

MP Management Software/Turn Management Software

A user interface to the system is provided by the Turn Management Software. The Turn Management Software accesses the SQL database, analyzes the data, and displays in near real-time the relevant information. Caregivers using the system can customize a turn protocol for each MP being monitored. The software alerts caregivers when an MP has been in an orientation for a duration longer than was specified by the individualized turn protocol.

After an MP Sensor has been applied to an MP for whom monitoring is desired, the Turn Management Software can be used to easily assign the sensor to the MP. During the assignment process, the MP's name, electronic medical record number, and bed can be entered. Additionally, the maximum time an MP is permitted to be continuously on a side may be modified if the default value is not desired. If there is a side of the MP's body that the MP should not be on because of an existing wound or other medical issue, the restricted side may be entered. Upon completing the assignment, the Turn Management Software will display whether the MP is on his back, left side, or right side, and when the next turn is required, and additional information including if the MP is prone or upright. If an MP completes an adequate self-turn, credit is given for the turn and the turn period is reset.

There are additional advantages to using the MP Monitoring System. Since both MP self-turns and caregiver-assisted turns are logged by the system, there is no need to provide assistance to an MP that has recently self-turned, and therefore no alert is issued. This saves the caregiver the time and energy of having to perform a turn that is clinically unnecessary, given that the MP is adequately turning on his or her own. In such a fashion, the system reduces the overall number of unnecessary caregiver-assisted turns. Further, the system makes it financially practical to place a greater fraction of MPs on turn protocols. This is because MPs that have moderate to good levels of mobility will likely perform unassisted more frequently. Placing MPs with greater mobility on a turn protocol administered by the MP Monitoring System does not burden caregivers to the extent that a manual system would. Using the MP Monitoring System, caregivers are freed of having to manually monitor MP orientations, and caregiver attention is required only when an MP fails to adequately self-turn. In such a fashion, caregivers are able to focus their attention on those who need it most, while at the same time ensuring that no MP is neglected.

The user interface is designed to show the status of any MP in the facility. The Turn Management Software allows the caregiver to select and change the ward or unit of interest. This makes the system appropriate for single wards/units, complete hospitals, or even hospital chains. Additionally, reports may be generated showing the turn history of MPs throughout the facility that is being monitored by the system, or any fraction of the facility. The system can track the turning protocol compliance (and other metrics) for individual wards/units, an entire hospital, or groups of hospitals.

Depending upon the embodiment, different permission levels are assigned to various users, for example by IT staff, to give nursing administrators the ability to change the default turn periods that caregivers are ultimately able to select. In some embodiments, administrators can also change the default decompression time—the time duration any side should be “off loaded” in order to fully reperfuse a region of the body. The threshold angle through which an MP must be turned for the system to recognize the change in orientation as a turn, and the threshold angle that defines if an MP is upright can also be configured by a user with appropriate privileges. The ability to generate and view reports is also enabled or restricted based on configurable user permissions.

In an embodiment, the installation of the MP Monitoring System uses and application virtualization product, such as Citrix Xenapp, to serve the Turn Management Software to virtually all common desktop or mobile computing devices, including Microsoft Windows and Apple desktop systems, iPads, and android tablets. A “thin client” such as a web browser is all that is necessary on the caregiver's device. In this way, the caregiver experience can be easily controlled through tools provided by Citrix. A dedicated server computer having Citrix Xenapp installed can be configured and provided by Leaf Healthcare, Inc. or the Turn Management Software can be installed on an existing Citrix system already in use at the caregiver's facility. Alternatively, the Turn Management Software is a native Microsoft Windows application, and can be run natively on Microsoft Windows computing devices. Depending on how the system is configured, users are authenticated by either the Citrix logon process or alternatively by logging onto the windows machine on which the application is running. In installations that use Citrix Xenapp, for example, data security is ensured by encryption performed by Xenapp. Alternatively, if the Turn Management Software is running natively on a caregiver's computer, then MP specific data will be transferred between the SQL database and the caregiver's computer and appropriate measures should be taken by the hospital's IT staff to ensure security of MP information.

Tools provided by Citrix enable the facility to configure Xenapp with the appropriate level of computing resources necessary to achieve the level of reliability and redundancy desired by the healthcare facility.

The high degree of configurability of the MP Monitoring System gives the IT and nursing staff of the facility in which it is used the flexibility to decide how best to implement the system. A status board and/or computing device may be positioned at the central nursing station to give caregivers access to the Leaf User Interface. Additionally, a display device providing caregiver access to the Leaf User Interface can be positioned in each MP's room. The use of mobile computing devices such as laptops or tablet computers can enable dedicated turning teams to easily check which MPs are due or soon due for an assisted turn.

The following section describes the specific elements of the MP Monitoring System:

MP Data

The system stores, in one or more data structures, data associated with the an MP taken from a list including: first name, last name, full name, date of birth, year of birth, month of birth, day of birth, medical record number, MP identifier, date of admission, time of admission, date of discharge, time of discharge, indicator of whether MP is actively monitored, indicators of pressure ulcer risk, Braden score, Norton score, MP's turn period, areas to avoid pressure, locations of pressure ulcer(s), locations of wounds, age, weight, ambulatory status, fall risk, indicator of whether MP has one or more pressure ulcers.

Facility Data

The system stores, in one or more data structures, data associated with the facility taken from a list including: number of wards, number of rooms, names of wars, names of rooms, types of wards, types of rooms, number of beds, types or beds, names of beds, room identifiers, ward identifiers, bed identifiers, indicators of whether a room, ward, or bed are active or occupied.

Settings Data

The system stores, in one or more data structures, data associated with settings for a system implementation taken from a list including: turn period, default turn period, available turn periods, selected turn periods, pause interval, default pause interval, available pause intervals, selected pause intervals, decompression interval, default decompression interval, available decompression intervals, selected decompression intervals, turn angle, default turn angle, available turn angles, selected turn angles, upright angle, default upright angle, available upright angles, selected upright angles, grace period, default grace period, available grade periods, selected grace periods, capacitive threshold, default capacitive threshold, available capacitive thresholds, selected capacitive thresholds, a threshold for the number of consecutive attached entries for a sensor to be considered attached, a threshold for a the number of consecutive unattached entries for a sensor to be considered unattached, a time indicator for the amount of time that the home screen has not been updated before informing a user that the home screen has not been updated.

Turn Angle

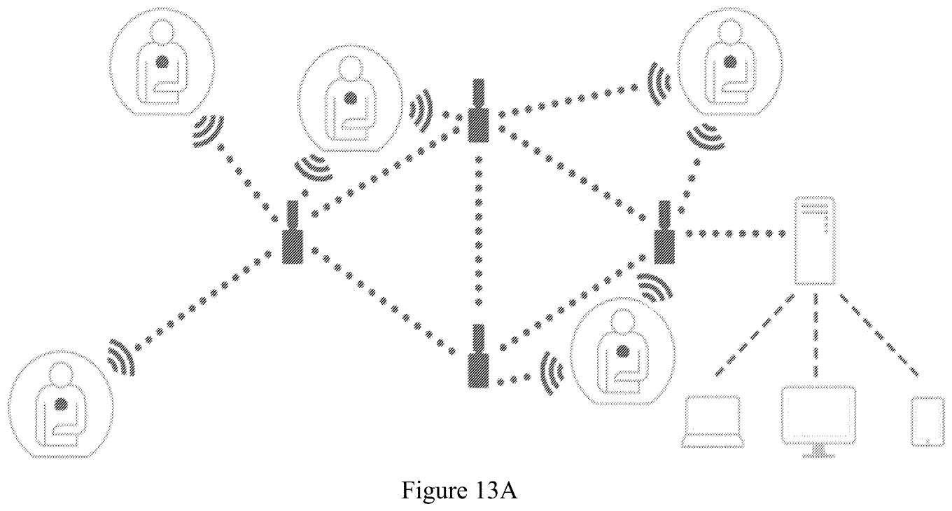

In at least some embodiments, the system has threshold settings at which the MP is considered to be in certain orientations. FIG. 18 illustrates the manner in which left-right turns about the cephalo-caudal axis are assessed. For example, FIG. 18 starts at step 1800 with an MP determined by the system to be laying on their back. As indicated at 1805, the MP rotates, either on their own or with the aid of a caregiver, left about his/her cephalo-caudal axis. A determination is made at step 1810 as to whether the MP rotated far enough left to go past the predetermined “Back-to-Left-Turn” threshold angle. This angle threshold is the back-to-left turn angle. If so, the MP has moved from lying on his/her back toward the left side and is now considered to be on his/her left side, as indicated at 1815. Similarly, the back-to-right turn angle is the threshold angle at which the MP is considered to be on his/her right side. The back-to-right and back-to-left threshold angles are clinically independent, and may be the same or different. Similarly, if the MP is on his/her left side and turns towards the back side, as indicated at 1820, the angle threshold at which the MP is considered to be on his/her back side is the left-to-back turn angle. As before, a determination is made, for example based on sensor data, that the left-to-back threshold angle was exceeded, and so the MP is now identified as being on his/her back, as shown at 1825.

Note that the left-to-back turn angle does not need to be the same as the back-to-left turn angle. Some embodiments of the system can include hysteresis by allowing for different angles for the left-to-back and back-to-left turn angle thresholds. For example, in the case where the left-to-back turn angle is closer to the MP being on the back and the back-to-left turn angle is closer to the MP being on the right, the system may avoid rapidly switching characterizing the MP as being on the back and left side. The difference between the left-to-back and back-to-left turn angles may be set as a constant difference, a ratio, a function, and it may depend on the MP, MP characteristics, MP turn characteristics, or facility characteristics.

A similar relationship exists between the right-to-back and back-to-right turn angles and the right-to-prone, prone-to-right, left-to-prone, prone-to-left turn angels. Depending upon the embodiment, the system can set these threshold turn angles, or they may be set by a facility or by a caregiver or other user. The settings can be simplified in some embodiments such that some or all of the turn angles are symmetric and as few as one angle is needed to be selected. Alternatively, as noted above, each the angles can be selected independently. The settings may be for a facility, a ward, a room, a bed, an MP. The settings can also vary based on past, present, or future data from the facility, caregiver or other user, system, or MP.

Upright Angle

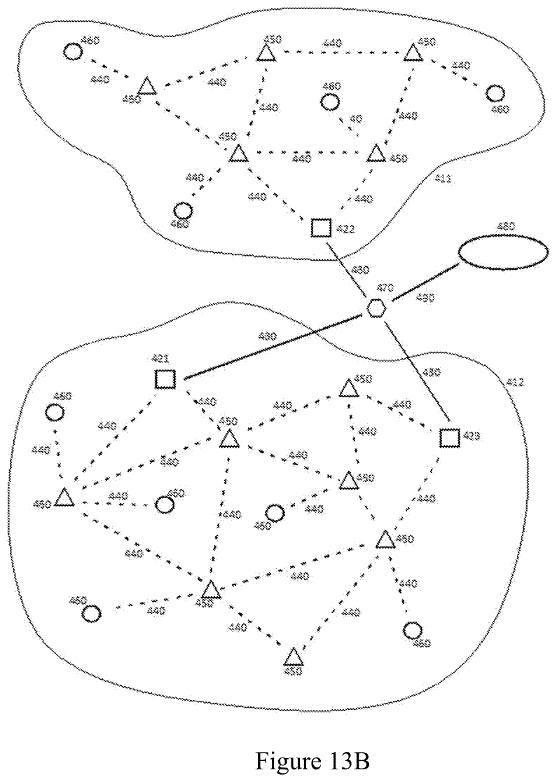

Referring next to FIG. 19, at least some embodiments are configured to detect, through signals from one or more sensors affixed to or associated with the MP, when the MP experiences rotation about his/her left-right axis as shown at 1905. When rotating about this axis from lying on the back to being upright, the MP may pass an upright threshold angle, detected at 1910, at which point the MP is considered to be upright, shown at 1915. This angle can be set by being manually entered by the caregiver or the facility, or can be set or adjusted by the system depending on MP-specific factors or other suitable treatment protocols. The back-to-upright angle defines the angle at which the MP goes from being characterized as lying on his/her back to being characterized as upright. This upright-to-back angle, see 1920, 1925, defines the angle at which the MP goes from being characterized as upright to being characterized as lying on his/her back. The back-to-upright angle may be different from the upright-to-back angle. This hysteresis may be useful to avoid rapidly switching characterizing the MP as being on his/her back to being upright. In one implementation of the system, the back-to-upright angle is more upright than the upright-to-back angle.

Grace Period

In one embodiment, the system allows a facility to set a grace period, which defines within the system a period of time after a turn is due for an MP within which the MP can be turned and no non-compliance event is registered. Stated another way, once the turn period is reached and the MP is to be turned in accordance with a turn protocol, there may be a grace period in which the turn is considered to be compliant with the turn protocol as long as the turn occurs within an amount of time defined by the grace period after the turn period is over. This can be defined by the user or facility and it can be defined for all MPs or set differently for individual MPs based on their needs and turning characteristics. The grace period can also be set individually for a given facility, unit, ward, subset of a facility, hospital system, caregiver, user, or type of MP.

Capacitive Threshold

A capacitive sensor is one of the sensors the system can use on an MP sensor. Among other things the capacitive sensor can be used to detect if the MP sensor is attached to the MP. The capacitive threshold is the capacitance reading at which the sensor is becomes attached or unattached. The capacitive threshold can be set by the system, by the user, or by the facility. The unattached-to-attached capacitive reading threshold can be different from the attached-to-unattached reading threshold. This reduces the rapid switching between unattached an attached near the threshold. In one embodiment, the unattached-to-attached capacitive reading threshold is greater than the attached-to-unattached capacitive reading threshold. The capacitive reading thresholds can also be set individually for a given facility, unit, ward, subset of a facility, hospital system, caregiver, user, or type of MP.

Attached and Unattached Count Threshold

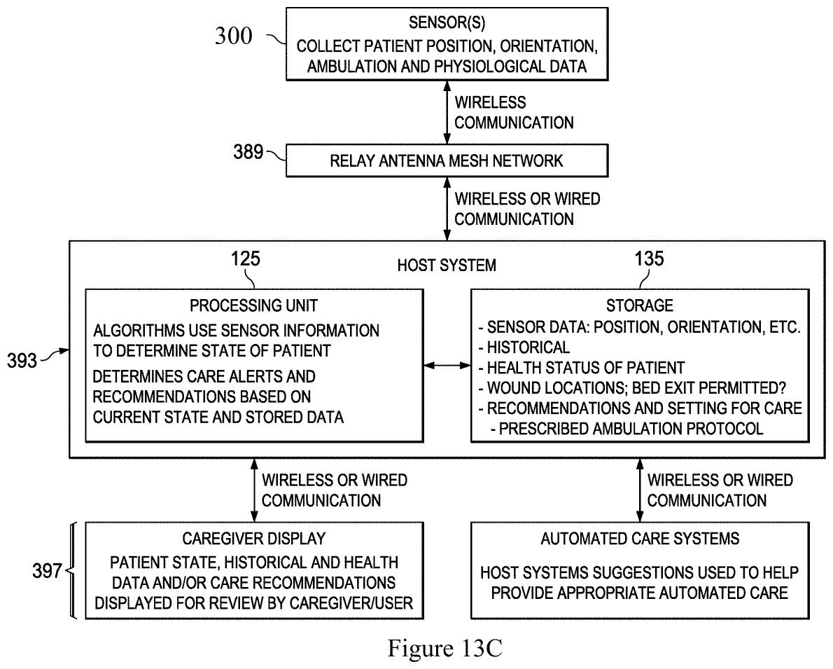

The system may sense whether or not the MP sensor is attached to the MP. In one implementation, the system may use a capacitive sensor to determine attachment, though other sensing methods can be used. As shown in FIG. 20, the system may choose to only determine that the MP sensor is attached to the MP after one or more consecutive attached readings from the attachment sensing, after a certain amount time has passed in which the attachment sensing reports that the sensor is attached, or some combination of a number of readings and time. Similarly, the system may choose to only determine that the MP sensor is unattached from the MP after one or more consecutive unattached readings from the attachment sensing, after a certain amount time has passed in which the attachment sensing reports that the sensor is unattached, or some combination of a number of readings and time. One potential benefit of requiring multiple consecutive readings or period of time in which the sensor showing consistent attachment or detachment is that false attachment reports and transient attachment readings are reduced.

FIG. 20 shows a flowchart for an embodiment of an aspect of the system wherein the MP sensor starts as unattached, at 2000. The system thereafter receives multiple readings from the capacitive sensor and associated attachment logic, which together operate as an attachment sensor within the MP sensor, as shown at 2005 and 2010. The “attached reading” counter in the server increments for each reading, as at 2015. Upon receiving a threshold number of readings indicating that the sensor is attached, see 2020, the system identifies the MP sensor as attached, 2025, and monitors the associated MP.

Warning Time

In the event that one process or thread in the system software fails or stalls, another process or thread that is monitoring it can issue a warning to a user. The system may determine that the process or thread being monitored has failed by periodically checking that the process or thread is working or periodically receiving messages from it. The system may determine that the process or thread being monitored failed or stalled by seeing that there were not messages or that the process or thread has not been running for a certain amount of time and the system may warn the user after this time has passed.

User Group Data

Different user groups may exist that allow access to different sets of data. This separation of user groups allows for simplified management of protected health information and workflow information. Some users may only be allowed to view data that does not contain protected health information. Some users may only be allowed to view data certain MPs. Some users may only be allowed to view information in a particular subset of a facility, such as a ward, or in a particular facility or set of facilities. Some users may not be allowed to view certain sections of the system such as the reports, home page, or settings. Some users may be able to view everything. The system may have preset user groups, and user groups and privileges may be defined by the facility.

Sensor Data

Data that is sent from the MP sensor will, in at least some embodiments, contain no protected health information. Instead, in such embodiments the data contains an identifier that can be associated with the MP to which the sensor is paired. Data associated with the MP sensor can be taken from a list including: time stamp for when the data was received, identifier of the process or user that generated the data entry, time stamp for when the data was generated, the MP sensor identifier, the sensor data including accelerometer data, capacitance sensor data, or attachment sensor data.

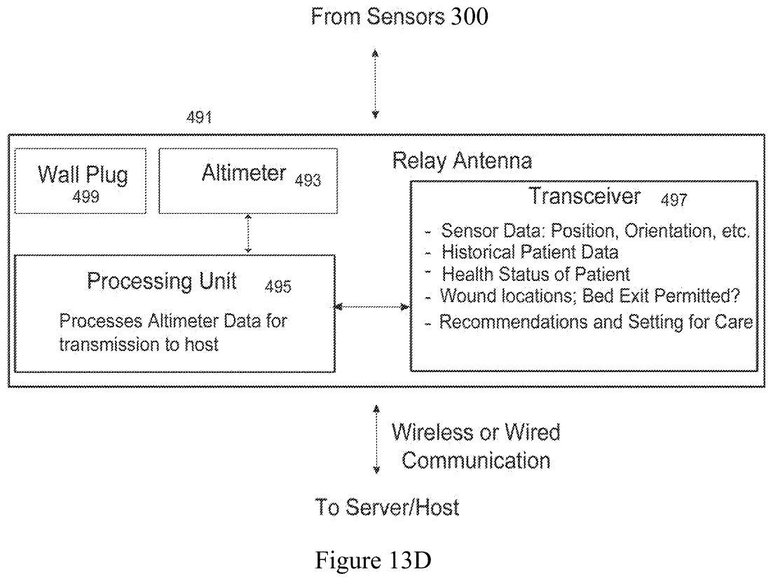

Referring next to FIGS. 21-26, various algorithms which can configure the operation of the host system 335 and which enable prediction and detection of bed exits and/or falls can be better appreciated. Not all of such algorithms need to be used in a single embodiment, and not all of even a single flowchart is required in all instances. In general, there is a characteristic pattern of movement that precedes a bed-exit. Prior to exiting a bed, an MP will typically turn towards the edge of the bed. MPs will also generally sit at the edge of the bed before exiting, unless they exit via a roll maneuver. Upon exiting the bed, there will be characteristic acceleration and altitude changes that occur as the MP moves away from the bed, which can occur in a controlled or uncontrolled fashion. In some embodiments, the pattern of movement that precedes, coincides, or follows a bed-exit can be detected via one or more sensors, including accelerometers, magnetometers, gyroscopes, or altimeters. In some implementations, a characteristic reading from a magnetometer can be used to indicate if an MP is in a position or orientation that is likely to precede a bed-exit. As mentioned, prior to exiting a bed, an MP's torso will generally be substantially parallel to the long-axis of the bed. Stated differently, the MP's anterior torso will be oriented towards the edge of the bed, perhaps in a sitting or upright position. Characteristic magnetometer readings, which may be combined with other sensor data, can be used to indicate that a bed-exit may be pending.

For each bed, the compass heading of the bed needs to be known or determined. The compass heading of the bed can be determined in relation to the long-axis of the bed, or in relation to any other defined axis. Once the compass heading of the bed is defined, it is then possible to know how the MP is oriented with respect to the bed. For example, if the MP's compass heading is orthogonal to the long-axis of the bed, this may indicate that the MP is positioned in a direction that typically precedes a bed-exit. If the MP's compass heading is further combined with orientation data (as determined by an accelerometer), it is then possible to know if the MP is sitting in an upright position at the edge of their bed, which may further increase the probability of a pending bed-exit.

In some implementations, the compass heading of every bed, chair, or other support surface is defined in the system. In terms of defining compass headings, each bed may be able to provide magnetometer data directly or a magnetometer may be associated with each bed. Although the physical location of specific beds may change, the compass heading of any given bed will generally be the same at any given location. MP rooms are typically designed such that the bed is oriented in a particular fashion within the room. The process of defining compass headings for each bed can also be done manually. In order to improve the sensitivity of the bed-exit monitoring system, the MP acceleration and magnetometer data can be combined with real-time location sensing. The location of the MP can be determined via triangulation within the system's mesh network of wireless relay antennas, which are placed at defined locations. The direction that the sensor (and thus the MP) is facing can be determined by analyzing the perceived signal strength within the network of relay antennae.

Sensors applied to the MP's anterior torso can have a substantially directional transmission profile. Body tissues, radiopaque coatings, or other factors can attenuate the transmitted signal. As a result, the perceived signal strength varies with the direction of the sensor relative to the receiver (i.e. relay antenna). For example, consider two wireless sensors that are placed equidistant from a relay antenna, where each sensor has a directional transmission profile. Although the sensors are at the same distance from the relay antenna, the sensor that is oriented towards the relay antenna will be provide a higher perceived signal strength.

In order to know how the orientation of a given sensor affects the communication to a relay antenna in terms of perceived signal strength or time of flight, a system calibration step can be performed. When the system is initially installed and the relay antennas are placed in relatively known locations, a calibration step can be completed to determine the communication readings (signal strength, time of flight, etc.) from a sensor at a given location for all possible sensor orientations, or a subset of common orientations. This calibration step can be done with a calibrating unit that simulates the MP (at least in terms of RF or wireless transmission) and rotates through various different orientations. Once an initial calibration step is done, the location of each MP can be determined. Given that the location of each bed can be provided, it is possible to know if an MP's location is coincident with the location of their bed. If the MP's determined location differs from the location of their bed, it can be assumed that a bed-exit has occurred. Furthermore, the direction that each MP is facing can be determined via perceived signal strength or time-of-flight analysis. This information can be combined with magnetometer data from the MP sensor to further define which direction an MP is facing. In general, MPs will face in a characteristic direction prior to exiting a bed. Taken together, the teachings described herein can be used to determine if an MP bed-exit has happened, or is likely to happen.

In some implementations, the compass heading of every support surface is not initially defined, but is rather determined over time. When MPs lie in bed, they typically lie parallel with the long-axis of the bed. If the MP is associated with a sensor that contains both a magnetometer and an accelerometer, and the MP is generally oriented parallel to the long-axis of their support surface, then the compass heading of the support surface can be inferred. As more data is provided at a particular location (even across multiple MPs), the estimation of the compass heading for the support surface can be further refined. It is also possible that the compass heading for the support surface is initially provided manually or automatically, but then the compass heading is further refined based on MP data.

Systems and methods disclosed herein overcome some of the limitations of the prior art by providing an improved means for activating and deactivating the bed-exit alarm mechanism. First, the system can be designed such that whenever an MP enters their bed, the bed-exit alarm mechanism is automatically set without requiring any caregiver input. Any subsequent bed exits, or attempted bed-exits, will then trigger the alarm. If an MP exits a bed with caregiver assistance, the bed-exit alarm can be disabled manually or automatically. With a manual means, the caregiver can disable the alarm by indicating in the system that the bed-exit is allowed. This “ignore bed-exit” indication can be provided via the user-interface or by interacting with the MP sensor, such as by tapping the sensor in a characteristic fashion. In some implementations, the “ignore bed-exit” indication is provided automatically by recognizing that an MP is in close proximity to a caregiver during a bed-exit. For example, if the caregiver is associated with a sensor and the relative distance between the caregiver's sensor and the MP's sensor can be resolved, then bed-exit alerts can automatically be disabled when the caregiver comes within a certain proximity to the MP. When the caregiver is no longer in close proximity to a given MP, the bed-exit alerts can automatically be re-enabled.

In particular, FIG. 21 illustrates in flow diagram form an embodiment of the process flow for determining if an MP bed-exit is likely to occur, a bed-exit has occurred, and/or a fall has occurred. Beginning at step 2100, an MP to be monitored for bed exits and/or falls is identified in the system, either by analysis of MP characteristics, MP health, MP history, manual entry, or other means. Data is collected from the sensor at 2105 and analyzed at 2110, to determine whether the sensor is operating properly. At 2115 a check is made to ensure that the sensor is functioning properly. If not, a caregiver is notified.

If the sensor is functioning properly, for the embodiment illustrated the process advances to step 2125, the sensor data is analyzed to determine MP orientation, compass heading, location and altitude. Stored data from 2130 is compared to sensor data at 2135, and at 2140 a determination is made as to whether a bed exit has occurred or is likely to occur, or a fall has occurred or is likely to occur. Probability thresholds derived from caregiver input 2145 or algorithmically 2150 are set at 2155 and then compared to the sensor data at 2160, leading to a yes or no conclusion at 2165 as the result of the threshold comparison. If the resulting probability is not greater than the threshold, the system loops via 2170 back to 2125. If the probability is greater than the threshold, a caregiver or automated system is notified directly or via an alarm 2175, 2180, or other means.

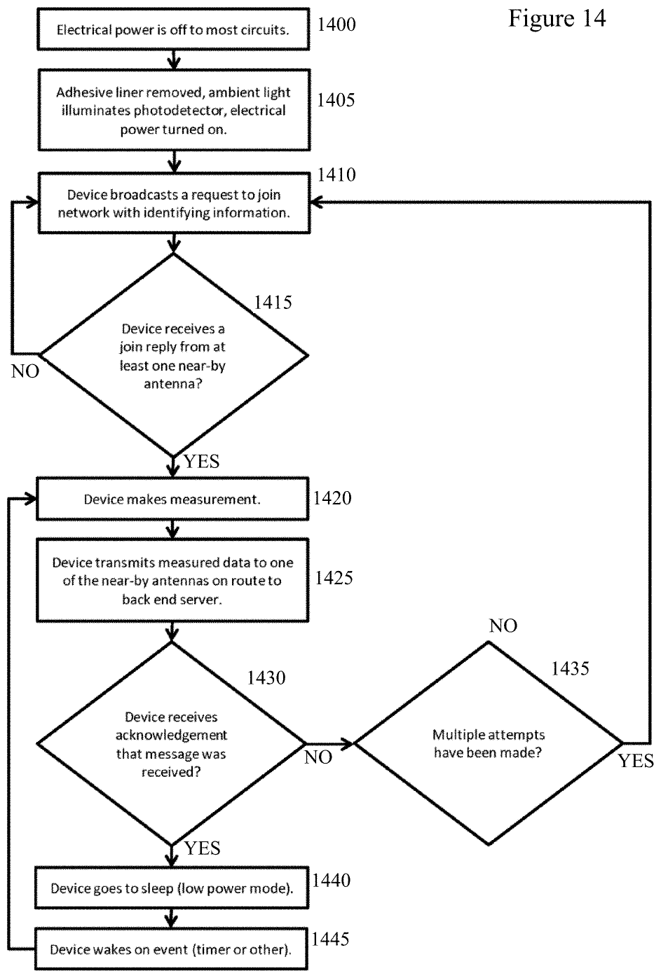

FIG. 22 illustrates in flow diagram form an embodiment of the process flow for determining if an MP bed-exit is likely to occur. At Step 2200, the compass heading for each MP bed is entered into the system. The process of entering the compass heading for each MP bed can occur manually or automatically. In some implementations, users define the compass heading for each bed during a system configuration setup process. In some implementations, the compass heading of the bed is automatically recorded by the bed and communicated to the system. In some implementations, a sensor is applied to the bed such that the compass heading of the bed can be measured and communicated to the system.

In some implementations, the compass heading of each bed is not initially defined, but is rather determined over time. When MPs lie in bed, they typically lie parallel with the long-axis of the bed. If the MP is associated with a sensor that contains both a magnetometer and an accelerometer, and the MP is generally oriented parallel to the long-axis of their support surface, then the compass heading of the support surface can be inferred. As more data is provided at a particular location (even across multiple MPs), the estimation of the compass heading for the bed can be further refined. It is also possible that the compass heading for the support surface is initially provided manually or automatically, but then the compass heading is further refined based on MP data.

At Step 2210, a sensor is applied to an MP's body in a known orientation with respect to their body, where said sensor contains at least a 1-axis accelerometer and a 2-axis magnetometer. At Step 2220, the accelerometer is at least a 1-axis accelerometer, where the longitudinal axis of the MP's body is parallel to the axis of sensitivity of the accelerometer. In some implementations, a 2 or 3-axis accelerometer may also be used, but this is not required in all embodiments.

At Step 2230, a determination is made as to whether the MP is sufficiently upright to be consistent with a bed-exit maneuver. The upright angle threshold can be set at any angle, such as >80 degrees. As the upright threshold angle is decreased, the sensitivity of bed-exit detection may increase but the specificity may decrease (more false positives). At Step 2240, the magnetometer is at least a 2-axis accelerometer, where the longitudinal axis of the MP's body is perpendicular to the plane of sensitivity of the magnetometer. In some implementations, a 3-axis magnetometer may also be used, but this is not required.

At Step 2250, a determination is made as to whether the MP's left-right body axis is sufficiently parallel to the long-axis of the MP's bed. The left-right body axis angle threshold can be set at any angle, such as <10 degrees from parallel. As the body axis angle gets further from parallel with the angle of the bed's long-axis, the sensitivity of bed-exit detection may increase but the specificity may decrease (more false positives). Depending upon the result at step 2250, a caregiver is alerted if a bed exit is predicted with sufficient probability, 2270, or the system loops for another time interval, 2280.

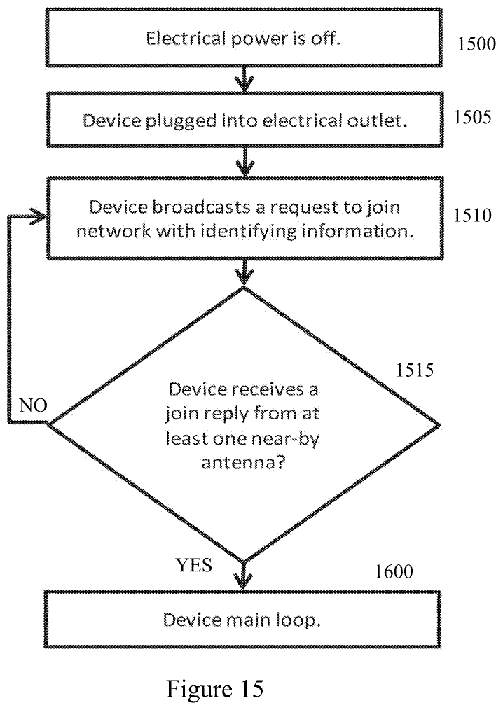

FIG. 23 illustrates in flow diagram form an embodiment of the process flow for determining if an MP bed-exit has occurred using accelerometer and altimeter information. At Step 2300, the altitude of the floor is entered in the system. The process of entering the floor altitude can occur manually or automatically. In some implementations, users define the floor altitude in each MP room or MP care location during a system configuration setup process. In some implementations, the floor altitude is automatically measured by altimeters that are at a known height above the floor and this information is communicated to the system.

At step 2310, the sensor is applied to the MP and activated as before. At steps 2320-2330, a determination is made from accelerometer data whether the MP is in a recumbent position. Next, at steps 2340-2350, an altimeter measurement is taken from the sensor and a determination is made as to whether the MP's altitude is sufficiently close to the altitude of the floor, using the altitude of the relay antennae as a reference. The altitude differential that is required to consider the MP altitude and floor altitude sufficiently close can be defined in the system. As the altitude differential is increased, the sensitivity of bed exit detection may increase but the specificity may decrease, such that more false positives occur. If the altitude differential indicates a bed exit, an alarm is activated and the caregiver is alerted, steps 2360-2370. If no indication of a bed exit, the system loops to step 2380 and tests again at the next time interval.

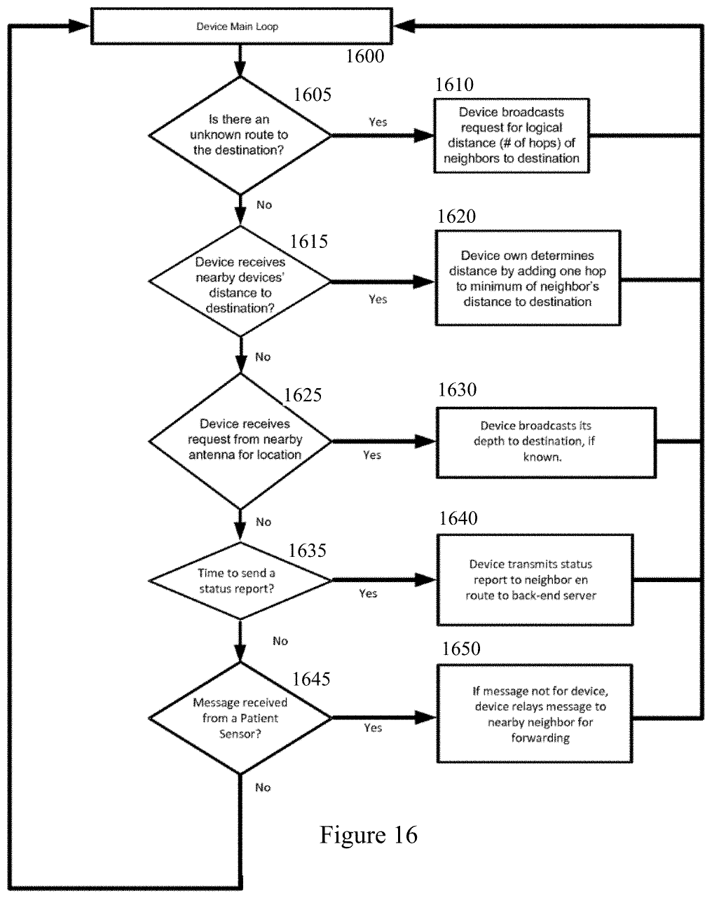

FIG. 24 illustrates in flow diagram form an embodiment of the process flow for determining if an MP bed-exit has occurred using location information. At Step 2400, the location for each MP bed is entered into the system. The process of entering the location for each MP bed can occur manually or automatically. In some implementations, users define the location for each bed during a system configuration setup process. In some implementations, the location of the bed is automatically recorded by the bed and communicated to the system. In some implementations, a bed sensor is applied to the bed such that the location of the bed can be measured and communicated to the system; such a sensor can be the same as or different from the sensor worn by the MP, although attaching a sensor 300 to a bed will cause only a portion of the functionality of the sensor 300 to be used. In any event, a sensor 300 is applied to the MP and activated, as before, and a location measurement is made, steps 2410-2420. At Step 2430, a determination is made as to whether the MP's location is sufficiently close to the MP's bed location to be inconsistent with a bed-exit. The minimum distance required to consider the MP and the MP's bed to be co-located can be set in the system, such as less than two meters. As the minimum distance angle is decreased, the sensitivity of bed-exit detection may increase but the specificity may decrease (more false positives). As with FIGS. 22 and 23, if the measurements indicate a bed exit, a caregiver is notified via a suitable alarm or indicator.

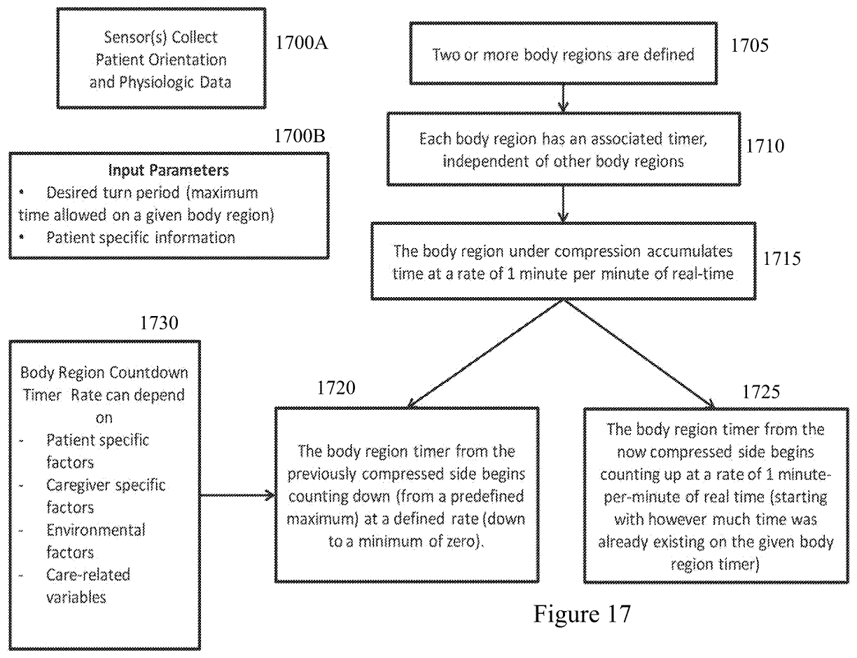

FIG. 25 illustrates in flow diagram form an embodiment of the process flow for determining if an MP fall has occurred using altimeter information. At Step 2500, the altitude of the floor is entered in the system. The process of entering the floor altitude can occur manually or automatically. In some implementations, users define the floor altitude in each MP room or MP care location during a system configuration setup process. In some implementations, the floor altitude is automatically measured by altimeters that are at a known height above the floor and this information is communicated to the system. The MP sensor is applied and activated, step 2510, and an altimeter measurement is made, 2520. At Step 2530, a determination is made as to whether the MP's altitude is sufficiently close to the altitude of the floor. The altitude differential that is required to consider the MP altitude and floor altitude sufficiently close can be defined in the system. As the altitude differential is increased, the sensitivity of fall detection may increase but the specificity may decrease (more false positives). If the sensor altitude is closer to the floor than a permissible differential, an alarm is sent to the caregiver as discussed above, or a loop occurs, steps 2540-2560.

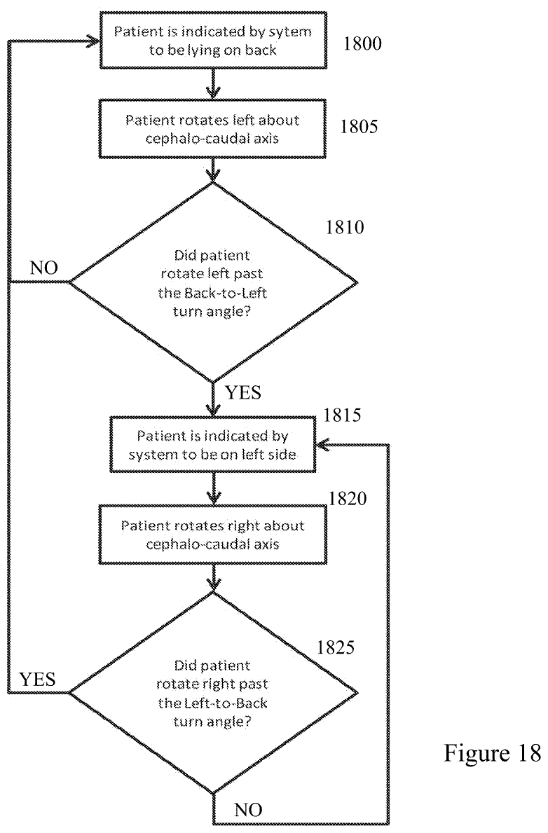

FIG. 26 illustrates in flow diagram form an embodiment of the process flow for determining if an MP fall has occurred using altimeter and accelerometer information. Steps similar to those of FIGS. 21-25 are omitted for simplicity. At Step 2600, the altitude of the floor is entered in the system. The process of entering the floor altitude can occur manually or automatically. In some implementations, users define the floor altitude in each MP room or MP care location during a system configuration setup process. In some implementations, the floor altitude is automatically measured by altimeters that are at a known height above the floor and this information is communicated to the system. At Step 2630, a determination is made as to whether the MP's acceleration is consistent with a fall. The acceleration changes that are required to consider the MP to have fallen can be defined in the system. As the magnitude of acceleration changes is decreased, the sensitivity of fall detection may increase but the specificity may decrease (more false positives). At Step 2650, a determination is made as to whether the MP's altitude is sufficiently close to the altitude of the floor. The altitude differential that is required to consider the MP altitude and floor altitude sufficiently close can be defined in the system. As the altitude differential is increased, the sensitivity of fall detection may increase but the specificity may decrease (more false positives).

In some implementations, the magnetometer is a small, low-power, digital, 3-dimensional magnetic sensor that is responsive to magnetic fields, such as the earth's geomagnetic field. The magnetometer provides a means for determining the direction an MP is facing. When used in conjunction with a 3-axis accelerometer, orientation-independent compass information can be provided. It should be noted that combining a 3-axis accelerometer and a 3-axis magnetometer could simulate the data provided by a gyroscope. In some implementations, the sensor may incorporate a gyroscope, although it would need to be substantially miniaturized in order to be suitable for the intended application.

In addition to the foregoing features and aspects of the system, various user interface features assist in providing an efficient, easy to use, reliable MP management system. Some of these features are discussed below.

Splash Screen

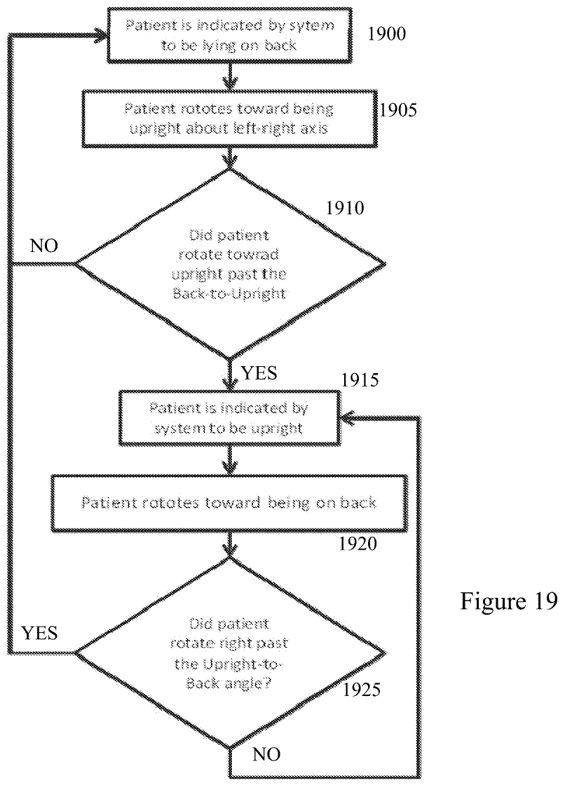

In one embodiment of the system, shown in FIG. 27, a screen appears after the software is started or logged into showing information taken from a list including: Date, Company Logo, Product Name, Client Version, Network Version, Database Version and User Access Level. This screen can appear other times as well or be displayed upon request by the user.

Side Panel

In one embodiment of the system, a side panel is present in the user interface to facilitate navigation. Navigation buttons on the side panel can exist to allow access to different components of the user interface. These buttons along with other features, such as the exit button, help button, user ID, time, date, default turn period, default turn angle, decompression interval, facility name, and unit name, may be present on the side panel to by accessible by the user while accessing one or more or all sections of the user interface.

System Update Clock

In one embodiment of the system, a process or thread within the software is used to update a system update clock. The system update clock is used to show the time to the user in the user interface. The system can use this clock as an indicator to the user that the system is not working correctly if the clock is frozen, shows an incorrect time, or if the time increments at an incorrect pace.

Select Unit Screen

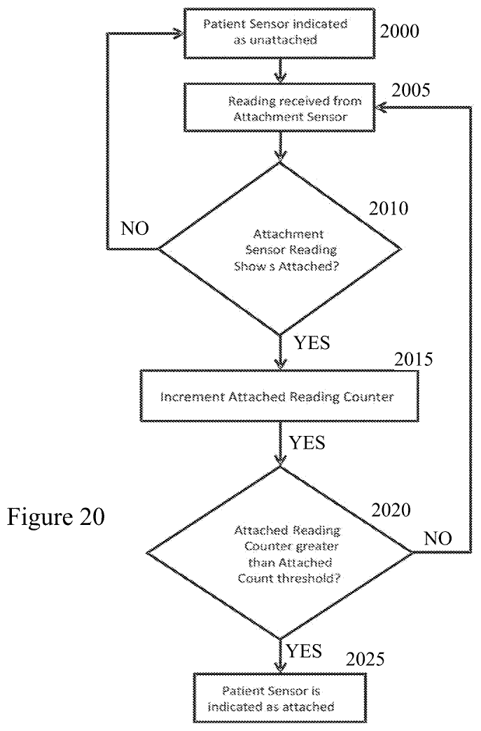

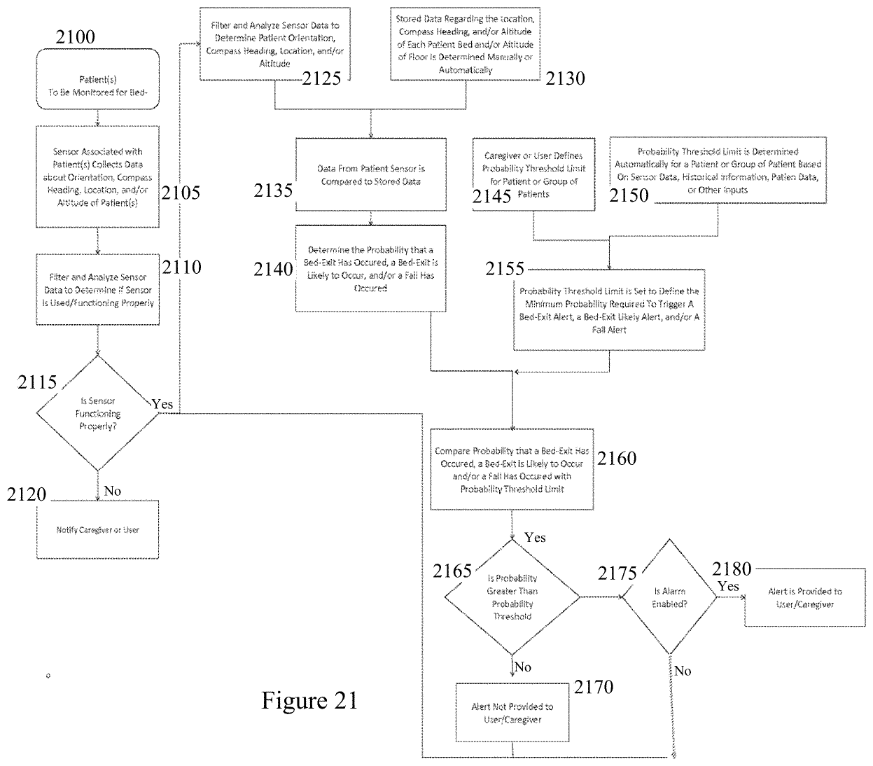

In one embodiment of the system, a section of the user interface exists where the user can select on which facility, unit, ward, or set of MPs they wish to view data, as shown in FIG. 28.

Home Screen

In one embodiment of the system, a section of the user interface exists where the user can view data from one or more MPs. The home screen can be set up to show all MP within a ward or unit or other subsection of a facility. The home screen can be set up to show one or more selected MPs, or a group or type of MPs. The MPs can be sorted by room or bed or location. All rooms or beds can be shown regardless of whether the room or bed is occupied by an MP or an MP being monitored by the system. One potential benefit of this type of display is that a user can look to a constant location on the display for data on a specific room or bed. The MPs can also be sorted by other data including: name, MP sensor identifier, caretaker, risk level, Braden score, time until next turn, turn period. The home screen can display information taken from a list including: room, bed, MP initials, MP name, time until next turn, time on current side, position or orientation, alerts. Graphical representations of the time until next turn, side, room, position or orientation may be used for quick viewing and to meet space constraints. The time until next turn can be displayed as a progress bar, clock, pie chart, or other graph. Different colors in the text, background, or graphics may also be used. The text, background, or graphic may be green when no turn is needed soon, yellow when a turn is close to due, and red when a turn is close to due or due. The home screen can also show how much time has elapsed since the turn was due. The home screen can also have a tag with a certain color with which the user can mark certain attributes associated with one or more MPs. The MPs can be displayed in one or more tables and in one or more columns. The tables may be scrollable. In one embodiment of the system, the system allows users to interact with the home screen by clicking on the MP or information associated with the MP and entering information or opening an MP information window.

Unassigned Sensor Table

In one embodiment of the system, and shown in Figure a table or list of the unassigned sensors is shown in the user interface, either in part of the home screen or in a different part of the user interface. The unassigned sensor table can display information taken from a list including: room, bed, MP sensor identifier, time until next turn, time on current side, position or orientation, alerts. Graphical representations of the time until next turn, side, room, position or orientation may be used for quick viewing and to meet space constraints. The time until next turn can be displayed as a progress bar, clock, pie chart, or other graph. Different colors in the text, background, or graphics may also be used. The text, background, or graphic may be green when no turn is needed soon, yellow when a turn is close to due, and red when a turn is close to due or due. The unassigned sensor display can also show how much time has elapsed since the turn was due. The MPs can be displayed in one or more tables and in one or more columns. The table may be scrollable. In one embodiment of the system, the system allows users to interact with the unassigned sensor table by clicking on the sensor or information associated with the sensor and entering information or opening an unassigned sensor window.

MP Information Window

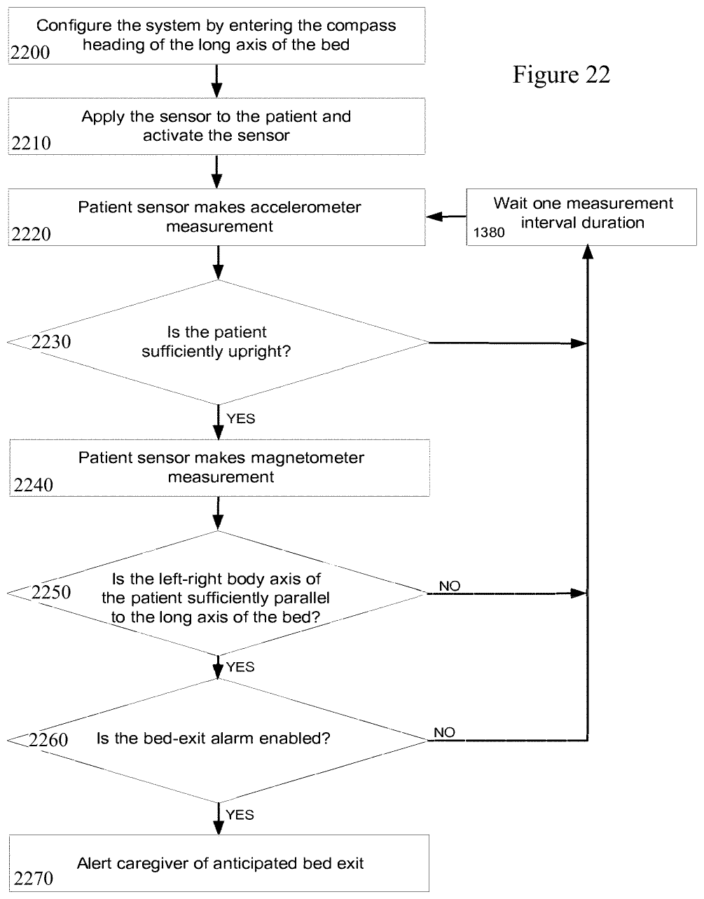

In one embodiment of the system, the system allows the user to click on an MP entry within the home screen to access and edit information about the MP by opening an MP information window, such as shown in FIG. 30. The MP information window can display information taken from a list including: MP sensor identifier, unit, ward, room, bed, tag, MP name, MP first name, MP last name, medical record number, MP identifier, date of birth, restricted areas, areas to avoid, pressure ulcer data, turn period, time until next turn, turn alert pause status. One or more elements of this information can be edited by the user.

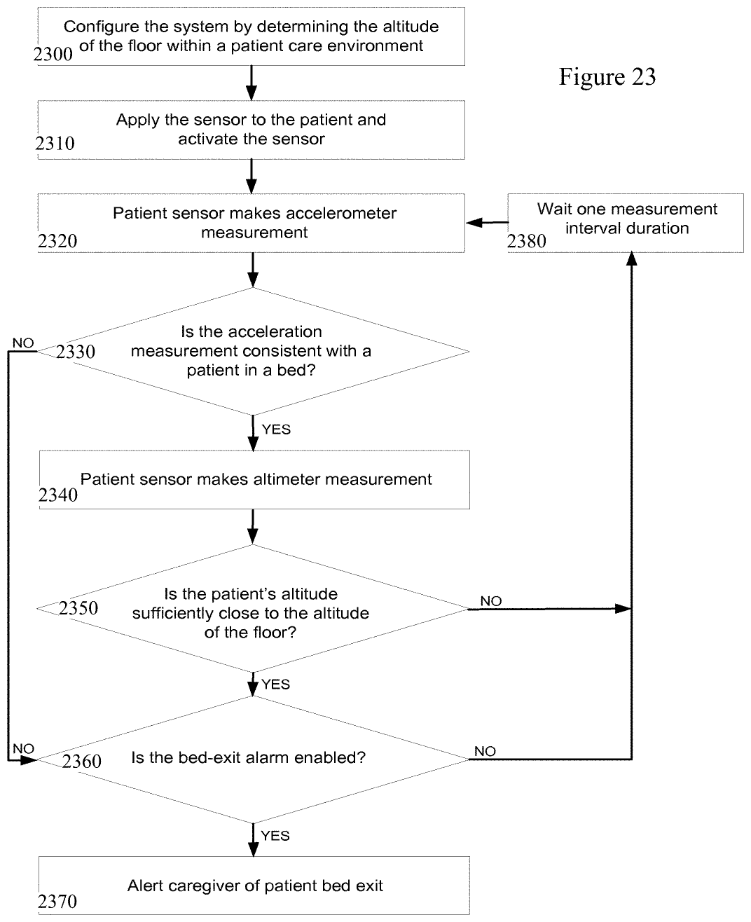

In one embodiment of the system, the system allows the user to click on an MP sensor entry within the unassigned sensor table to access and edit information about the MP by opening an MP information window, such as shown in FIG. 31. Editing this information allows the sensor to be associated with an MP or room or bed. The unassigned sensor window can display information taken from a list including: MP sensor identifier, unit, ward, room, bed, tag, MP name, MP first name, MP last name, medical record number, MP identifier, date of birth, restricted areas, areas to avoid, pressure ulcer data, turn period, time until next turn, turn alert pause status. One or more elements of this information can be edited by the user.

Discharging MP or Deactivating Sensor

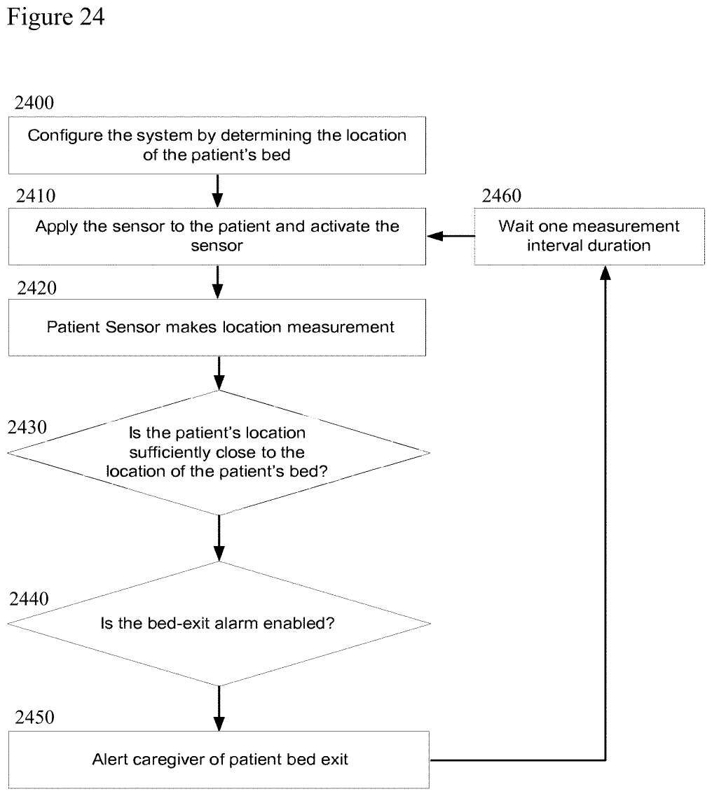

In one embodiment of the system, a window such as FIG. 32 can be displayed when the user requests to discharge an MP. The window can display information about the MP or sensor such that the user can easily verify that the correct MP or sensor has been selected.

Assigning Sensor to Existing MP

In one embodiment of the system, if a user desires to assign a sensor to an MP who is already being monitored, the system can allow the user to select the MP form the home screen and assign the sensor to that MP, reducing the amount of data entry needed to assign the sensor to the MP. This can be useful when switching sensors on an MP or adding additional sensors to an MP.

Pausing Turn Alerts

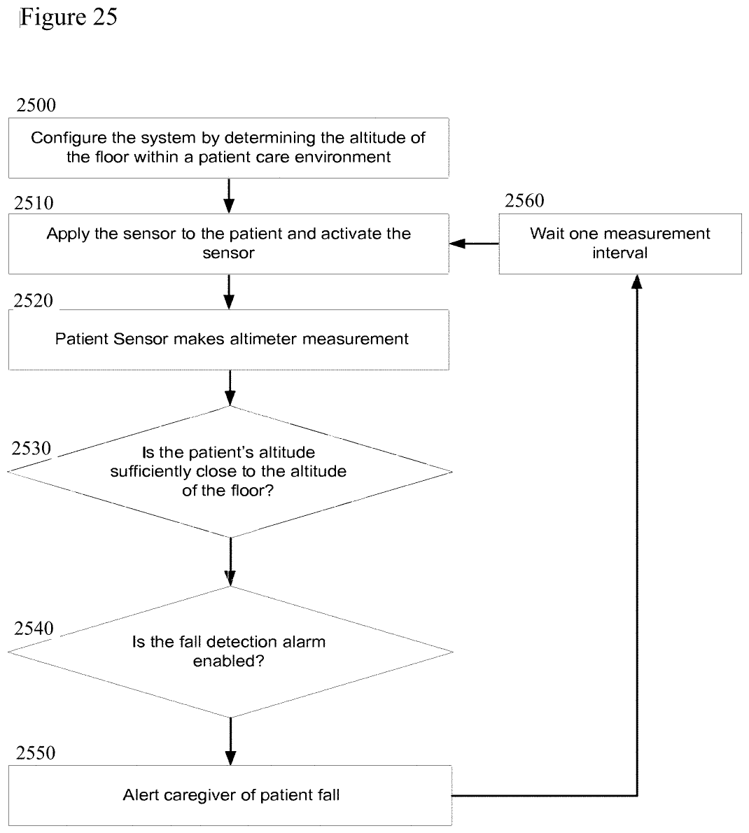

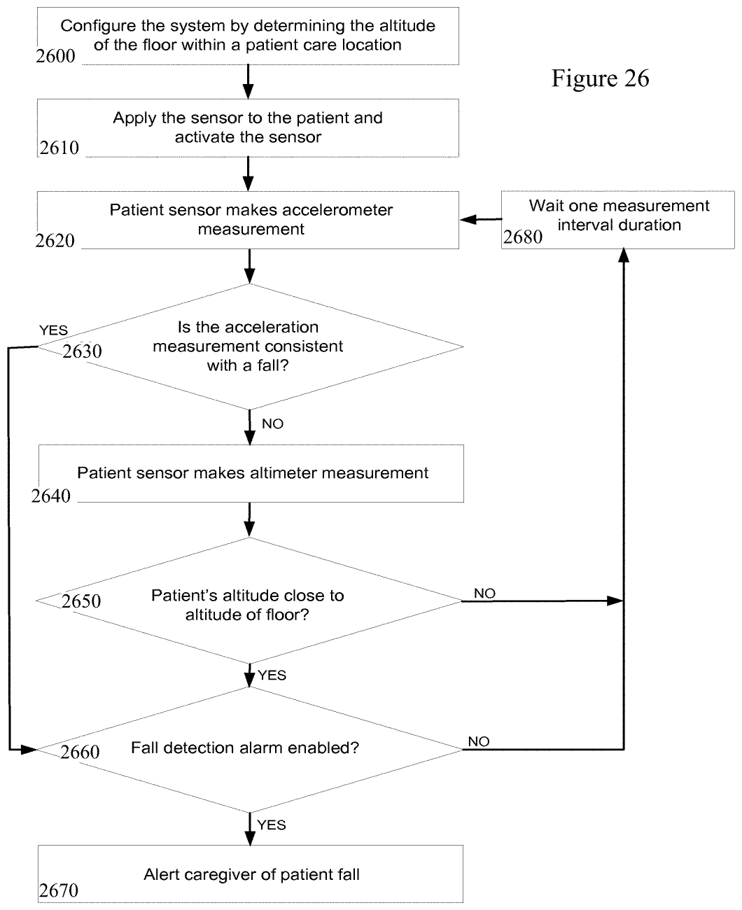

In some embodiments of the system, if the MP has a medical procedure scheduled, the MP has an exam in progress, the MP is out of the bed, or is otherwise unavailable to be re-oriented, the system allows the user to pause the turn alerts and note the reason. A user interface screen for this function is shown in FIG. 33, and the associated system flow is shown in FIGS. 34A-34B.

In one embodiment, the system allows for a user to pause turn alerts and provide a reason why one or more MPs cannot be turned during a certain time period. The user may pause a turn alert in the user interface of the system, as shown in FIG. 33. In one embodiment of the user interface, the selects one or more MPs or rooms for which to pause turns. The user may select from a list of reasons including: MP refuses, MP not in room, MP not in bed, MP undergoing a procedure, MP busy, MP being examined or interviewed, MP is sleeping, equipment interferes with turn, or MP in unstable. The user may also enter a different reason or give no reason. The user may also select the time that the pause is effective for. The user may specify one or more of the following: pause period, pause interval, pause start, pause end, pause start trigger(s), pause end trigger(s). The system can record one or more of the following: the MP, the user, the pause time, pause interval, pause start, pause end, pause triggers, reason(s) for pause. The system may use the pause information to generate data about pause characteristics, users, or MPs. In one embodiment, the system may use the pause information to inform compliance. For example, if the MP was planned to be turned but was not, and a legitimate pause reason was set, then the lack of turn may not be counted as a missed turn or a non-compliance event. The system may end the pause at the time specified by the user, at the end pause interval, when the MP is next turned, or upon certain triggers.

Manually Enter Turn

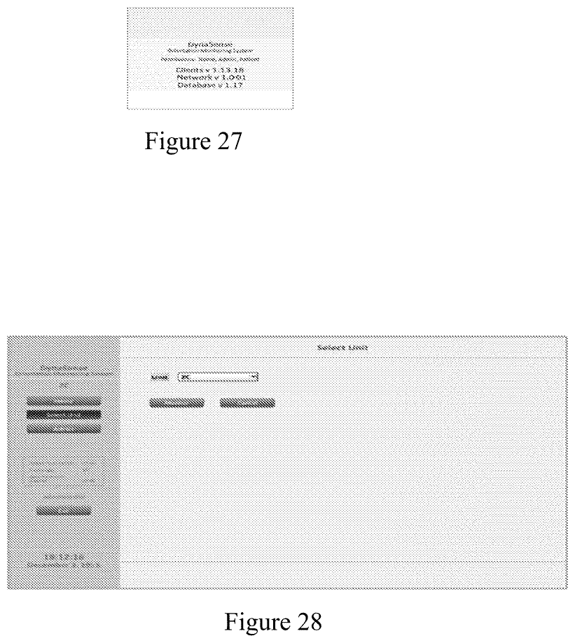

In one embodiment, as shown in the user screen of FIG. 35, the system allows the caregiver or other user to manually enter a turn for an MP, specifying which orientation/position the MP is turned. The system may log the turn as having happened when the manual turn was entered or the user may enter the time of the turn. One instance in which this can be used is when one or more MP sensors or the network is not communicating correctly.

Verify Sensor Attachment

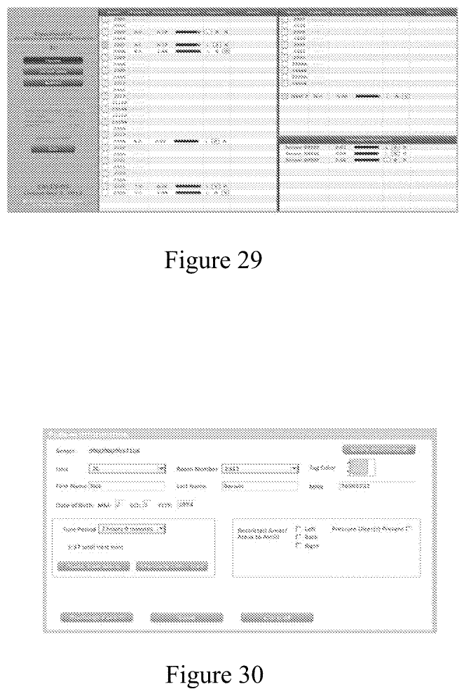

In one embodiment, if the system indicates an MP sensor as unattached, the user can check to see if the sensor is actually attached. A user interface screen depicting such function is shown in FIG. 36. If the sensor is correctly attached and the MP sensor is oriented correctly with respect to the MP, the system allows the user can verify the attachment within the user interface. The user interface show a display of how the sensor should be attached and oriented relative to the MP for reference. The system allows the user to verify that the sensor is attached to the MP and that the sensor is oriented correctly relative to the MP. Once attachment is verified by the user the system records that the sensor is indicated to be attached. If subsequent readings from the attachment sensor show that the sensor is attached or if there are not additional attachment sensor readings from the sensor, the system continues to indicate that the sensor is attached. If the attachment sensor indicates that the sensor is unattached, the system may indicate that the sensor is unattached again. In one embodiment, the system allows the user to ignore future attachment readings from the sensor.

Administrative Settings

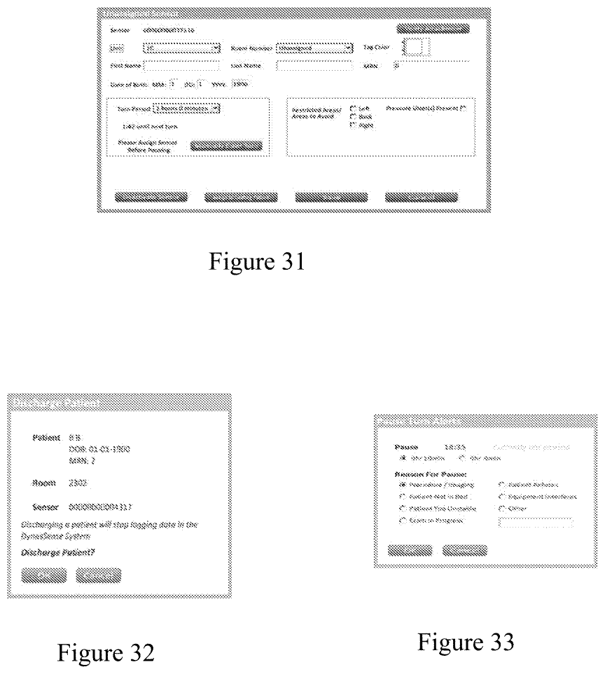

The system allows for users to enter settings for a particular facility, unit, ward, subsection of a facility, group of facility, or group of MPs. Settings include the turn period default and options, turn alert pause interval default and options, the turn angle, upright angle, and the decompression default and options, as shown in FIGS. 37A-37B.

Alerts

In one embodiment, the system issues a number of alerts to the user or facility or for storage in its records.

Database Warning

In one embodiment, the system issues a warning when there is difficulty accessing a database. The system may have a certain threshold of time or attempts to access the database before issuing the warning. The system may also warn when an action cannot be performed due to difficulty accessing a database.

Turn Due Alert

In one embodiment, the system triggers the turn due alert if the accumulated time that the MP has been in a given position exceeds the threshold set by the user or facility. The time that the MP has been overdue for a repositioning may also be displayed. Repositioning the MP can lead to automatic resolution of this alert trigger once the MP Sensor updates its status.

Upright Alert

In one embodiment, the system triggers the upright alert if the angle at which the MP Sensor on the MP's torso is tilted is greater than the threshold Upright Angle. This is not an Alert that inherently requires action. However, repositioning the MP can lead to resolution of this alert trigger.

Prone Alert

In one embodiment, the system triggers the prone alert if the acceleration detected along the MP's anterior-posterior axis by the MP Sensor is negative. This is not an Alert that inherently requires action. However, repositioning the MP can lead to resolution of this alert trigger.

No Signal

In one embodiment, the system triggers the no signal alert if data from the MP Sensor has not been detected by the User Interface to have been updated within a time threshold. The user may be instructed to determine if the MP Sensor is too far from any Relay Antennas or if something may be blocking the signal. Alternatively, the user may replace the sensor or contact Centauri Medical Customer Service

Unattached

In one embodiment, the system triggers the unattached alert if the attachment sensor, which can be but is not limited to a capacitance sensor, reports a reading beyond the threshold for human attachment.

Pause Alert

In one embodiment, the system triggers the pause alert when the user sets a Pause Turn Alert for an MP. This alert can last for a duration equal to the duration of the pause. This is not an Alert that inherently requires action. The alert may disappear when the pause has expired.

MP Information Not Updating Warning

In one embodiment, the system triggers the MP information not updating alert if the database has not been accessible for greater than or equal to a preset threshold, and the time elapsed since the database had last been accessible can be displayed. The user should be aware that the data displayed may be out of date.

Home Screen Warning

In one embodiment, the system triggers the home screen alert if every MP and MP Sensor displayed on the Home screen has not been updated over a period equal to a preset threshold, and the database is accessible, and the duration since the last complete update can be displayed. This can tell the user that the data displayed may be out of date by the amount of time in the preset threshold.

Indicators on Sensor

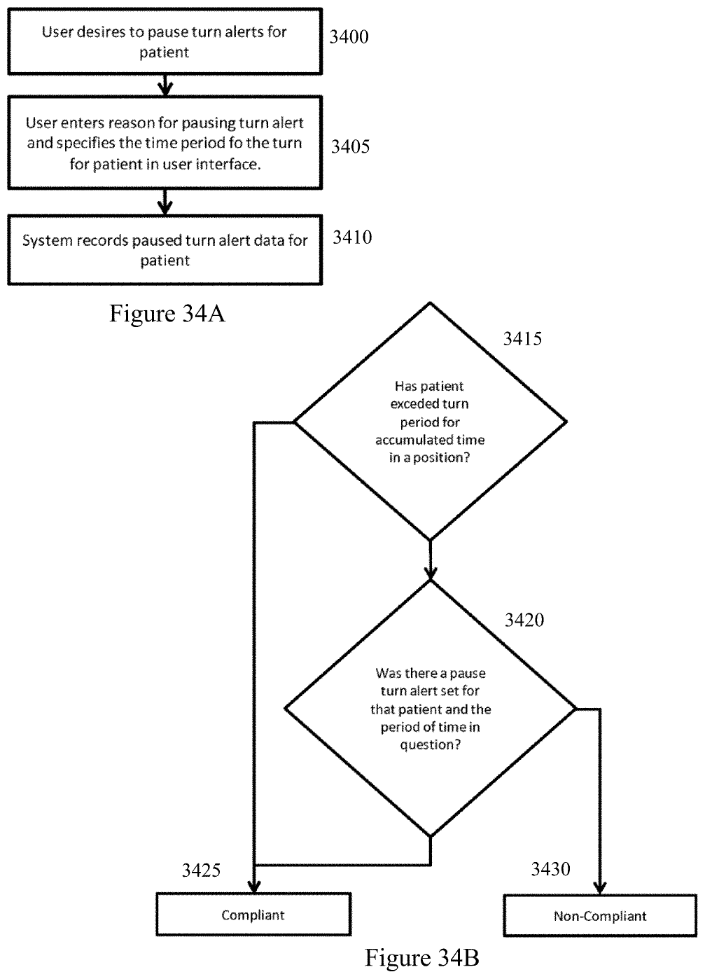

As shown in FIGS. 38A-38B, in some embodiments there may exist indicators 3850 on the sensor, for example LEDs, which give information about the MP. These indicators may indicate which side the MP is on, when an MP requires a turn, which area of the body has been exposed to the most pressure, which direction an MP should be turned onto, or when an MP has been turned sufficiently to satisfy a turning protocol or to depressurize a given area. For example an LED on the left side may turn on when the MP is on their left side. Similarly, in another implementation or setting, the LED may be used to indicate when an MP should be turned and in which direction an MP should be turned. The LEDs may also indicate the relative pressurization levels at different body regions.

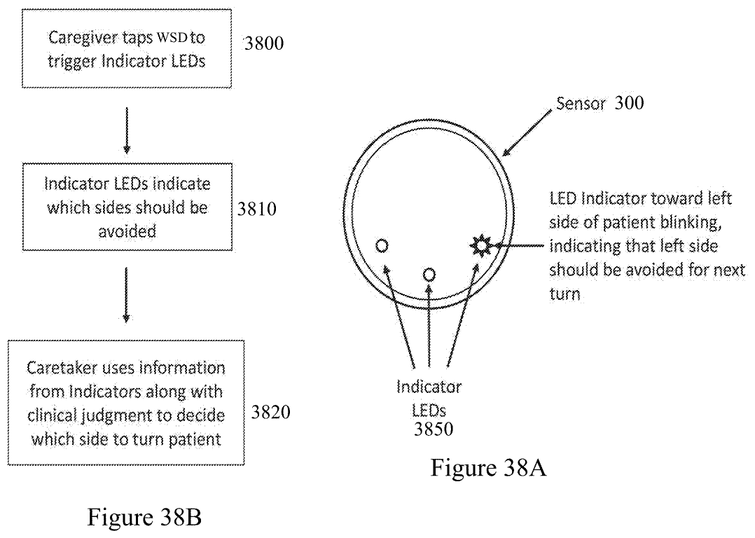

In some embodiments of these sensor indicators, the indicator may be displayed only when triggered. Triggering, as opposed to being on constantly or periodically, can allow for reduced battery consumption and reduced light pollution. In some embodiments, the caregiver may provide the trigger as shown at 3800. The trigger may take the form of one or more of a single tap or sequence of taps on the sensor as discussed hereinafter in connection with FIG. 40, exposure of the sensor to given threshold of light, a switch or button on the sensor, or a wireless communication (which may include RF, sound, light) to the sensor, in response to which the indicator LEDs indicate, for example, which sides should be avoided as shown at 3810, allowing the caregiver to make clinical judgments at 3820. In one implementation of the light threshold trigger, the threshold of light would be exceeded when the caregiver lifts the sheets or clothing to view the sensor, and the LEDs would then come on. The wireless communication can be provided by the caregiver, either by sound or wireless communication generation. In one implementation of the wireless communication, the caregiver can carry an RF transmitter that transmits a signal to the sensor when the caregiver is near or when the caregiver presses a button on his/her transmitter. The transmitted signal causes the indicators on the sensor to display.

MP Self Roll

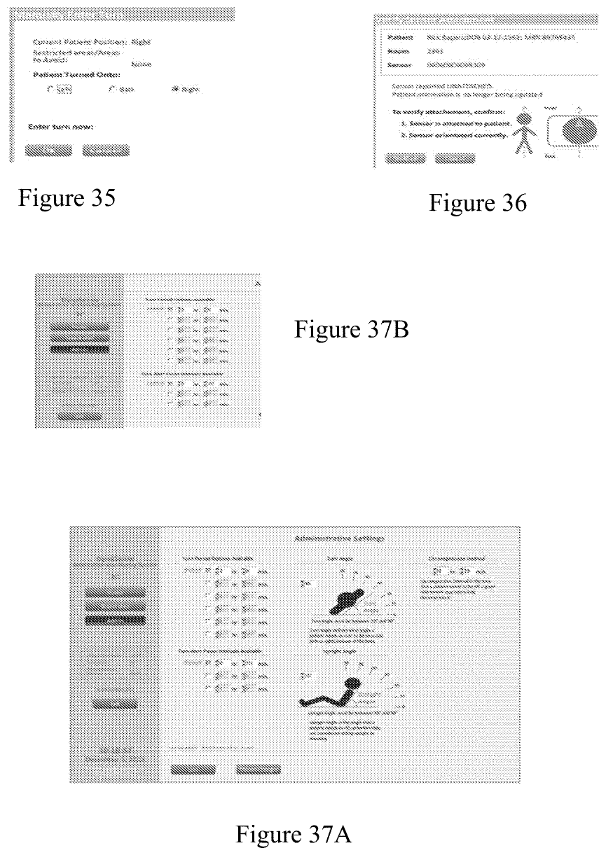

MPs can often reposition themselves to some degree. As shown in FIGS. 39A-39B, the system may be used to encourage repositioning and/or to encourage a somewhat specific direction of repositioning, as shown at 3900. For instance, if it is desired to have the MP reposition onto their left side, 3905, encouragement may be given for the MP to roll onto the left side as shown at 3910. Such encouragement can include:
Audio guidance, which may include voice guidance, the voice of a known person (MP himself/herself, loved one, caregiver, famous person, song, music), or a generated voice
Visual guidance, lights, lights of increasing brightness, lights of varying color or brightness, blinking lights
Noises, beepers, sirens
Physical guidance, including a push, nudge, elevation or angle change of the support surface, a pressure change of the support surface, vibration, tickle, such as via a feather, etc.
Temperature

One or more of these methods can be used in combination either simultaneously or in spatial or temporal relation to one another. Certain stimuli may encourage the MP to turn away or towards without waking up or greatly disturbing sleep. The light or audio or physical stimuli are examples. MPs may naturally turn away from sound, lights, or nudges. In this way an MP may be encouraged to reposition according to a protocol or avoid pressure on certain areas. an MP self-turn reduces the need for caregiver interaction and promotes MP independence. If an MP does not reposition sufficiently, determined at 3915, a caregiver can be notified as shown at 3920. In an embodiment, the stimuli for turning can be external to the MP sensors, such as a unit on the bed, as shown at 3950 in FIG. 39B.

Caregiver Units

In some implementations, the caregiver can also carry components of the system with them. In one implementation, the caregiver has a badge, nametag, bracelet, or other wearable device which is recognized by the system. The caregiver is associated with one or more wearable devices, which each comprises an identifier (such as a name, number, code, etc.). The wearable device wirelessly transmits to base stations that are in known locations. As can be appreciated from FIG. 40, discussed hereinafter, using the methods previously described in the section on “Location Sensing”, the system can determine when the caregiver is in a given room, provide information about when the caregiver is interacting with an MP or other caregiver, or can determine when the caregiver is in any particular location, such as a room, the nursing station, supply closet, or hand-washing area. The location of the caregiver or indication of caregiver-MP interaction can be used to determine when a caregiver helps to reposition an MP. This can be used to determine who is repositioning an MP and to determine if sufficient self turns by the MP are being performed. Caregiver devices can also be used to login to the system when entering information or to help pull up or assign MP information or data related to the MP(s) that the caregiver is assigned to. Devices can be wirelessly charged, passive RFID based, or charged by a physical connection. In one implementation, the devices can be charged inductively by having the device placed in close proximity to a charging unit, such as a charging surface or box. The wearable device can also display or present information visually or audibly. The unit may also indicate when alarms/notices are given. For instance a nurse may be given an audible message or a written message to indicate that a given MP requires turning, or has exited bed, or has fallen. Lights, such as LEDs, may give information, including alarms as well.

The caregiver can also carry a device, such as a handheld reader or scanner. This reader can be used to scan or wirelessly communicate with one or more of MP sensors, a bed or room sensor, an MP ID tag/bracelet, etc. In an embodiment, the device can communicate with a computer or with a sensor or node network or other wireless communication network. In some embodiments, the device can include a barcode reader. In various embodiments, the device can be handheld, attached to a computer, a phone, or a bracelet. The device can also have an audible or visual information display as described above for the wearable device. These devices and the wearable devices can also be used for communication between MPs and caregivers or between caregiver themselves, again as illustrated in FIG. 40, discussed below.

Alternating Pressure Mattress Detection

Acceleration and orientation monitoring of the MP may be used to monitor for motion caused by an alternating pressure mattress. The disclosed monitoring system, by detecting MP accelerations, can determine if an MP is being repositioned sufficiently. Threshold acceleration values can be set, such that if the acceleration threshold is not met in a specified period of time, then it can be assumed that the MP is not being repositioned sufficiently. Alternating pressure mattresses utilize a series of inflatable air cells that inflate in a regular pattern, so as to encourage tissue depressurization of the subject lying on said alternating pressure mattress. This pattern of inflating/deflating air cells will generally cause rhythmic accelerations in an MP lying on the support surface. If no rhythmic accelerations are detected that are consistent with the known pattern of the support surface, then it can be assumed that the alternating pressure mattress is not turned on or is not functioning properly. Specialty support surface actions, which include alternating pressure or repositioning, may cause characteristic accelerations on the body of the MP. For instance, many support surfaces that provide alternating pressure or repositioning do so by inflating or deflating air cells within the support surface. This inflation and deflation is often associated with small vibrations form one or more components of the support surface system, such as a pump or compressor.

Caregiver Interaction with Sensor

In some embodiments, the caregiver can communicate with the disclosed monitoring system by physically interacting with a wearable sensor device (WSD), such as, for example, by tapping as shown at 4000 in FIG. 40. The WSD may include any components and features of the example wearable sensor devices disclosed herein, e.g., including an accelerometer (e.g., 3-axis accelerometer) and associated logic (e.g., software or firmware) for analyzing acceleration data generated by the accelerometer. The accelerometer and software on the sensor can be configured to monitor for taps to the WSD, shown at 4005-4040. In such a manner, a caregiver can tap the WSD to communicate with the system. This communication method can be used to communicate that a caregiver provided a repositioning procedure or that the caregiver is interacting with the MP. A caregiver may also use taps to indicate his or her presence so that the MP sensor can communicate with her via a display, such as one or more LEDs, beeps, or a display screen. The tapping motion causes accelerations/decelerations that can be detected by the WSDs onboard accelerometer. The system can be configured to recognize specific patterns of accelerations/decelerations in order to communicate information with the system. The magnitude of acceleration/decelerations that are considered to be consistent with a “tap” can be predefined in the system. Furthermore, different patterns of successive taps can be used to communicate different information, as at 4035. As such, the WSDs accelerometer functions as an input device for caregivers. For example, if a caregiver wants to inform the system that they are physically present with the MP, they may tap the WSD in a specified pattern, such as two taps at .about.1 Hz. As another example, if the caregiver wants to activate the WSDs onboard LEDs (which can visually display data such as cumulative pressurization time on each side of the body), they can tap the WSD three times at .about.1 Hz. Those skilled in the art will recognize that there are various methods of communicating with the monitoring system via the WSDs onboard accelerometer and associated processing algorithms.

Sleep Monitoring

The system can monitor for characteristic movements associated with different indicators of sleep quality. These characteristics include apnea and movement, activity, or orientation during sleep. Reports can then be given about sleep quality to MPs and caregivers.

Feedback to Support Surface

In some embodiments, the system can include a pressure measurement system which can produce a pressure map of reasonable precision that then feeds back to a support surface. This pressure sensor system, feedback, and support surface can be a standalone system or it can interact with a sensor network. Knowing where pressure is higher than desired allows for a support surface to automatically respond by optimizing the pressure experienced by an MP. If the support surface is unable, by its automatic response means, to correct for the undesired pressure, it can alert a caregiver to decide about providing further care. The data can be used to inform treatment and parameters for care.

Monitoring Mobility/Activity to Determine Need for DVT Prophylaxis

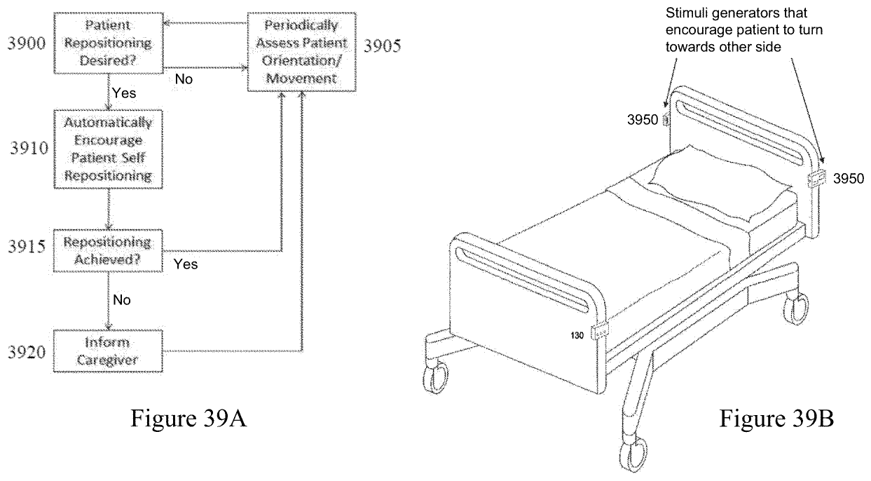

MPs who are immobilized for long periods of time often require prophylaxis to prevent against deep venous thrombosis (DVT). A DVT is a blood clot that forms in a vein (typically in the leg veins) and often is a consequence of venous stasis, which can occur from prolonged immobility. MPs considered at risk for DVTs will generally receive DVT prophylaxis, which can be pharmacologic or mechanical in nature. Pharmacologic DVT prophylaxis consists of systemic anticoagulation (i.e. heparin, enoxaparin) which is delivered to MPs via subcutaneous injections. Mechanical DVT prophylaxis consists of sequential compression devices (SCDs) which are pneumatic compression stockings that are affixed to the legs of MPs and then inflate/deflate in order to promote blood flow and thereby prevent venous stasis. As mentioned previously, a major risk factor for DVTs is prolonged immobility. In an embodiment shown in FIG. 41, the monitoring system is designed to monitor an MP's movements and activity level, and the system can use this information to generate an ‘ activity index’ value for a given MP, shown at steps 4100-4175. The activity index value, shown at 4135, incorporates factors such as: total activity time, amplitude/frequency of movements, acceleration, sustained inactivity time (i.e. how long are the intervals between activity), etc.

Based on the “activity index” score for a particular MP, physicians can decide whether or not DVT prophylaxis is indicated for a particular MP.

To improve the tool, the following factors are incorporated into the analysis, and can be used to generate a DVT “risk score”, shown at 4160:
1. Age, height weight, of MP
2. Is MP a smoker?
3. Does MP have CHF?
4. Is MP on hormonal contraception?
5. Malignancy present?
6. Previous DVT or PE?

Note that weighting for all of the variables can be customized by individual physicians, care providers or institutions, such that they can increase/decrease the threshold for DVT prophylaxis. A set of default values can be initially provided. The system is designed to help physicians objectively decide what treatment is best for their MPs. Currently, physicians have limited objective information to understand how well an MP is ambulating.

Wireless Communication

In certain cases, wireless communication via a device with an antenna can be affected by the surface upon which the antenna lies. For instance, a device on the surface of the skin can have its antenna performance affected by the electromagnetic and dielectric properties of the body. To shield the device from such effects caused by the body, in some embodiments the device may have material between the body and the antenna that shields or reduces the relative effect of the body on the antenna performance. For instance, a material with a high dielectric constant can be placed on the device between the antenna and the body to serve this purpose.

Automatic Decompression Threshold Calculation

In an embodiment, the system can automatically calculate at least one suggested decompression threshold/interval. The decompression threshold/interval refers to the minimum amount of time that an area of the body needs to experience reduced pressure or no pressure in order to adequately re-profuse that area of the body, thereby preventing ischemia and tissue damage. Once an area of the body has surpassed the suggested decompression threshold/interval, that area of the body can once again be pressurized with lower risk for causing tissue damage. The decompression threshold/interval can be calculated by taking into account factors selected from a group comprising: MP characteristics (i.e. Braden score, age, co-morbidities, size/weight/BMI/body mass distribution, etc.), MP variables (mobility, activity, moisture, nutrition level, experienced or estimated sheer force, medical conditions, vital signs, health conditions, health status, previous skin conditions, and medications, etc.), environmental factors (type of bed surface, ambient temperature, humidity, etc.). One or more calculation schemes can be used by the system and selected by the user.

The system can also allow for a decompression threshold that is variable for any given MP. One common usage is to have a decompression threshold for an area of the body vary with the amount of time the area of the body has experienced pressure. For instance, the decompression threshold can take the form of:

D0+D1*[duration of pressure]

where D0 and D1 are constants that can be set or varied or vary automatically based on data about the MP or facility.

The disclosed system, methods, and devices provide an improved method for both calculating the appropriate decompression threshold/interval for a region of the body and also monitoring said region to determine when adequate decompression time has been achieved.

Variables that may affect calculated decompression threshold, include vitals (such as pulse ox, heart rate, breathing rate, blood pressure), time on a given side, duration of pressure orientation, existence of other wounds, MP characteristics (i.e. Braden score, age, co-morbidities, size/weight/BMI/body mass distribution, etc.), MP variables (mobility, activity, moisture, nutrition level, experienced or estimated sheer force, medical conditions, health conditions, health status, previous skin conditions, and medications, etc.), and environmental factors (type of bed surface, ambient temperature, humidity, treatment and prevention techniques used etc.). The system can also accommodate for the ability of different parts of the body may depressurize differently and at different rates.

Indicia on Part Associated with Sensor

An indicia may be associated with an MP sensor so that a user can easily orient the sensor with respect to the MP. The indicia may be a visual indicator, physical feature or shape, or asymmetry. This indicia may be location on the sensor itself, typically the housing or enclosure of the sensor. It can also be located not on the sensor itself, but on something that is in a specific orientation relative to the sensor at some time. One example is a label, stick, adhesive, or element of packaging that can have one or more indicia. These elements may then be separated from the sensor with the user still knowing the orientation of the sensor. Another method is to have a device that can determine the orientation of the sensor, either by mechanical, RF, magnetic, visual, or other communication means.

Flatline Detector

The system can also detect very low to no movement or situations in an MP. Such a situation occurs when the MP's breathing, heartbeat, and other physical motions have stopped. In such a case, the system can very quickly detect such an condition in the MP such that it can note the status and send an alert quickly. In certain cases it would be able to detect the situation in less than a few seconds or in less than one second and alert those who can provide help, possibly within enough time to help the MP. Detection can be much quicker than for systems that detect MP motions suggestive of an abnormal state such as arrhythmias and decompensation. In certain cases there may be ambient movement detected by the system that are not caused by the MP. These movements may easily be disregarded if they fall below the threshold for movements caused by heartbeat or breathing. Alternatively the system may learn what movements are characteristic that don't arise from the MP. Alternatively the system may utilize a separate sensor not on the MP to determine what movements are not arising from the MP and subtract those. Alternatively, the system may use sensors on more than one MP or more than one sensor on the same MP to subtract out the movements that are common, which may be subtracted as those movements arising from outside of the MP. Electrical signal detection from the MP can be used similarly and where movement is described above, electrical signals are replaced in another implementation of the system. Electrical signals and movement detection can be combined as well to further increase the accuracy and robustness of the detection.

### Proning Management Embodiments

Some embodiments of the present invention provide automated or partially automated systems and methods for implementation and management of a proning protocol for a monitored person “MP,” e.g., for treatment of ARDS or other respiratory condition. For example, systems and methods are provided for monitoring and managing the position of an MP, for example a with respect to a proning protocol designed to encourage or “coach” the MP to spend time in a prone position, for example, to improve clinical outcomes for an MP having COVID-19 or other ARDS condition. Some embodiments or situations apply to a conscious, non-intubated MP, while others apply to an intubated or otherwise unconscious MP.

Some embodiments provide a system including a wearable sensor, e.g., sensor 4230 discussed herein, configured to wirelessly communicate with a mobile device (e.g., smartphone or tablet) having a proning user application designed to encourage proning. The wearable sensor may include one or more sensor (e.g., one or more accelerometer, magnetometer, etc.) configured to monitor the MP's orientation over time (e.g., prone, supine, lying on right side, or lying on left side, the MP's torso inclination angle, and/or any other measure of orientation). The mobile device may store or provide an interface to a proning user application (e.g., hosted on the mobile device or on a remote server) designed to manage the MP's position based on sensor data received from the wearable sensor to encourage proning, i.e., to encourage the MP to spend time in the prone position. The proning user application may allow the MP (or caretaker) to select a suitable proning protocol for the MP, which may define timing and/or turning parameters suitable for physiological parameters or other parameters specific to the MP (e.g., sex, age, weight, BMI, disease status, heart rate, respiratory rate, SpO2, etc.). The proning user application may compare sensor data received wirelessly from the wearable sensor to the selected proning protocol and provide feedback to the MP, e.g., via a display screen of the mobile device or via audible or haptic feedback e.g., to instruct or encourage the MP to assume a prone position, or to give encouraging feedback to the MP.

As used herein, “physiological parameters” and “physiological data” include any parameters and data related to the physiology of an MP, including biometric parameters and data (e.g., including parameters and data related to physical positioning and movements of the MP) and any other types of parameters and data related to the MP's bodily functions.

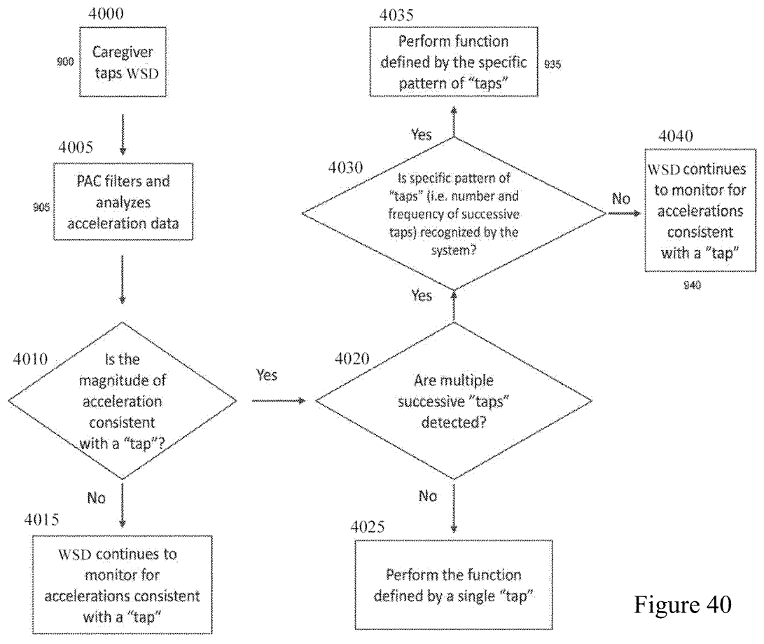

FIG. 42 illustrates an example implementation 4200 of a system for monitoring and managing a proning regimen for an MP, according to one example embodiment. System implementation 4200 includes a wearable sensor 4230 secured to an MP and a user device (e.g., smartphone or other mobile device) 4210 providing a proning user application 4220 confirmed to facilitate the execution of a “proning protocol” that encourages the MP to spend time in a prone position. The wearable sensor 4230 and user device 4210 are configured to wirelessly communicate with each other, e.g., via Bluetooth, RF, RFID, near-field communication (NFC), infrared communications, ZigBee, or other short-range communication protocol (“direct short-range communications”) or via WiFi-based communications.

In some embodiments, the wearable sensor 4230 may comprise or correspond with any of the sensor devices disclosed herein, e.g., sensor 110 shown in FIGS. 1A-1D or sensor 300 shown in FIGS. 3A, 3B, 3F, 12, 13C, 13D, 38A, etc. Such wearable sensor 4230 may be configured to be affixed directly to the MP's 110 skin (e.g., using an adhesive backing provided on the sensor 4230), or may be configured to be secured in a pocket or receptable of an article or clothing (e.g., shirt) or other wearable article (e.g., a chest strap) configured to maintain the sensor 4230 in a substantially fixed position relative to the MP's body.

In other embodiments, the wearable sensor 4230 may comprise a smartwatch or a component of a smartwatch that contains at least one accelerometer and/or magnetometer (e.g., a smartwatch face removable from the watch band). In such embodiments, the sensor 4230 (i.e., smartwatch or smartwatch component) may be configured to be secured in a pocket or receptable of an article or clothing (e.g., shirt) or other wearable article (e.g., a chest strap) configured to maintain the sensor 4230 in a substantially fixed position relative to the MP's body.

Proning user application 4220 may include, for example, a program file or collection or suite of program and data files in any suitable language or format (e.g., JavaScript, JSON, etc.) which may be stored or hosted at the user device 4210 and/or any other device or devices. As used herein, reference to the user device 4210 “providing” a proning user application 4220 means user device 4210 provides a user of the proning user application 4220, or “PUA user” (e.g., the MP, a caregiver, or other person) access to a proning user application 4220, which may include, for example, (a) user device 4210 storing or hosting all or a portion of a proning user application 4220 in local memory, e.g., where the PUA user downloads a proning user application 4220, for example via an iPhone or Android “app store,” or (b) user device 4210 including a web browser that provides the PUA user access to a remotely stored web-based proning user application 4220 (e.g., stored at a back-end web server), e.g., via the internet. In some embodiments in which the user device 4210 accesses a web-based version of the proning user application 4220 via a browser at user device 4210, the browser may download, into cache memory, executable code or other files of the proning user application 4220 sufficient to allow the user device 4210 to locally manage a configuration and/or execution of a proning protocol for the MP, e.g., with little or no required interaction with the web server. In other web-based implementations of proning user application 4220, the browser at user device 4210 may communicate significantly with the web server to manage the configuration and/or execution of a proning protocol for the MP.

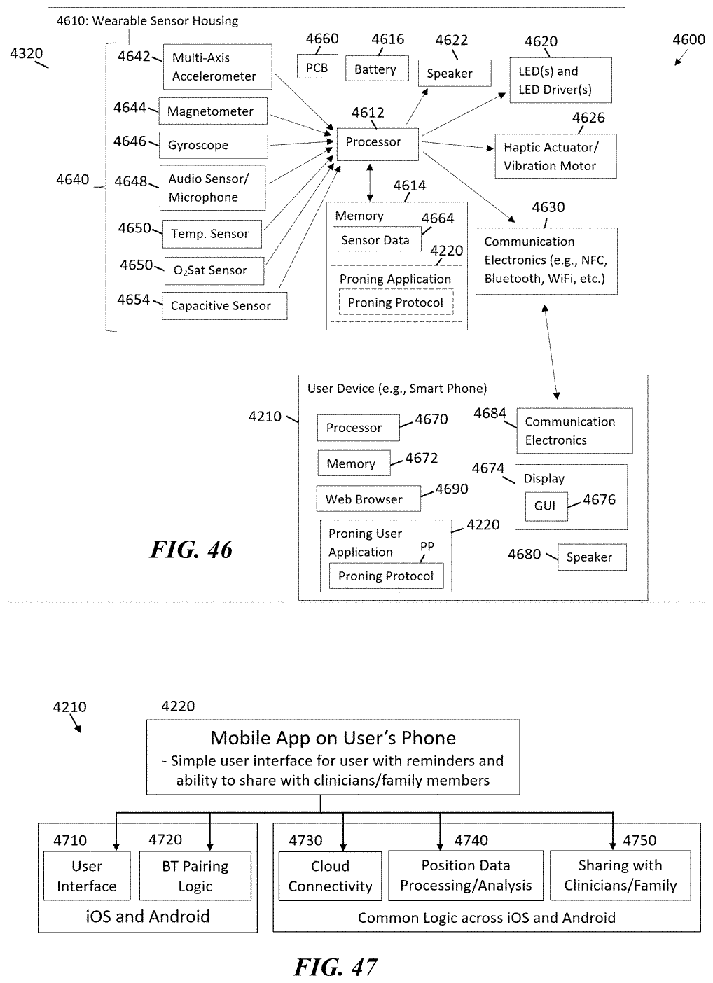

As discussed below with reference to FIG. 46, each user device 4210 may comprise any type of computer device accessible to the PUA user (e.g., a smart phone, smart watch, tablet, laptop, desktop, assistant device (Echo, etc.), or any other type of computer), and may include at least one processor, at least one memory device, and a plurality of user device input/output devices (“I/O components”), including for example a display device (e.g., LED touchscreen) that provides a graphical user interface (GUI) to the PUA user, a speaker, a microphone, and a camera. The proning user application 4220 may engage with the PUA user via one or more of such user device I/O components. For example, proning user application 4220 may display information to and receive input from the PUA user via the GUI, and output notifications (e.g., audible repositioning notifications) via the speaker.

In any embodiment disclosed herein, the wearable sensor 4230 (or each wearable sensor 4230 in the case of multiple wearable sensors 4230 secured to an MP) may be secured to the MP or in an article worn by the respective MP at any suitable location on the MP's body, for example, (a) on the MP's abdomen, e.g., just above the MP's navel, (b) on the MP's chest, e.g., over the sternum or over a pectoral muscle, (c) on the MP's hip or pelvis, or (d) on the MP's back, e.g., between the shoulder blades or over one of the lungs (e.g., near the lower base of the lung). As discussed above, e.g., with respect to FIG. 9, the wearable sensor 4230 may include visible indicia or may incorporate a sensor-based system for facilitating or ensuring a proper location and/or orientation of the wearable sensor 4230 relative to the MP's body, e.g., to provide effective functionality of the wearable sensor 4230.

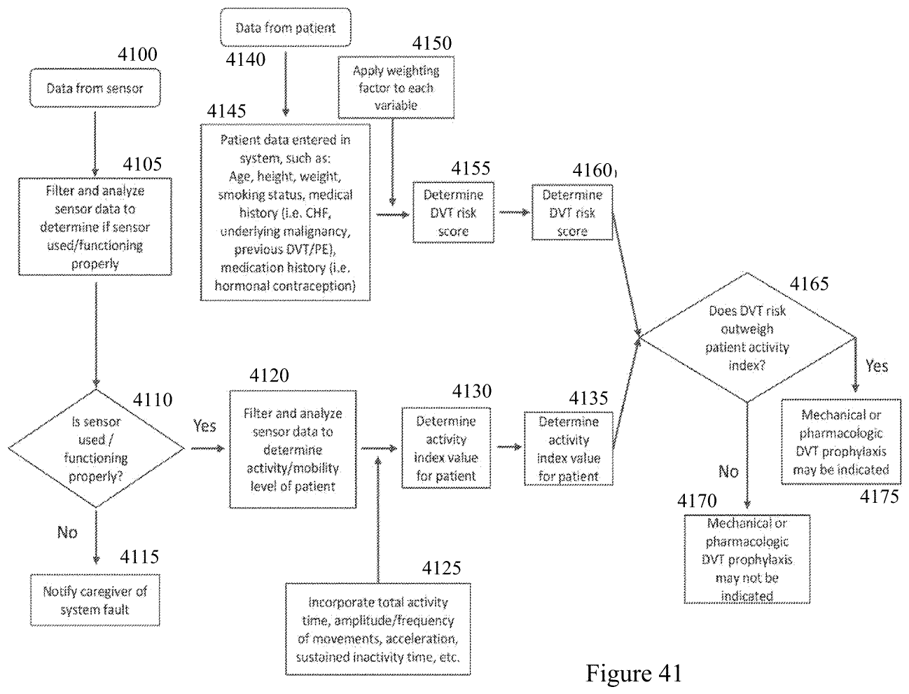

FIG. 43 illustrates another example implementation 4300 of a system for monitoring and managing a proning regimen for one or more MPs (e.g., with each MP executing a respective proning protocol), in which the MPs may be monitored by a caretaker “CT,” according to one example embodiment. System implementation 4300 may represent an example implementation in a hospital, medical facility, nursing home, or other environment that provides managed care. For example, each MP may be located in a different room or on a different bed within a common medical or care facility.

As shown, the example implementation 4300 may involve multiple MP's, indicated at MP_{A} and MP_{B}, each having a respective wearable sensor 4230_{A} and 4230_{B} and respective user device 4210_{A} and 4210_{B}. Each user device 4210_{A} and 4210_{B} may provide a respective instance of the proning user application 4220, as indicated at 4220_{A} and 4220_{B}. Each proning user application instance (e.g., 4220_{A} and 4220_{B}) may have customized settings and/or stored data specific to the MP corresponding with that proning user application instance. Data associated with each proning user application instance (e.g., 4220_{A} and 4220_{B}) may be stored locally at each respective user device 4210, or data for multiple proning user application instances may be stored collectively at a back-end web server. It should be understood that although two MPs are shown, the example implementation 4300 may involve any other number of MPs, user devices 4210, and proning user application instances 4220.

Each wearable sensor 4230_{A}, 4230_{B} may be configured to communicate with its corresponding user device 4210_{A}, 4210_{B}, e.g., by Bluetooth or other direct short-range communications, e.g., as discussed above regarding FIG. 42. In addition, each wearable sensor 4230_{A}, 4230_{B} and/or each user device 4210_{A}, 4210_{B} may communicate with one or more caretaker devices (“CT devices”) 4310 via any suitable wireless or wired communication protocol(s), to provide one or more CTs an interface to monitor and/or manage the proning regimen implemented for each respective MP. A caretaker (CT) may be any medical professional or other person (e.g., family member of MP) that uses a CT device 4310 to monitor the proning regiment at least one MP.

In the illustrated example, each wearable sensor 4230_{A}, 4230_{B} and each user device 4210_{A}, 4210_{B} is configured to communicate with CT device(s) 4310 via WiFi communications, e.g., via at least one WiFi access point or router 4320. Each CT device 4310 may be connected to the WiFi access point or router 4320 by wireless WiFi connection or by wired connection (e.g., Ethernet). In other implementations, wearable sensors 4230_{A}, 4230_{B} are configured to communicate with user devices 4210_{A}, 4210_{B} (e.g., via Bluetooth or other direct short-range communications), and user devices 4210_{A}, 4210_{B} (but not wearable sensors 4230_{A}, 4230_{B}) are configured to communicate with CT device(s) 4310 via WiFi-based communications. In still other implementations, wearable sensors 4230_{A}, 4230_{B} are configured communicate with user devices 4210_{A}, 4210_{B} (e.g., via Bluetooth or other direct short-range communications), and wearable sensors 4230_{A}, 4230_{B} (but not user devices 4210_{A}, 4210_{B}) are also configured communicate with CT device(s) 4310 via WiFi-based communications.

Each CT device 4310 may comprise any type of computer device accessible to a CT, e.g., a laptop, desktop, smartphone, or any other type of mobile or stationary computer device, and may include at least one processor, at least one memory device, and a plurality of CT device input/output devices (“I/O components”), including a display device (e.g., LED monitor or touchscreen) that provides a graphical user interface (GUI) to a CT and/or any other suitable I/O components. Each CT device 4310 may provide a proning management application 4330 with which the CT may engage with the CT via the GUI and/or other I/O components of the CT device 4310.

The proning management application 4330 provided at a CT device 4310 may interface with one or more proning user application instances 4220 and provide different or additional monitoring and/or management functionality as compared with the proning user application 4220. For example, the proning management application 4330 may provide the CT an interface to each proning user application instance 4220_{A} and 4220_{B} and/or each wearable sensors 4230_{A}, 4230_{B} to configure a proning protocol for each respective user MP_{A} and MP_{B}. Further, the proning management application 4330 may receive 1\H-specific data from each proning user application instance 4220_{A}, 4220_{B} and/or from each wearable sensor 4230_{A}, 4230_{B} during the execution of each proning protocol, and provide the CT an interface to monitor and/or manage the proning protocol execution by each respective user MP_{A} and MP_{B}. For example, the proning management application 4330 may simultaneously display proning protocol execution data for each respective user MP_{A} and MP_{B}, or allow the CT to selectively access and view the proning protocol execution data for a selected user MP_{A} or MP_{B}. The proning management application 4330 may display or otherwise output any suitable alarms or other notifications related to the proning protocol execution by each user MP_{A} and MP_{B}. In some embodiments, the proning management application 4330 may also allow the CT to remotely initiate an alarm or other notification to be output by a selected user devices 4210_{A}, 4210_{B} or wearable sensors 4230_{A}, 4230_{B}, e.g., an audible alert output by a user device 4210 or an audible or haptic alert output by a wearable sensor 4230.

Proning management application 4330 may include, for example, a program file or collection or suite of program and data files in any suitable language or format (e.g., JavaScript, JSON, etc.) which may be stored or hosted at a CT device 4310 and/or any other device or devices. As used herein, reference to the CT device 4310 “providing” a proning management application 4330 means CT device 4310 provides a CT access to a proning management application 4330, which may include, for example, (a) CT device 4310 storing or hosting all or a portion of a proning management application 4330 in local memory, e.g., where the CT or other user downloads a proning management application 4330, or (b) CT device 4310 including a web browser that provides the CT access to a remotely stored web-based proning management application 4330 (e.g., stored at a back-end web server), e.g., via the internet. In some embodiments in which the CT device 4310 accesses a web-based version of the proning management application 4330 via a browser at CT device 4310, the browser may download, into cache memory, executable code or other files of the proning management application 4330 sufficient to allow the CT device 4310 to locally manage a configuration and/or execution of a proning protocol for each MP, e.g., with little or no required interaction with the web server. In other web-based implementations of proning management application 4330, the browser at CT device 4310 may communicate significantly with the web server to manage the configuration and/or execution of a proning protocol for each MP.

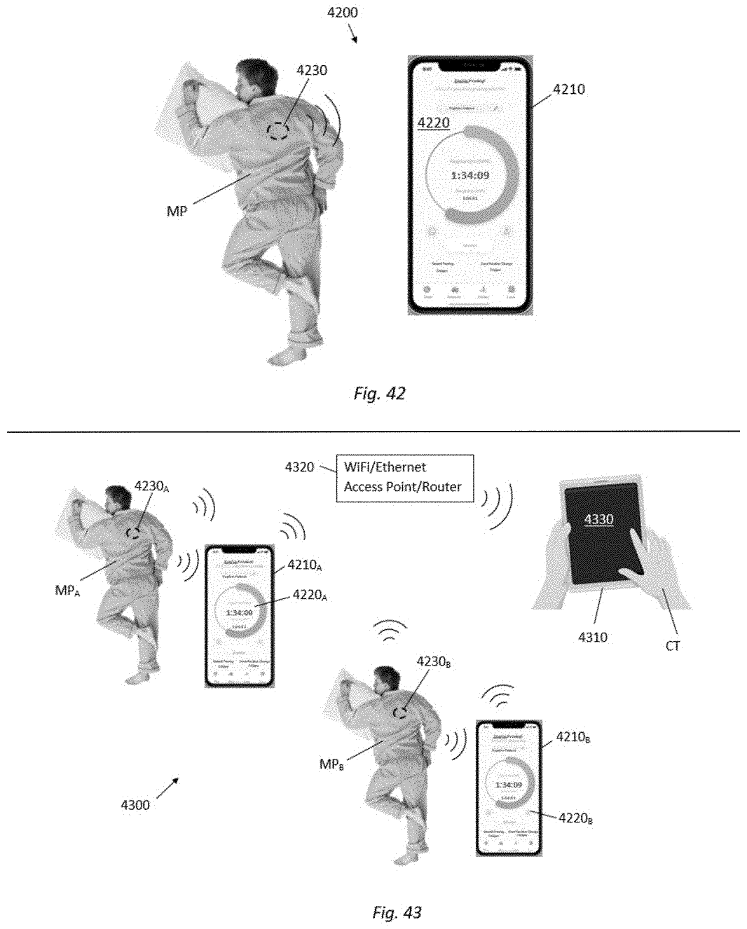

FIG. 44 illustrates another example implementation 4400 of a system for monitoring and managing a proning regimen for one or more MPs (e.g., with each MP executing a respective proning protocol), in which the MPs may be remotely monitored by a caretaker CT, according to one example embodiment. System implementation 4400 may represent, for example, an implementation in which home-bound MPs 4230 are monitored by a remote CT, e.g., a doctor, nurse, or other caregiver at a hospital, medical office, or anywhere else.

As shown, the example implementation 4400 may involve multiple MP's, indicated at MP_{A} and MP_{B}, remotely monitored via the internet (or other wide-area network) 4410. Each wearable sensor 4230_{A}, 4230_{B} and/or each user device 4210_{A}, 4210_{B} may be configured to communicate with CT device(s) 4310 via the internet 4410 and/or any additional communications devices, e.g., WiFi access points or routers 4320. Each CT device 4310 may be connected to an access point or router 4320 by wireless WiFi connection or by wired connection (e.g., Ethernet).

In embodiments disclosed herein, the proning user application 4220 provided at a user device 4210 may be configured to facilitate a configuration of a proning protocol for a particular MP based on any suitable proning protocol configuration input data, which may include (a) user input from the MP or other PUA user via the proning user application 4220 at the user device 4210, (b) user input from a CT via a proning management application 4330 provided at a CT device 4310, and/or (c) sensor data collected by a wearable sensor device 4230 and/or other sensor(s) associated with the MP. Example types of proning protocol configuration input data that may be entered by a user, e.g., via the proning user application 4220 or via a proning management application 4330, include:
(a) a selection of a defined proning protocol, e.g., Hopkins Protocol, Vanderbilt Protocol, Boston Protocol, etc.;
(b) the age of the MP;
(c) the sex of the MP;
(d) the weight, height, body mass index (BMI), or other size or mass-related parameters;
(e) the blood type of the MP;
(f) demographic information regarding the MP;
(g) lab test results, e.g., CBS, chemistry, etc.;
(h) medical imaging results, e.g., CT, X-ray imaging;
(i) pulmonary function tests, e.g., spirometry, plethysmography, etc.;
(j) past medical history of the MP, e.g., diseases, other medical conditions, operations or medical procedures, medical history of family members, etc.;
(k) history of pressure injuries;
(l) list of co-morbidities;
(m) lab test results or medical diagnosis for one or more particular disease or medical condition, e.g., SARS-CoV-2 infection (COVID-19);
(n) the presence or absence of predefined symptoms associated with a particular disease or medical condition, e.g., SARS-CoV-2 infection (COVID-19);
(o) severity of infection, disease or medical condition (e.g., SARS-CoV-2 infection (COVID-19);
(p) history of infection, e.g., history of SARS-CoV-2 infection (COVID-19) as evidenced by antibody test results;
(q) type and/or features of the MP's bed or other support surface;
(r) clinical setting, e.g., home, hospital, etc.;
(s) time of day;
(t) sleeping patterns of the MP; and/or
(u) any other type of data that may be input by a user.

Example types of proning protocol configuration input data that may be automatically obtained by a wearable sensor 4230 or other sensor(s) interacting with the MP, e.g., for a defined protocol configuration period during which the MP is analyzed for creating a customized or MP-specific proning protocol, may include:
(a) heart rate data (e.g., determined based on acceleration data generated by at least one accelerometer or audio data generated by an audio sensor, e.g., integrated in the wearable sensor 4230 or a separate sensor);
(b) respiratory rate data (e.g., determined based on acceleration data generated by at least one accelerometer or audio data generated by an audio sensor, e.g., integrated in the wearable sensor 4230 or a separate sensor);
(c) body temperature data (determined by a temperature sensor, e.g., integrated in the wearable sensor 4230 or a separate sensor);
(d) SpO2 and/or SaO2 data (e.g., generated by a fingertip pulse oximeter or an oximeter sensor integrated in the wearable sensor 4230);
(e) coughing, wheezing, or other respiratory sound data (determined based on acceleration data generated by at least one accelerometer or audio data generated by an audio sensor, e.g., integrated in the wearable sensor 4230 or a separate sensor);
(f) lung-specific data (e.g., determined based on an audio sensor (in wearable sensor 4230 or otherwise) located proximate a selected lung, or based on a pair of audio sensors (in a pair of wearable sensors 4230 or otherwise) each located proximate a respective lung);
(g) blood gas analysis data (e.g., arterial oxygen content, pH, etc.);
(h) MP body position and activity information (e.g., monitored orientation data, upright angle data, time out of bed, ambulation data, e.g., steps, velocity, etc.), e.g., as monitored by a wearable sensor 4230 prior to execution of the proning protocol; and/or
(i) any other type of data that may be collected by sensor(s) provided in the wearable sensor 4230 and/or provided by separate sensor(s) interacting with the MP.

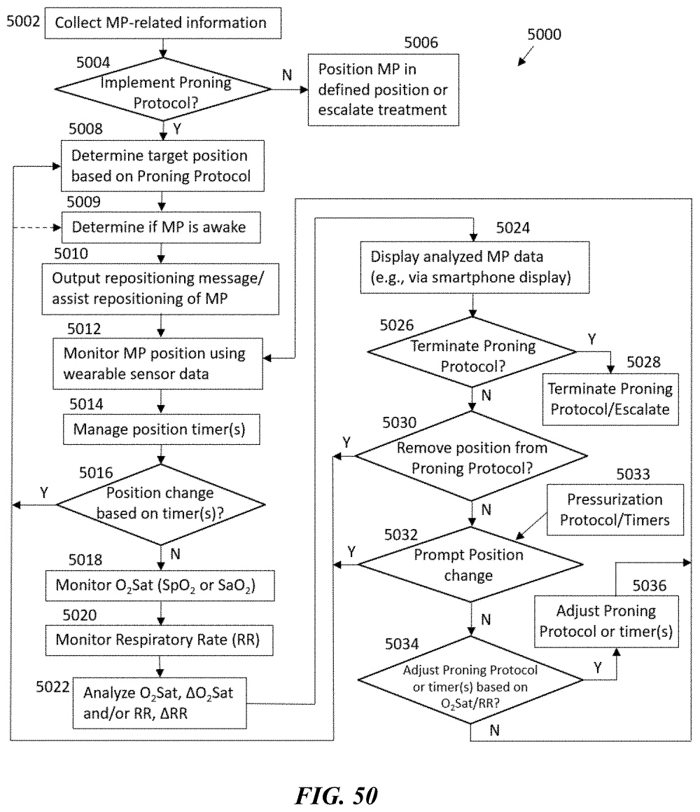

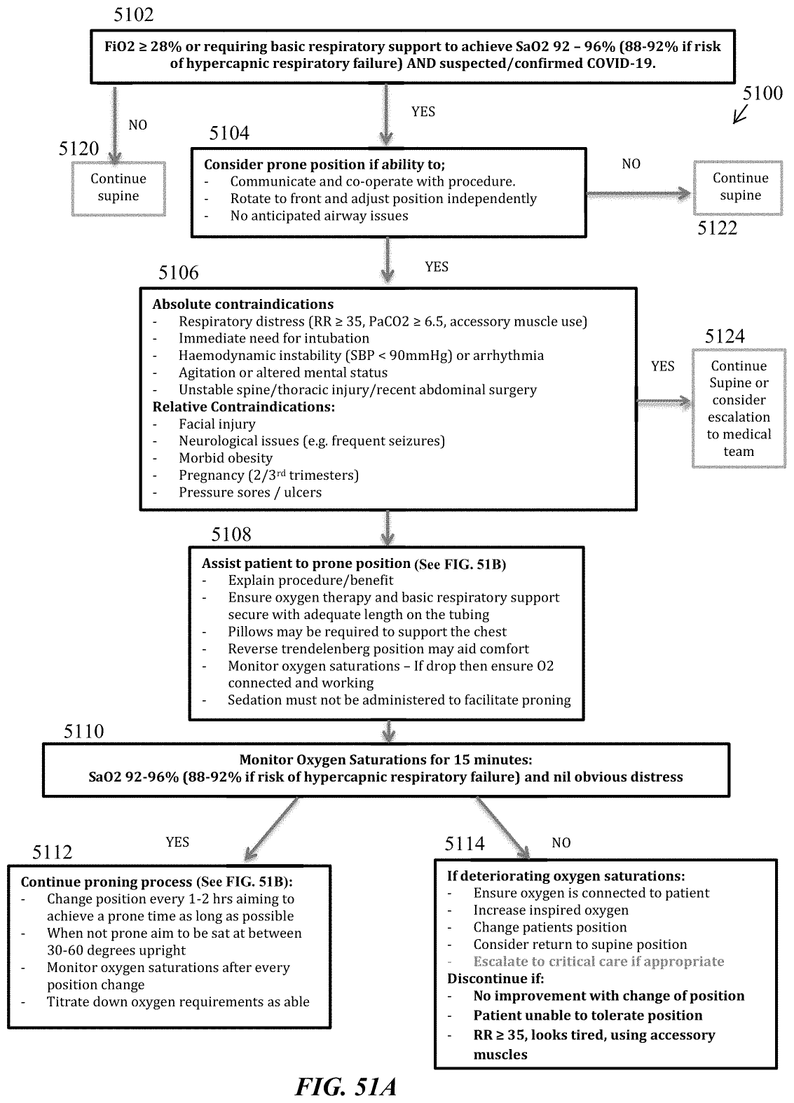

The proning user application 4220 may utilize any of such proning protocol configuration inputs or any other suitable inputs for selecting or defining protocol parameters for a customized proning protocol for the particular MP. Example proning protocol parameters include:
(a) a defined set of body positions involved in the proning protocol; e.g., prone and non-prone; or prone, lying on left side, lying on right side, and sitting with elevated torso angle; or any other defined set of body positions;
(b) parameters defining each body position involved in the proning protocol, e.g., angular thresholds regarding rotational orientations relative to one or more axes (e.g., three orthogonal axes) that define positional limits of each body position, including at least angular thresholds relative to one or more axes that define a prone position;
(c) respective maximum and/or minimum durations (including continuous and/or cumulative durations) to be spent by the MP in each body position involved in the proning protocol, including the prone position and/or other body positions(s) (e.g., 30 min minimum and 120 min maximum duration for each period spent in the prone position);
(d) threshold values for sensor-based data (e.g., sensor values and/or rates of change of sensor values) regarding one or more physiological parameters of the MP that automatically trigger one or more actions, e.g., any of the actions set forth in example method 4900 (FIG. 49) or method 5000 (FIG. 50), discussed below (e.g., terminating the proning protocol, removing a particular body position from the proning protocol, triggering a repositioning notification, or adjusting other proning protocol parameter(s));
(e) a rate of increase and/or rate of decrease for a prone time counter representing time spent in a prone position (e.g., an accumulated prone time counter representing a cumulative time spent in a prone position);
(f) a rate of increase and/or rate of decrease for a non-prone time counter representing time spent out of a prone position (e.g., an accumulated non-prone time counter representing a cumulative time spent out of a prone position);
(g) algorithms or mathematical relationships between two or more measured factors or proning protocol parameters, for example (i) a mathematical relationship defining an automated adjustment of one or more first parameters (e.g., minimum duration to be spent in a particular position) as a function of one or more second parameters (e.g., sensor-detected SaO2, calculated rate of change of SaO2, sensor-detected respiratory rate, calculated rate of change of respiratory rate, heart rate, respiratory rate, body temperature, or coughing frequency), or (ii) a mathematical relationship defining a rate of increase and/or rate of decrease for a prone time counter (e.g., an accumulated time counter) as a function of real-time sensor-detected heart rate, respiratory rate (breathing rate), body temperature, or coughing frequency); and/or
(h) any other rules or parameters relevant to generation and/or implementation of a proning protocol for the relevant MP.

After facilitating the configuration of the proning protocol for the particular MP (e.g., by selecting or defining any of the example proning protocol rules or parameters listed above based on any of the various proning protocol configuration inputs listed above), the proning user application 4220 may facilitate an execution of the proning protocol for the MP. To facilitate the proning protocol execution, the proning user application 4220 may (a) collect sensor data from a wearable sensor 4230 (or multiple wearable sensors 4230) secured to the MP and/or one or more other sensors arranged to interact with the MP (e.g., a fingertip pulse oximeter, an EKG monitor, a breathing monitor, etc.); and (b) compare or analyze the collected sensor data with respect to particular rules or parameters of the proning protocol to analyze the MP's performance over time. The proning user application 4220 may generate various outputs based on such analysis, including information displayed via the user device GUI and/or alerts or notifications for output to the MP, e.g., via audible alerts output by a speaker of the user device 4210 or haptic feedback output by the wearable sensor 4230 (upon instruction communicated from the user device 4210 to the wearable sensor 4230). Such outputs may indicate, for example, instructions or other prompts for a position change of the MP along with a body position the MP should turn or be turned to, instructions or other prompts for the MP should get out of bed, instructions or other prompts for the MP to sit up, instructions or other prompts for the MP to ambulate and details regarding such ambulation (e.g., time, distance, etc.), etc.

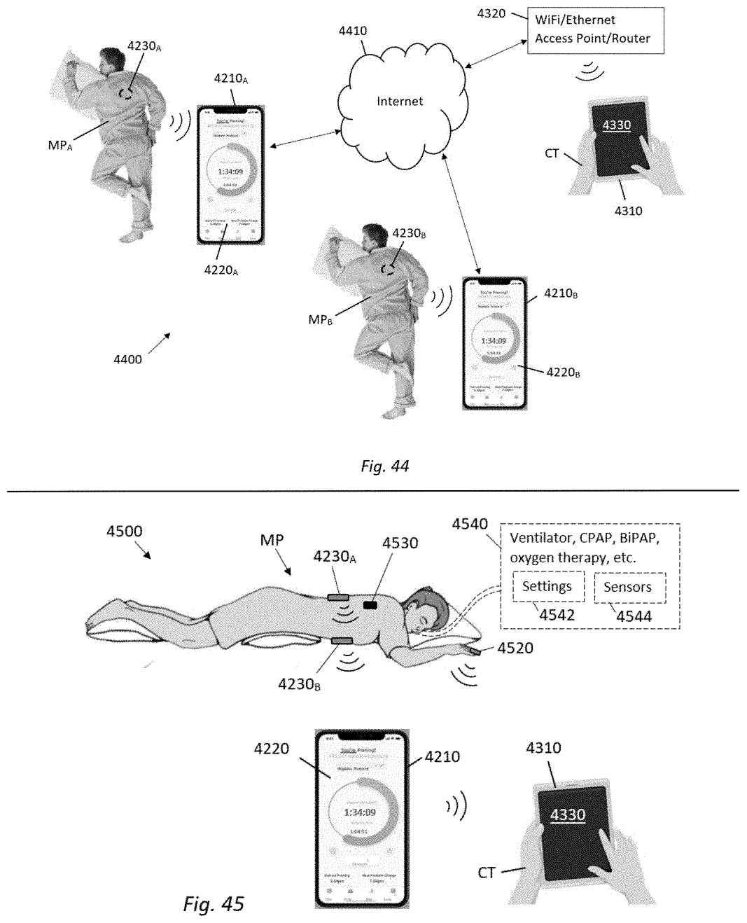

Example types of sensor data collected during execution of the proning protocol, and analyzed with respect to the proning protocol rules or parameters, include:
(a) MP orientation data representing the orientation of the MP with respect to one or more axes (e.g., as determined based on acceleration data generated by at least one accelerometer and/or magnetization data generated by at least one magnetometer, e.g., integrated in the wearable sensor 4230);
(b) MP ambulation or physical activity data (e.g., as determined based on acceleration data generated by at least one accelerometer, magnetization data generated by at least one magnetometer, e.g., integrated in the wearable sensor 4230, or based on GPS data generated by a GPS system provided in the user device 4210 or in the wearable sensor 4230);
(c) heart rate data (e.g., determined based on acceleration data generated by at least one accelerometer or audio data generated by an audio sensor, e.g., integrated in the wearable sensor 4230 or a separate sensor);
(d) respiratory rate data (e.g., determined based on acceleration data generated by at least one accelerometer or audio data generated by an audio sensor, e.g., integrated in the wearable sensor 4230 or a separate sensor);
(e) body temperature data (determined by a temperature sensor, e.g., integrated in the wearable sensor 4230 or a separate sensor);
(f) SpO2 and/or SaO2 data (e.g., generated by a fingertip pulse oximeter 4520, e.g., as shown in FIG. 45, or an oxygen station sensor 4650 integrated in the wearable sensor 4230);
(g) coughing, wheezing, or other respiratory sound data (determined based on acceleration data generated by at least one accelerometer or audio data generated by an audio sensor, e.g., integrated in the wearable sensor 4230 or a separate sensor);
(h) lung-specific data (e.g., determined based on an audio sensor (in wearable sensor 4230 or otherwise) located proximate a selected lung, or based on a pair of audio sensors (in a pair of wearable sensors 4230 or otherwise) each located proximate a respective lung);
(i) for an MP that is intubated and assisted by a ventilator, or connected to a CPAP machine, BiPAP machine, or other oxygen therapy machine or device for MP supplemental oxygen and/or respiratory assistance, the proning user application 4220 receive from such respiratory assistance device (a) one or more stored device settings regarding oxygen and/or respiratory assistance provided to the MP (e.g., FiO₂, positive end-expiratory pressure (PEEP), inspiratory airflow, tidal volume (VT), respiratory rate, oxygen flow rate, etc.), and/or sensor data generated by integrated sensor(s) of such respiratory assistance device (e.g., respiratory rate, respiratory effort, expired oxygen and/or CO₂, heart rate, etc.); and/or
(j) any other type of data that may be collected by sensor(s) provided in the wearable sensor 4230 and/or provided by separate sensor(s) interacting with the MP.

FIG. 45 illustrates an example implementation 4500 in which multiple example sensors are secured to an MP, including any combination of the following: (a) a first wearable sensor 4230_{A} secured at the MP's back (e.g., to detect MP orientation data and lung-related audio data), (b) a second wearable sensor 4230_{B} secured at the MP's chest or abdomen (e.g., to detect MP orientation data and heart-related audio data), (c) a fingertip pulse oximeter 4520 to detect SpO2 data. Each sensor 4230_{A}, 4230_{B}, and 4520 may be configured to wirelessly communicate data to a user device 4210 providing a proning user application 4220, e.g., continuously in real-time, at defined intervals (e.g., every 10 seconds), or upon certain defined events (e.g., a detected change in physical orientation or other measured physiological parameter). The proning user application 4220 may be able to analyze the data received from sensors 4230_{A}, 4230_{B}, and 4520 with respect to the rules or parameters of a particular proning protocol configured for the MP, and output relevant data via the user device GUI and/or via audible and/or haptic notifications. For example, proning user application 4220 may apply an algorithm (defined by a set of rules for the proning protocol) to (a) orientation data received from first wearable sensor 4230_{A} (b) respiratory rate data received from first wearable sensor 4230_{B}, and (c) SpO2 data received from pulse oximeter 4520 to (i) monitor progress with the proning protocol and (ii) identity alert or notification conditions that trigger the output of an appropriate alert or notification.

In some embodiments, the user device 4210 or proning user application 4220 may be configured to cooperate with a proning management application 4330 provided at a CT device 4310, to allow CT monitoring and/or management of the MP's proning regimen, e.g., as discussed above regarding FIG. 43.

In some embodiments, additional sensor(s) and/or medical devices may be secured to the MP or otherwise arranged to monitor one or more physiological parameters of the MP, which may be provided in addition to one or more of sensors 4230_{A}, 4230_{B}, and 4520 discussed above, to provide additional feedback for regarding the MP for use by proning user application 4220 and/or proning management application 4330. As a first example, in some embodiments, the MP may be connected to a ventilator (i.e., the MP may be intubated and unconscious), CPAP machine, BiPAP machine, or other oxygen therapy machine or device for providing the MP supplemental oxygen and/or respiratory assistance. As shown, such device 4540 may store a number of device settings 4542 regarding oxygen and/or respiratory assistance provided to the MP (e.g., FiO₂, positive end-expiratory pressure (PEEP), inspiratory airflow, tidal volume (VT), respiratory rate, oxygen flow rate, etc.). Further, such device 4540 may include one or more sensors 4544 configured to measure or monitor one or more physiological parameters of the MP (e.g., respiratory rate, respiratory effort, expired oxygen and/or CO₂, heart rate, etc.).

As a second example, in some embodiments, system 4500 may include one or more additional sensors 4530 secured to or otherwise arranged to interface with the MP, for example, an EKG or other heart monitoring device, an electroencephalogram (EEG) sensor, acoustic sensor(s), etc.

FIG. 46 illustrates an example arrangement 4600 of a wearable sensor 4320 configured to communicate with a user device (e.g., smart phone) 4210 for monitoring and managing a proning regiment for an MP to which the wearable sensor 4320 is affixed. In some embodiments, wearable sensor 4320 represents an implementation of wearable sensor 110 or wearable sensor 300 shown in FIGS. 1A-1D, 3A-3B, 3F, 12, 13C, 38A, etc., discussed above.

The wearable sensor 4320 may include any suitable hardware, firmware, and/or software components provided in a sealed housing 4610. In some embodiments, the wearable sensor housing 4320 may have a small form factor with a low profile, and may be flexible to conform to the MP's body. The wearable sensor housing 4320 may be provided with a removable adhesive for securing the housing 4320 directly to the MP's body or to an article or dressing worn by the MP.

As shown, the example wearable sensor 4320 may include a processor 4612, memory 4614, a battery 4616, LEDs and LED drivers 4620, a speaker 4622, a haptic actuator 4626, communication electronics 4630, and a group of sensors 4640. In this example, the sensors 4640 include a multi-axis accelerometer 4642, a multi-axis magnetometer 4644, a gyroscope 4646, an audio sensor or microphone 4648, a temperature sensor 4650, an oxygen saturation sensor 4652, and a capacitive sensor 4654. In other embodiments, wearable sensor 4320 may include any other types of sensors, or may omit any of the sensors shown in FIG. 46. Any of the various components discussed above may be mounted to a PCB 4660, e.g., a flexible PCB providing further flexibility to wearable sensor 4320. For example, PCB 4660 may comprise a Fury PCB (Rev 2), or a Sentinel board with NFC added.

Processor 4612 and memory 4614 may comprise any suitable processor and memory devices for processing and storing data, for example corresponding with processor 125 or 310 shown in FIG. 1B, 3A, 3B, or 3F and memory 135 shown in FIG. 1B. Battery 4616 may comprise a rechargeable or non-rechargeable battery, a CR2032 or other suitable coin-cell battery, for providing power to the various electronics of wearable sensor 4320.

Memory 4614 may store any data and/or code for implementing the functions of wearable sensor 4320. In the illustrated example, memory 4614 may store sensor data 4664 generated by sensors 4640, e.g., temporarily cached for period forwarding to user device 4210, or stored for a longer period.

In some embodiments, memory 4614 may also store a proning user application 4220, which may be executed by processor 4612. Thus, in some embodiments, wearable sensor 4320 may provide a self-contained system for implementing a proning protocol for the MP, without requiring assistance from a user device 4210 and/or CT device 4310. In some embodiments, the wearable sensor 4320 may be preloaded with a proning protocol, or a set of proning protocols from which a particular protocol may be selected for the particular MP via user interaction with the wearable sensor 4320 itself, e.g., by pressing a button on the wearable sensor 4320 to cycle through the different preloaded proning protocols, wherein the currently selected proning protocol may be indicated by selective illumination of LED(s) 4620 or by audible output via speaker 4622. In other embodiments, a user may create a customized proning protocol for the particular MP by interaction with a proning user application 4220 at a user device 4210 (e.g., to input MP-specific data and/or select particular protocol rules or parameters) and/or interaction with a proning management application 4330 at a CT device 4310. The customized proning protocol may be stored in memory 4614 of the wearable sensor 4320 and executed by processor 4612, without requiring assistance from a user device 4210 and/or CT device 4310, such that the wearable sensor 4320 provides a self-contained execution of the customized proning protocol.

LED(s) and LED driver(s) 4620 may include one or multiple LEDs and associated drivers arranged in any suitable manner, for example according to any of the example embodiments discussed and illustrated herein. Haptic actuator 4626 may comprise a vibration motor or other device configured to provide haptic feedback to the MP to which the wearable sensor is secured, e.g., to communicate alerts or other notifications to the MP. The haptic actuator 4626 may be configured to output different haptic signals (e.g., providing vibrations with different magnitudes, frequencies, vibration patterns, etc.) to communicate different messages to the MP, e.g., different types of repositioning notifications.

Communication electronics 4630 may include any electronics, e.g., transmitters, receivers, transceivers, antennas, A/D circuits, or other electronics configured to transmit and/or receive wireless communications according to (a) one or more direct short-range communications (e.g., Bluetooth, RF, RFID, near-field communication (NFC), infrared communications, ZigBee, etc.), (b) WiFi communications, and/or (c) GPS communications.

Like wearable sensor 4320, the user device 4210 may include any suitable hardware, firmware, and/or software components suitable to provide the functionality of user device 4210 disclosed herein, in particular to cooperate with wearable sensor 4320 to configure and manage the execution of a proning protocol for an MP. As discussed above, user device 4210 may comprise a smart phone, smart watch, tablet, laptop, desktop, assistant device (Echo, etc.), or any other type of computer device suitable to provide a proning user application 4220 to a PUA user. In the illustrated example the user device 4210 includes (among other components) a processor 4670, memory 4672, a web browser 4690, a display device 4674 (e.g., touchscreen or monitor) configured to display a GUI 4676, a speaker 4680, and communication electronics 4686.

As discussed above, the proning user application 4220 may be (a) downloaded and stored at the user device 4210, e.g., in memory 4672, or (b) hosted remotely and accessible via web browser 4690, wherein portions of the application code may be stored locally in the web browser cache for local execution by processor 4670.

Communication electronics 4684 may include any electronics, e.g., transmitters, receivers, transceivers, antennas, A/D circuits, or other electronics configured to transmit and/or receive wireless communications according to (a) one or more direct short-range communications (e.g., Bluetooth, RF, RFID, near-field communication (NFC), infrared communications, ZigBee, etc.), (b) WiFi communications, and/or (c) GPS communications.

In some embodiments, a user may create a customized proning protocol “PP” for a particular MP by interaction with proning user application 4220 at GUI 4676, e.g., to input MP-specific data and/or select particular protocol rules or parameters. As discussed above, the configuration of the proning protocol PP for the MP may utilize relevant sensor data regarding the MP, e.g., sensor data collected by one or more sensors 4640 of wearable sensor device 4320. Thus, wearable sensor device 4320 may communicate sensor data from sensor(s) 4640 to user device 4210 for use by proning user application 4220 as input for configuring the proning protocol PP for the MP. The customized proning protocol generated by the proning user application 4220 may be stored in memory 4672 of the user device 4210, and in some embodiments communicated to wearable sensor 4320, e.g., for storage in memory 4614.

During execution of the proning protocol PP, wearable sensor device 4320 may communicate sensor data generated by sensor(s) 4640 to user device 4210 (e.g., in real-time or by periodically forwarding sensor data cached in memory 4614) for use by proning user application 4220, to analyze the execution of the proning protocol PP. For example, proning user application 4220 may compare sensor data received from the wearable sensor 4320 to proning protocol rules or parameters of the proning protocol PP to monitor the MP's progress with respect to the proning protocol PP, and to generate alerts or other notifications based on such analysis. For example, the proning user application 4220 may dynamically adjust accumulated time counters for one or more orientations of the MP, e.g., a prone position and at least one non-prone position. The proning user application 4220 may determine when an accumulated time counter for a respective orientation (e.g., a prone position) exceeds a defined threshold time, and in response, generate a repositioning notification and cause such notification to be output by the user device 4210 and/or the wearable sensor 4320, to thereby instruct or encourage a repositioning of the MP to or away from a particular orientation, e.g., a prone position. The proning user application 4220 may also generate various graphical or other visual displays of information for output via GUI 4676, for example (a) the current time counter value for one or more respective orientations, (b) other sensor data collected by wearable sensor 4320 and/or other sensors interacting with the MP, (c) historical data regarding the MP's performance during the execution of the proning protocol PP, (d) alarms or notifications, and/or any other types of information.

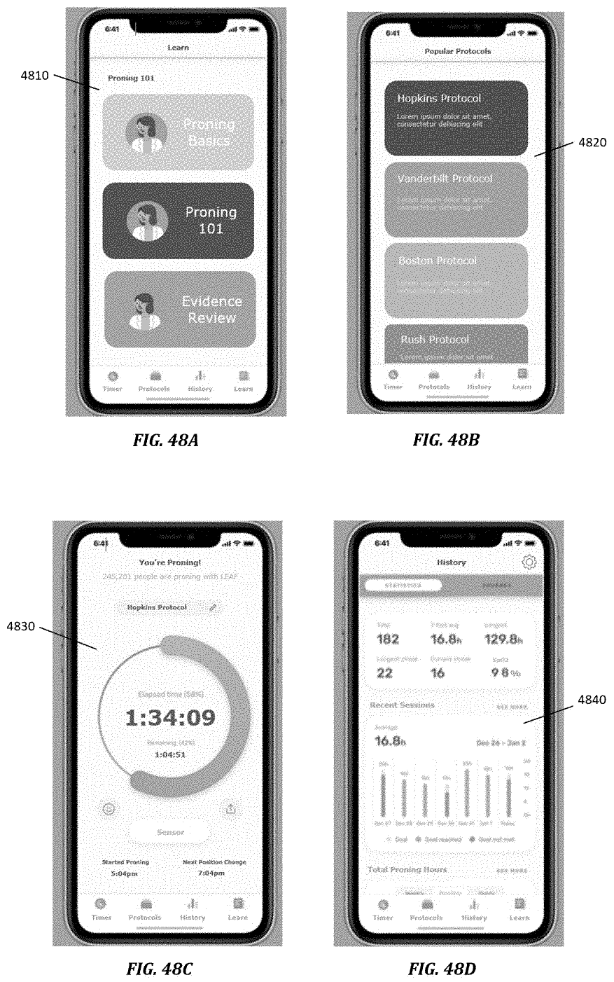

FIG. 47 illustrates selected components of an example user device 4210 (e.g., a smart phone or other computer device) running an iOS or Android operating system, according to example embodiments. As shown, a proning user application 4220 may be provided by the iOS or Android based device 4210, e.g., where the application 4220 is hosted locally, e.g., downloaded to memory of the device 4210 or where access is hosted remotely (e.g., at one or more web sever) and accessible via a web browser provided at the device 4210. As shown, the user device 4210 may include an iOS or Android based or compatible user interface 4710 and Bluetooth paring logic 4720. In addition, user deice 4210 may include any logic (e.g., software and/or firmware) for providing the various functionality associated with a proning management system, e.g., cloud connectively logic 4730, logic for processing or analyzing MP position/orientation data 4740, and communication logic 4750 for sharing data or otherwise communicating with other devices, e.g., CT device(s) 4310 providing an instance of proning management application 4330 and/or other user device(s) 4210 providing an instance of proning user application 4220.

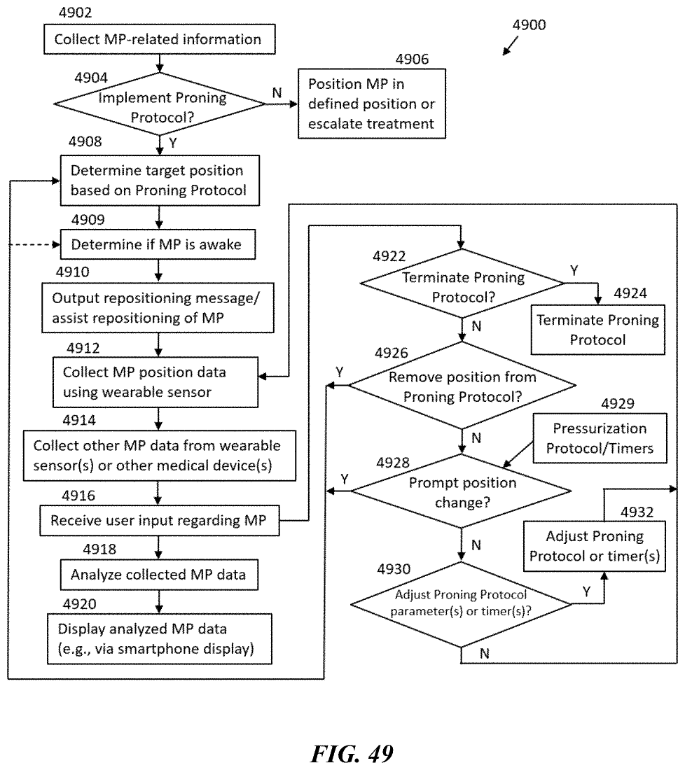

FIGS. 48A-48D illustrate example screen views generated by proning user application 4220 and displayed via GUI 4676 at user device 4210, according to one example embodiment. FIG. 48A shows a screen view 4810 showing an education menu allowing a PUA user to select different topics for learning about proning and proning regimens. FIG. 48B shows a screen view 4820 showing an menu allowing a PUA user to select from multiple predefined or stored proning protocols. In some embodiments, the proning user application 4220 allows the PUA to select a predefined proning protocol to implement for the MP. In other embodiments, the proning user application 4220 allows the PUA to select a predefined proning protocol, and then modify or customize the selected predefined protocol for the particular MP based on MP-related data input by the PUA and/or sensor data related to the MP, as discussed above. In some embodiments, the proning user application 4220 allows the PUA to create an MP-specific proning protocol independent of predefined proning protocols.

FIG. 48C shows a screen view 4830 showing the MP's performance during execution of the proning protocol selected or created for the MP. The example screen view 4830 shows (a) the selected proning protocol being implemented (“Hopkins Protocol’), (b) an accumulated time counter for a prone position and a target remaining time in the prone position, (c) the time when the MP entered the prone position, and (d) the projected time for the next position change (e.g., from the prone position to a non-prone position) specified by the proning protocol.

FIG. 48D shows a screen view 4840 showing historical data regarding the MP's performance with respect to the proning protocol.

FIG. 49 illustrates an example method 4900 for implementing and managing a proning protocol for a monitored person (MP), according to one example embodiment. As 4902, information is collected regarding the MP in any suitable manner, e.g., (a) by accessing stored medical records, lab tests, etc. regarding the MP, (b) obtaining information from an MP questionnaire or conversation(s) with the MP or associated person(s), e.g., family member(s), (c) from a physical assessment of the MP performed by a medical profession, e.g., including subjective observations and/or testing using medical testing devices or equipment, (d) by collecting sensor data from wearable sensor(s) worn by the MP or other sensor(s) configured to collect data regarding the MP, and/or (e) from any other source of information regarding the MP. Some or all information regarding the MP collected at 4902 may be input into a computer configured to execute suitable software to automatically analyze such data to determine whether to implement a proning protocol for the MP. Alternatively, information collected at 4902 may be analyzed mentally (e.g., by a doctor or other medical professional) or otherwise analyzed to determine whether to implement a proning protocol for the MP.

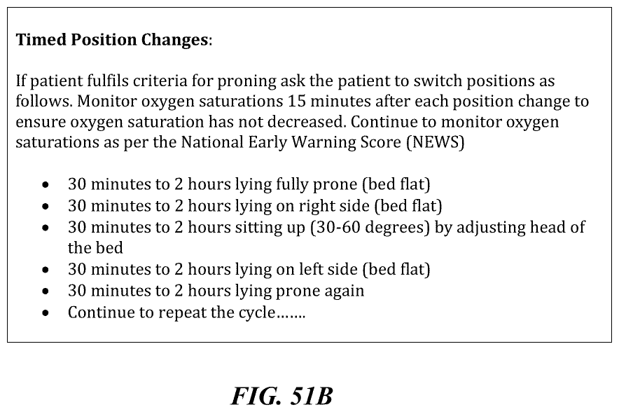

At 4904, analysis of the information collected at 4902 and/or other relevant information is performed, e.g., by a computer executing suitable software or by a medical professional performing a manual/mental analysis, to determine whether to implement a proning protocol for the MP. In some embodiments, the data collection at 4902 and analysis at 4904 may involve aspects of a known proning process flow, e.g., steps 5102-5106 of the example process flow 5100 for COVID-19 scenarios shown in FIG. 51A, discussed below. In one embodiment, a proning management system (“PM system”) including a proning user application 4220, e.g., any of the example systems shown in FIGS. 42-48D, may be configured to (a) facilitate execution of step 4902 by facilitating collection and/or entry of MP-related data relevant to steps 5102-5106 of process flow 5100, and (b) perform step 4904 by performing an automated analysis of the relevant data at each of steps 5102-5106, to thereby determine whether to implement a proning protocol for the MP (e.g., the proning protocol implemented at steps 5108-5114 of process flow 5100).

If it is determined at 4904 not to implement a proning protocol for the MP, the method proceeds to 4906, at which the MP may be positioned in a selected orientation (e.g., supine) and/or treatment for the MP may be escalated. Alternatively, if it is determined at 4904 to implement a proning protocol for the MP, the method proceeds to 4908-4932, wherein the PM system, e.g., executing a proning user application 4220, may implement and manage a proning protocol for the MP. In some embodiments, at 4904 the PM system may select a particular proning protocol for the MP from multiple available proning protocols, and/or may select or set one or more parameters of a proning protocol for the MP, based on the collected data regarding the MP. Such proning protocol parameters may include, for example, any one or more of the example proning protocol parameters listed above in the description of FIG. 44.

The PM system (including proning user application 4220) may then implement and manage the selected proning protocol at steps 4908-4932. For example, in some embodiments a proning user application 4220 (e.g., provided at a user device 4210, e.g., smartphone) may receive sensor data from at least one wearable sensor 4320 on the MP and/or other sensor(s) monitoring the MP and analyze such data to implement and manage the selected proning protocol.

At 4908, the proning user application 4220 may determine a first target body position for the MP based on the selected proning protocol, for example a prone position. At 4909, the proning user application 4220 may determine whether the MP is awake or asleep, e.g., based on sensor data received from one or more sensors 4640 of at least one wearable sensor 4320 and/or other sensor data from sensor(s) and/or other medical device(s) monitoring the MP. For example, application 4220 may analyze acceleration data from at least one accelerometer 4642 and/or audio data from a microphone or other audio sensor 4648 in at least one wearable sensor 4320 (e.g., to analyze heart rate and/or respiratory activity of the MP), heart rate data from EKG electrodes connected to the MP), respiration data from a respirator, ventilator, or other respiration sensor or monitor connected to the MP, and/or any other types of sensor(s) or medical devices configured to generated data regarding the MP that may be used to analyze whether the MP is awake or asleep. In some embodiments, step 4909 may be omitted.

At 4910, the proning user application 4220 system may cause a human-perceptible repositioning message to be output to the PM, caretaker(s), and/or other person(s), e.g., via an output device of wearable sensor 4320 or user device (e.g., smartphone) 4210, and/or via at least one CT device 4310 in an implementation involving CT device(s) (e.g., based on communication between proning user application 4220 and proning management application 4330 at each CT device 4310). The repositioning message output at any respective device (e.g., wearable sensor 4320, user device 4210, or CT device 4310) may be a visual, audible, or haptic message, e.g., based on (a) the types of output device(s) provided at the respective device, (b) the type or importance of the particular repositioning message, (c) whether the MP is awake or asleep, or (d) any other relevant factor. The repositioning message may indicate a need or recommendation for repositioning of the MP, e.g., at the present time or at a specified time (e.g., in 2 minutes from the present), and may indicate the target position determined at 4908.

In embodiments in which proning user application 4220 is configured to determine whether the MP is awake or asleep at 4909, application 4220 may control which device(s) will output the repositioning message and/or the type of the repositioning message, depending on whether the MP is determined to be awake or asleep. For example, if the application 4220 determines the MP is awake, a repositioning message may be output at the wearable sensor 4320 or user device 4210 nearby the MP, such that the MP may reposition themselves to the relevant target body position. Alternatively, if the application 4220 determines the MP is asleep, application 4220 may (a) in an implementation involving CT devices (e.g., in a hospital setting), notify a CT device 4310 to output a repositioning message to prompt a caretaker to reposition the MP to the relevant target body position or (b) in an implementation not involving CT devices (e.g., in a home setting), output a wake alarm intended to wake the MP, followed by the repositioning message prompting the MP to reposition themselves to the relevant target body position.

After the reposition message is output at 4910, the MP may (or may not) then be repositioned to the relevant target position, either on their own accord or assisted by a caregiver or other person.

At 4912-4918, the proning user application 4220 may collect various data regarding the MP relevant to managing the implementation of the proning protocol. For example, at 4912 at least one accelerometer, magnetometer, and/or other type of sensor(s) provided in at least one wearable sensor 4320 secured to the MP may generate sensor data regarding the MP's physical orientation, and communicate such orientation-related sensor data to the proning user application 4220, e.g., via wireless link(s) between the wearable sensor(s) 4320 and the user device 4210 hosting the proning user application 4220.

At 4914, at least one other sensor or medical device configured to monitor physiological data regarding the MP, e.g., sensor(s) provided in a wearable device 4320 and/or other sensor(s) or other device(s) configured to monitor the MP, may generate data regarding at least one physiological parameter of the MP other than the MP's physical orientation, and communicate such data to the proning user application 4220, e.g., via wireless link(s) with the user device 4210 hosting the proning user application 4220. For example, any of the types of sensors 4640 provided in the wearable sensor 4320 shown in FIG. 46 may generate and communicate sensor data to proning user application 4220. As another example, other sensors or medical devices, e.g., a fingertip pulse oximeter 4520 (see, e.g., FIG. 45) or other oximeter, an EKG or other heart monitoring device, and/or a respirator or ventilator connected to the MP, may monitor various physiological parameters of the MP (e.g., SpO2, SaO2, FiO2, PaCO2, respiratory rate, heart rate, heart rate variation (HRV), SBP, etc.) and communicate such data to proning user application 4220.

At 4916, user input regarding the MP may be received, e.g., via at least one user interface provided at wearable sensor 4320, user device 4210, or other computer or medical device, and communicated to the proning user application 4220. User input may be received from the MP, a caretaker, or other person. For example, a user may enter the MP's current body position via GUI 4676 of user device 4210, e.g., for calibration of orientation analysis by proning user application 4220. As another example, a user may enter a physiological parameter of the MP that is not automatically monitored in the particular implementation, e.g., oxygen saturation data from a fingertip oximeter (where the oximeter is not configured to communicate with wearable sensor 4320 or user device 4210), or data regarding heat rate, respiratory rate, coughing, wheezing, or other data that not automatically monitored by a sensor or other medical device. As another example, the MP may input a discomfort or pain level via GUI 4676 of user device 4210. In some embodiments, one or more of steps 4912, 4914, and 4916 may be omitted from the method.

At 4918, proning user application 4220 may analyze data collected at 4912-4916 for various purposes, e.g., to generate information to display via the user device display 4674, to analyze the MP's body position, or to determine whether to trigger a defined action, e.g., any of the actions specified in steps 4922-4932. At 4920, proning user application 4220 may display various data, e.g., via LEDs 4620 or other display element(s) of wearable sensor 4320, via a GUI 4676 displayed at display device 4674 of user device 4210, and/or via a GUI or other display provided at a CT device 4310. For example, proning user application 4220 may display the types of information shown in example FIGS. 48C and 48D, or any other information relevant to execution of the proning protocol or otherwise related to the MP.

In addition to generating and outputting visual feedback at 4920, the analysis of the collected data at 4918 may also be used to make various determinations and/or adjustments related to the proning protocol being executed.

At 4922, proning user application 4220 may determine whether to terminate the current execution of the proning protocol. For example, proning user application 4220 may compare selected data collected at any step 4912-4916 with one or more predefined protocol termination threshold values. For instance, application 4220 may compare an O₂Sat value (e.g., SpO₂ or SaO₂ value) obtained at any step 4912-4916 with a defined minimum O₂Sat protocol termination threshold value, and if the O₂Sat value is below the minimum O₂Sat protocol termination threshold value, determine to terminate the proning protocol at 4924, and output a proning protocol termination message, e.g., via a visual, audible, or haptic output device of wearable sensor 4320 or user device (e.g., smartphone) 4210. In one embodiment, different minimum O₂Sat protocol termination threshold values may be defined for different body positions.

As another example, a rate of decrease of an O₂Sat value (e.g., SpO2 or SaO₂) during a defined time period may be monitored from O₂Sat data obtained at any step 4912-4916, and such O₂Sat rate-of-decrease may be compared with a defined O₂Sat rate-of-decrease protocol termination threshold value. If the O₂Sat rate-of-decrease exceeds the O₂Sat rate-of-decrease protocol termination threshold value, application 4220 may determine to terminate the proning protocol at 4924, and output a proning protocol termination message, e.g., as discussed above.

In other embodiments, at 4922 proning user application 4220 may compare (a) any one or more other physiological parameters of the MP obtained at any step 4912-4916 (e.g., SpO₂, SaO₂, FiO₂, PaCO₂, respiratory rate, heart rate, heart rate variation (HRV), SBP, etc.), or (b) any parameters calculated or determined from such data obtained at any step 4912-4916 (e.g., a rate of increase, a rate of decrease, or other defined historical trend of any of SpO₂, SaO₂, FiO₂, PaCO₂, respiratory rate, heart rate, heart rate variation (HRV), SBP, etc. over a defined period of time), with predefined protocol termination threshold value(s) to determine whether to terminate the proning protocol at 4924.

At 4926, proning user application 4220 may determine, based on data collected at any step 4912-4916, whether to remove or exclude a particular body position (e.g., the current position of the MP) from the set of body positions included in the current proning protocol. For example, proning user application 4220 may compare selected data collected at any step 4912-4916 with one or more predefined position exclusion threshold values. For instance, application 4220 may compare an O₂Sat value (e.g., SpO₂ or SaO₂ value) obtained at any step 4912-4916 with a defined position exclusion threshold value, and if the oxygen saturation value is below the defined position exclusion threshold value, application 4220 may determine to remove or exclude the current body position from the set of body positions included in the current proning protocol, and may output a notification message, e.g., via a visual or audible output device of wearable sensor 4320 or user device (e.g., smartphone) 4210 indicating the removal or exclusion of the body position from the proning protocol. As another example, application 4220 may compare a rate of decrease of an O₂Sat value (e.g., SpO₂ or SaO₂ value) based on data obtained at any step 4912-4916 with a defined position exclusion threshold value, and if the O₂Sat value rate-of-decrease is below the position exclusion threshold value, application 4220 may determine to remove or exclude the current body position from the from the proning protocol, and output a corresponding notification message.

As another example, at 4926 application 4220 may compare O₂Sat values determined for multiple different body positions, and determine whether to remove or exclude a particular body position (or multiple body positions) from the current proning protocol based on the comparison of the respective O₂Sat values determined for the different body positions. For instance, application 4220 may remove or exclude a particular body position if the determined O₂Sat value for the particular body position differs from the determined O₂Sat values for other body positions by a defined threshold amount. The O₂Sat value determined for each different body position (which may be compared against each other, as discussed above) may include, for example, (a) an average O₂Sat value for each different body position, or (b) an O₂Sat value detected at a defined time in each different body position (e.g., 10 minutes after moving to the respective body position), or (c) an O₂Sat rate-of-decrease or increase during a defined time in each different body position (e.g., during the first 10 minutes spent in each respective body position).

In some embodiments, the position exclusion threshold value(s) applied at 4926 may have different values than corresponding protocol termination threshold value(s) applied at 4922, such that the magnitude of a particular physiological parameter value (e.g., O₂Sat value or O₂Sat rate-of-decrease) may be insufficient to trigger the termination of the proning protocol execution at 4922, but sufficient to trigger the removal or exclusion of a particular body position from the proning protocol at 4926.

As shown in FIG. 49, if application 4220 determines to remove or exclude the current body position from the current proning protocol at 4926, the method may return to 4908 for application 4220 to determine a new target position for the now-modified proning protocol. Otherwise, the method may proceed to 4928.

At 4928, proning user application 4220 may determine, based on data collected at any step 4912-4916, whether to prompt a position change of the MP, according to the rules and parameters of the proning protocol. For example, proning user application 4220 may first determine whether the MP is positioned in the current target body position determined at 4908. If not, application 4220 may return to 4909 and 4910 to determine whether the MP is awake or asleep (optional) and output an appropriate notification message, e.g., via a visual, audible, or haptic output device of wearable sensor 4320 or user device (e.g., smartphone) 4210 prompting the MP to be repositioned to the target body position. Otherwise, if application 4220 determines the MP is positioned in the current target body position, application 4220 may determine whether to prompt a body position change (repositioning) based on various factors, including the amount of time spent by the MP in the current body position and/or other body positions. In some embodiments, proning user application 4220 may maintain a body position timer or timers indicating time spent by the MP in each different body position involved in the proning protocol, based on sensor-based MP position data collected at 4912 (e.g., based on data from at least one accelerometer, magnetometer, and/or other orientation-related or position-related sensor(s) in wearable sensor(s) 4320 secured to the MP), and determine whether to prompt a position change based on the value(s) of one or more body position timer.

For example, proning user application 4220 may maintain a continuous body position timer indicting the continuous duration of time spent in the current body position. Proning user application 4220 may also maintain a cumulative body position timer for each different body position indicating a cumulative amount of time spent in each respective body position, for example during a defined period of time (e.g., the previous 8 hours from the present time), or spanning a defined number of discrete sessions spent in the respective body position (e.g., the most recent 3 sessions spent in the respective body position). In some embodiments, a continuous body position timer and/or cumulative body position timer for each respective body position may count up at a defined rate of increase while the MP is in the respective body position, and count down at a defined rate of decrease while the MP is not in the respective body position. In other embodiments, a continuous body position timer and/or cumulative body position timer for each respective body position may count down (e.g., towards zero) at a defined rate of decrease while the MP is in the respective body position, and count up at a defined rate of increase while the MP is not in the respective body position. In such embodiments, a continuous body position timer and/or cumulative body position timer for each respective body position may have defined rate of increase that differs from (e.g., higher or lower) the defined rate of decrease. Further, the defined rate of increase and/or the defined rate of decrease may be different for the body position timers for different body positions. In addition, as discussed below regarding step 4930, in some embodiments application 4220 may adjust the defined rate of increase and/or the rate of decrease for one or more body position timers (corresponding to one or more different body positions) based on particular data collected at any step 4912-4916.

Proning user application 4220 may determine at 4928 whether to prompt a position change based on the value(s) of one or more body position timer (e.g., any of the example types of body position timers discussed above) according to any suitable rules or algorithms. For example, application 4220 may determine to prompt a position change if the value of a body position timer for the current body position of the MP (e.g., a continuous body position timer or a cumulative body position timer for the current body position) meets a defined threshold time value for the current body position, as specified by the proning protocol. As used herein, “meeting” a particular threshold value may include matching the particular threshold value or crossing the particular threshold value, either in an increasing or decreasing direction, as defined by the proning protocol for the particular threshold value. For example, application 4220 may determine to prompt a position change if an upwardly-counting body position timer for the current body position reaches a defined threshold time value (e.g., 2 hours), or if a downwardly-counting current body position timer counts down to a defined threshold time value (e.g., zero).

In some embodiments, proning user application 4220 may also incorporate aspects of a pressurization-based turning protocol, for example any of the pressurization-based timers (e.g., cumulative timers for respective positions) or turning protocols discussed above, indicated at 4929, for determining whether to prompt a position change at 4928. For example, proning user application 4220 may determine whether to prompt a position change based on both (a) proning protocol rules designed to encourage time spent in the prone position and (b) pressurization-based rules designed to avoid or reduce the likelihood of pressure ulcer development. Thus, proning user application 4220 may prompt a proning-related position change only if such position change conforms with pressurization-based rules. For instance, proning user application 4220 may prompt a proning-related position change to a particular position (e.g., prone position) only if an accumulated pressurization timer for the particular position (e.g., prone position) is below a defined pressurization threshold or otherwise within defined pressurization-related limits.

Application 4220 may then, at 4908, determine a target body position for the prompted repositioning of the MP based on any suitable rules or parameters of the proning protocol, determine whether the MP is awake or asleep at 4909 (optional), and output a notification of the target body position at 4910. For example, application 4220 may determine a target body position based on a positioning schedule or sequential order of body positions defined by the proning protocol. For instance, the proning protocol may determine the target body position as the next position in the following defined sequence of body positions: (1) lying prone (with bed flat), (2) lying on right side (with bed flat), (3) sitting up (with head of bed at 30-60 degree incline), (4) lying on left side (with bed flat), (5) return to step (1) and repeat the cycle. As another example, application 4220 may determine the target body position based on the respective timer values for each of the different body positions included in the proning protocol. For example, application 4220 may determine the target body position as the body position having the lowest cumulative timer value, i.e., representing the body position in which the MP has spent the least amount of time over some defined time period (e.g., the last 6 hours). As another example, application 4220 may determine the target body position as the body position that the MP has not been in (or has not spent a defined minimum duration, e.g., 10 minutes) for the greatest amount of time from the present, in other words the body position that the MP has been out of for the longest time from the present time.

If application 4220 determines at 4928 that a position change is not needed, the method may proceed to 4930. At 4930, application 4220 may determine whether to adjust one or more proning protocol parameters and/or body position timers at 4932 based on data collected at any step 4912-4916. For example, as noted above regarding step 4928, application 4220 may adjust the defined rate of increase and/or the rate of decrease for one or more body position timers (corresponding to one or more different body positions) based on data collected at any step 4912-4916. For example, application 4220 may adjust (increase or decrease) the rate of increase for a continuous body position timer and/or cumulative body position timer for the current position of the MP as a function of sensor-detected O₂Sat data, respiratory rate data, heart rate data, body temperature data, etc. As another example, application 4220 may adjust (increase or decrease) the minimum or maximum duration to be spent in one or more particular body positions (e.g., the minimum or maximum duration to be spent in the prone position) based on the current O₂Sat data or O₂Sat data trend, current respiratory rate or respiratory rate trend, heart rate, HRV, or other heart rate trend, or any other data collected at 4912-4916.

It should be understood that steps 4922-4932 may be performed in any order and/or at any frequency relative to time or relative to the performance of any of steps 4912-4920. Further, in some embodiments any of steps 4922-4932 may be omitted from method 4900.

FIG. 50 illustrates an example method 5000 for implementing and managing a proning protocol for a monitored person (MP), according to one example embodiment. As 5002, information is collected regarding the MP in any suitable manner, e.g., as discussed above regarding step 4902 of method 4900.

At 5004, analysis of the information collected at 5002 and/or other relevant information is performed, e.g., by a computer executing suitable software or by a medical professional performing a manual/mental analysis, to determine whether to implement a proning protocol for the MP, e.g., as discussed above regarding step 4904 of method 4900.

If it is determined at 5004 not to implement a proning protocol for the MP, the method proceeds to 5006, at which the MP may be positioned in a selected orientation (e.g., supine) and/or treatment for the MP may be escalated. Alternatively, if it is determined at 5004 to implement a proning protocol for the MP, the method proceeds to 5008-5032, wherein the PM system, e.g., executing a proning user application 4220, may implement and manage a proning protocol for the MP. In some embodiments, at 5004 the PM system may select a particular proning protocol for the MP from multiple available proning protocols, and/or may select or set one or more proning protocol parameters, e.g., any of the example proning protocol parameters disclosed above, based on the collected data regarding the MP.

The PM system (including proning user application 4220) may then implement and manage the selected proning protocol at steps 5008-5036. For example, in some embodiments a proning user application 4220 (e.g., provided at a smartphone or other user device 4210) may receive sensor data from at least one wearable sensor 4320 on the MP and/or other sensor(s) monitoring the MP and analyze such data to implement and manage the selected proning protocol.

At 5008, the proning user application 4220 may determine a first target body position for the MP based on the selected proning protocol, for example a prone position. At 5009, the proning user application 4220 may determine whether the MP is awake or asleep, e.g., as discussed above regarding step 4902 of method 4900. In some embodiments, step 5009 may be omitted.

At 5010, the proning user application 4220 system may cause a human-perceptible repositioning message to be output to the PM, caretaker(s), and/or other person(s), e.g., via wearable sensor 4320, user device 4210, or at least one CT device 4310, e.g., as discussed above regarding step 4910 of method 4900. After the reposition message is output at 5010, the MP may (or may not) then be repositioned to the relevant target position, either on their own accord or assisted by a caregiver or other person.

At 5012, at least one accelerometer, magnetometer, and/or other type of sensor(s) provided in at least one wearable sensor 4320 secured to the MP may generate sensor data regarding the MP's physical orientation, and communicate such orientation-related sensor data to the proning user application 4220, e.g., via wireless link(s) between the wearable sensor(s) 4320 and the user device 4210 hosting the proning user application 4220.

At 5014, proning user application 4220 may maintain and manage a body position timer or timers indicating time spent by the MP in one, some, or each different body position involved in the proning protocol, e.g., as discussed above regarding step 4918 of method 4900. For example, application 4220 may maintain and manage body position timer(s) based on sensor-based MP position data collected at 5012 (e.g., based on data from at least one accelerometer, magnetometer, and/or other orientation-related or position-related sensor(s) in wearable sensor(s) 4320 secured to the MP), and may determine at 5016 (discussed below) whether to prompt a position change based on the value(s) of one or more body position timer. For example, at 5014, application 4220 may maintain a continuous body position timer indicting the continuous duration of time spent in the current body position. Application 4220 may also maintain a cumulative body position timer for each different body position indicating a cumulative amount of time spent in each respective body position, e.g., as discussed above regarding step 4918 of method 4900. As discussed above, in some embodiments each body position timer may count up at a defined rate of increase while the MP is in the respective body position, and count down at a defined rate of decrease while the MP is not in the respective body position. In other embodiments, each body position timer may count down (e.g., towards zero) at a defined rate of decrease while the MP is in the respective body position, and count up at a defined rate of increase while the MP is not in the respective body position. In such embodiments, a continuous body position timer and/or cumulative body position timer for each respective body position may have defined rate of increase that differs from (e.g., higher or lower) the defined rate of decrease. Further, the defined rate of increase and/or the defined rate of decrease may be different for the body position timers for different body positions. In addition, as discussed below regarding step 5034, in some embodiments application 4220 may adjust the defined rate of increase and/or the rate of decrease for one or more body position timers (corresponding to one or more different body positions) based on particular data collected at any step 5012, 5018, 5020, 5022.

As noted above, at 5016, proning user application 4220 may determine, based on orientation data collected at step 5012 and one or more body positions timers managed at 5014, whether to prompt a position change of the MP, according to the rules and parameters of the proning protocol. For example, application 4220 may first determine whether the MP is positioned in the current target body position determined at 5008. If not, application 4220 may return to 5009 and 5010 to determine whether the MP is awake or asleep (optional) and output an appropriate notification message, e.g., via a visual, audible, or haptic output device of wearable sensor 4320 or user device (e.g., smartphone) 4210 prompting the MP to be repositioned to the target body position. Otherwise, if application 4220 determines the MP is positioned in the current target body position, application 4220 may determine whether to prompt a body position change (repositioning) based on the monitored body position of the MP, including the amount of time spent by the MP in the current body position and/or other body positions, e.g., as indicated by the body position timer(s) managed at 5014.

Application 4220 may determine at 5016 whether to prompt a position change based on the value(s) of one or more body position timer managed at 5014 according to any suitable rules or algorithms. For example, application 4220 may determine to prompt a position change if the value of a body position timer for the current body position of the MP (e.g., a continuous body position timer or a cumulative body position timer for the current body position) meets a defined threshold time value for the current body position, as specified by the proning protocol. For example, application 4220 may determine to prompt a position change if an upwardly-counting body position timer for the current body position reaches a defined threshold time value (e.g., 2 hours), or if a downwardly-counting current body position timer counts down to a defined threshold time value (e.g., zero).

If application 4220 determines to prompt a position change, application 4220 may then return to 5008 to determine a target body position for the prompted repositioning of the MP based on any suitable rules or parameters of the proning protocol, determine whether the MP is awake or asleep at 5009 (optional), and output a notification of the target body position at 5010. For example, application 4220 may determine a target body position based on a positioning schedule or sequential order of body positions defined by the proning protocol. For instance, the proning protocol may determine the target body position as the next position in the following defined sequence of body positions: (1) lying prone (with bed flat), (2) lying on right side (with bed flat), (3) sitting up (with head of bed at 30-60 degree incline), (4) lying on left side (with bed flat), (5) return to step (1) and repeat the cycle. As another example, application 4220 may determine the target body position based on the respective timer values for each of the different body positions included in the proning protocol. For example, application 4220 may determine the target body position as the body position having the lowest cumulative timer value, i.e., representing the body position in which the MP has spent the least amount of time over some defined time period (e.g., the last 6 hours). As another example, application 4220 may determine the target body position as the body position that the MP has not been in (or has not spent a defined minimum duration, e.g., 10 minutes) for the greatest amount of time from the present, in other words the body position that the MP has been out of for the longest time from the present time.

If application 4220 determines at 5016 that a position change is not needed, the method may proceed to 5018. At 5018, the MP's oxygen saturation (e.g., SpO2 or SaO2) may be measured, e.g., by a O₂Sat sensor provided in wearable sensor 4320, or by a fingertip pulse oximeter 4520 (see, e.g., FIG. 45), or other oximeter sensor or device, and the measured O₂Sat data may be communicated to the proning user application 4220, e.g., via wireless link(s) with the user device 4210 hosting the proning user application 4220. Alternatively, the measured O₂Sat data may be manually input, e.g., by a caretaker, into a suitable GUI provided by proning user application 4220, or at another digital interface communicatively connected to proning user application 4220.

At 5020, the respiratory data of the MP may be monitored, e.g., respiratory rate (RR), tidal volume, respiratory oscillations, or other respiratory pattern parameters. Such respiratory data may be measured and monitored based on any suitable sensor data or monitored data, e.g., based on acceleration data generated by one or more accelerometers 4642 provided in wearable sensor(s) 4320, or based on thoracic acoustic data or tracheal acoustic data generated by microphone(s) or other audio sensor(s) 4648 provided in wearable sensor(s) 4320, wherein such sensor data may be wirelessly communicated from the wearable sensor(s) 4320 to the user device 4210 hosting the proning user application 4220. Alternatively, the relevant respiratory data (e.g., respiratory rate, etc.) may be monitored by another sensor or medical device, e.g., a respirator, ventilator, thoracic circumference sensor configured to measuring fluctuations in thoracic circumference with respiration, VT electrodes configured to measure fluctuations in lung volume with respiration, EKG sensor, PPG sensor (e.g., measured by a pulse oximeter), or any other suitable sensor or device. Such sensor data may be automatically communicated (e.g., via wireless or wired connection) to the user device 4210 hosting the proning user application 4220, or may be manually input, e.g., by a caretaker, into a suitable GUI provided by proning user application 4220, or at another digital interface communicatively connected to proning user application 4220.

At 5022, proning user application 4220 may analyze data collected at 5018 and 5020 for various purposes, e.g., to generate information to display at 5024 and/or to determine whether to trigger one or more defined actions at 5024-5036. At 5024, proning user application 4220 may display various data, e.g., via LEDs 4620 or other display element(s) of wearable sensor 4320, via a GUI 4676 displayed at display device 4674 of user device 4210, and/or via a GUI or other display provided at a CT device 4310. For example, proning user application 4220 may display the types of information shown in example FIGS. 48C and 48D, or any other information relevant to execution of the proning protocol or otherwise related to the MP.

In addition to generating and outputting visual feedback at 5024, the data analysis at 5022 may also be used to make various determinations and/or adjustments related to the proning protocol being executed. At 5026, application 4220 may determine whether to terminate the current execution of the proning protocol based on the O₂Sat data measured at 5018 and/or respiratory data (e.g., RR, etc.) measured at 5020. For example, proning user application 4220 may compare O₂Sat data and/or respiratory data (e.g., RR) with one or more predefined protocol termination threshold values. For instance, application 4220 may compare an O₂Sat value (e.g., SpO2 or SaO2 value) monitored at 5018 with a defined minimum O₂Sat protocol termination threshold value, and if the O₂Sat value is below the minimum O₂Sat protocol termination threshold value, determine to terminate the proning protocol at 5028, and output a proning protocol termination message, e.g., via a visual, audible, or haptic output device of wearable sensor 4320 or user device (e.g., smartphone) 4210. As another example, application 4220 may compare the MP's respiratory rate (RR) monitored at 5020 with a defined minimum RR protocol termination threshold value and a defined maximum RR protocol termination threshold value, and if the monitored RR value is below the minimum RR protocol termination threshold value or above the maximum RR protocol termination threshold value, determine to terminate the proning protocol at 5028, and output a proning protocol termination message. In one embodiment, different protocol termination threshold values (for O₂Sat and/or RR) may be defined for different body positions.

As another example, a rate of decrease of an O₂Sat value (e.g., SpO2 or SaO2) during a defined time period may be monitored from O₂Sat data monitored at 5018, and such O₂Sat rate-of-decrease may be compared with a defined O₂Sat rate-of-decrease threshold value. If the O₂Sat rate-of-decrease exceeds the defined O₂Sat rate-of-decrease threshold value, application 4220 may determine to terminate the proning protocol at 5026, and output a proning protocol termination message, e.g., as discussed above. As another example, a rate of RR increase or RR decrease during a defined time period may be monitored from RR data monitored at 5020, and such RR rate-of-increase or RR rate-of-decrease may be compared with a respective RR rate-of-increase threshold value or RR rate-of-decrease threshold value, to determine whether to terminate the proning protocol at 5026.

At 5030, proning user application 4220 may determine, based on the O₂Sat data measured at 5018 and/or respiratory data (e.g., RR, etc.) measured at 5020, along with orientation data collected at 5012 (e.g., indicating the current body position of the MP), whether to remove or exclude a particular body position (e.g., the current position of the MP) from the set of body positions included in the current proning protocol. For example, proning user application 4220 may compare O₂Sat data and/or respiratory data collected at 5018-5020 with one or more predefined position exclusion threshold values. For instance, application 4220 may compare an O₂Sat value (e.g., SpO2 or SaO2 value) obtained at 5018 with a defined position exclusion threshold value, and if the O₂Sat value is below the defined position exclusion threshold value, application 4220 may determine to remove or exclude the current body position from the set of body positions included in the current proning protocol, and may output a notification message, e.g., via a visual or audible output device of wearable sensor 4320 or user device (e.g., smartphone) 4210 indicating the removal or exclusion of the body position from the proning protocol. As another example, application 4220 may compare the MP's respiratory rate (RR) monitored at 5020 with a defined minimum RR position exclusion threshold value and/or a defined maximum RR position exclusion threshold value, and if the monitored RR value is below the minimum RR position exclusion threshold value or above the maximum RR position exclusion threshold value, determine to remove or exclude the current body position from the set of body positions included in the current proning protocol, and output a notification message. As another example, application 4220 may compare any of an O₂Sat value rate-of-decrease, an RR value rate-of-decrease, or an RR value rate-of-increase based on data obtained at steps 5018-5020 with a respective position exclusion threshold value, and based on the results of such comparison, determine to remove or exclude the current body position from the from the proning protocol, and output a corresponding notification message.

As another example, at 5030 application 4220 may compare O₂Sat values or RR values determined for multiple different body positions, and determine whether to remove or exclude a particular body position (or multiple body positions) from the current proning protocol based on the comparison of the respective O₂Sat values or RR values determined for the different body positions. For instance, application 4220 may remove or exclude a particular body position if the determined O₂Sat value or RR value for the particular body position differs from the determined O₂Sat values or RR value for other body positions by a defined threshold amount. The O₂Sat value or RR value determined for each different body position may include, for example, (a) an average O₂Sat value or RR value for each different body position, or (b) an O₂Sat value of RR value detected at a defined time in each different body position (e.g., 10 minutes after moving to the respective body position), or (c) an O₂Sat rate-of-decrease or rate-of-increase, or RR rate-of-decrease or rate-of-increase, during a defined time in each different body position (e.g., during the first 10 minutes spent in each respective body position).

In some embodiments, the position exclusion threshold value(s) applied at 5030 may have different values than corresponding protocol termination threshold value(s) applied at 5026, such that the magnitude of a particular physiological parameter value (e.g., O₂Sat value, O₂Sat rate-of-decrease, RR value, RR rate-of-decrease, or RR rate-of-increase) may be insufficient to trigger the termination of the proning protocol execution at 5026, but sufficient to trigger the removal or exclusion of a particular body position from the proning protocol at 5030.

As shown in FIG. 50, if application 4220 determines to remove or exclude the current body position from the current proning protocol at 5030, the method may return to 5008 for application 4220 to determine a new target position for the now-modified proning protocol. Otherwise, the method may proceed to 5032.

At 5032, proning user application 4220 may determine, based on the O₂Sat data measured at 5018 and/or respiratory data (e.g., RR, etc.) measured at 5020, along with orientation data collected at 5012 (e.g., indicating the current body position or historical position data of the MP), whether to prompt a position change of the MP, according to the rules and parameters of the proning protocol.

For example, proning user application 4220 may compare O₂Sat data and/or respiratory data collected at 5018-5020, e.g., O₂Sat values, O₂Sat rate-of-increase, O₂Sat rate-of-decrease, RR values, RR rate-of-increase, and/or RR rate-of-decrease, with one or more predefined repositioning threshold values, e.g., similar to any of the example comparisons of monitored data to threshold values discussed above at 5026 or 5030. Based on the results of such comparison(s) at 5032, application 4220 may determine to prompt a repositioning of the MP, and thus the method may return to 5008 to determine a new target body position. In some embodiments, the repositioning threshold values applied at 5030 may have different values than corresponding protocol termination threshold values applied at 5026 and position exclusion threshold values applied at 5030, such that the magnitude of a particular physiological parameter value (e.g., O₂Sat value, O₂Sat rate-of-increase, O₂Sat rate-of-decrease, RR value, RR rate-of-decrease, or RR rate-of-increase) may be insufficient to trigger the termination of the proning protocol execution at 5026 or removal of the current body position from the proning protocol at 5030, but sufficient to trigger a position change at 5032.

In some embodiments, proning user application 4220 may also incorporate a pressurization-based turning protocol, for example including any of the pressurization-based timers (e.g., cumulative timers for respective positions) and turning protocols disclosed herein, indicated at 5033, for determining whether to prompt a position change at 5032. For example, proning user application 4220 may determine whether to prompt a position change based on both (a) proning protocol rules designed to encourage time spent in the prone position and (b) pressurization-based rules designed to avoid or reduce the likelihood of pressure ulcer development. Thus, proning user application 4220 may prompt a proning-related position change only if such position change conforms with pressurization-based rules. For instance, proning user application 4220 may prompt a proning-related position change to a particular position (e.g., prone position) only if an accumulated pressurization timer for the particular position (e.g., prone position) is below a defined pressurization threshold or otherwise within defined pressurization-related limits.

If application 4220 determines at 5032 that a position change is not needed, the method may proceed to 5034. At 5034, application 4220 may determine whether to adjust one or more proning protocol parameters and/or body position timers at 5032 based on the O₂Sat data measured at 5018 and/or respiratory data (e.g., RR, etc.) measured at 5020. For example, application 4220 may adjust the defined rate of increase and/or the rate of decrease for one or more body position timers (corresponding to one or more different body positions) based on orientation data collected at 5012, O₂Sat data measured at 5018, and/or respiratory data measured at 5020. For example, application 4220 may adjust (increase or decrease) the rate of increase for a continuous body position timer and/or cumulative body position timer for the current position of the MP as a function of sensor-detected O₂Sat data (e.g., O₂Sat value, O₂Sat rate-of-increase, and/or O₂Sat rate-of-decrease) and/or respiratory data (e.g., RR value, RR rate-of-decrease, and/or RR rate-of-increase).

It should be understood that steps 5026-5036 may be performed in any order and/or at any frequency relative to time or relative to the performance of any of steps 5012-5024, e.g., in any order and/or at any frequency relative to time or relative to the orientation-based monitoring and analysis at steps 5012-5016. Further, in some embodiments any of steps 5026-5036 may be omitted from method 5000.

FIG. 51A shows an example process flow 5100 for determining whether to implement, and implementing, a proning protocol for a conscious patent, as published by the Intensive Care Society (ISC). (Peter Bamford et al., ICS Guidance for Prone Positioning of the Conscious COVID MP 2020). Any one, some, or all steps of process flow 5100, e.g., any of steps 5102-5114, may be fully or partially automated, e.g., by any of the systems shown in FIGS. 42-47. For example, the functionality of any one or more steps of process flow 5100, e.g., any of steps 5102-5114, may be performed or facilitated by a suitable proning user application 4220 and/or proning management application 4330, e.g., based on (a) physiological data and/or other data automatically received from one or more sensors or medical devices (e.g., at least one wearable sensor 4320, a pulse oximeter 4520, a ventilator or other oxygen or respiratory therapy device 4540 (in applicable situations), and/or any other type(s) of sensors or medical devices), (b) physiological data and/or other data input by a user (e.g., via a respective GUI of proning user application 4220 and/or proning management application 4330), and/or (c) physiological data and/or other data received from computer memory (e.g., stored data regarding the MP) or other computer system.

At 5102, it is determined, either automatically or by a medical professional or other person, (a) whether the MP's FiO2 is greater than or equal to 28% or the MP requires basic respiratory support to achieve SaO2 of 92-96% (or 88-92% if there is a determined risk of hypercapnic respiratory failure) and (b) there is a suspected or confirmed COVID-19 diagnosis for the MP. In some embodiments or situations, FiO₂ and or SaO₂ values may be automatically measured or monitored by suitable sensor(s) and/or devices and automatically communicated to a proning user application 4220 or proning management application 4330 programmed to perform or facilitate the analysis at 5102. In embodiments or situations, FiO₂ and or SaO₂ values may be measured by a medical professional or other person, and input into a relevant application 4220 or 4330 programmed to perform or facilitate the analysis at 5102.

If either determination (a) or (b) at step 5012 is negative, the MP may continue in (or may be moved to) a supine position, as indicated at 5120. However, if both determinations (a) and (b) are affirmative, the method may proceed to step 5104, where a set of defined factors may be analyzed for determining whether to consider implementing a proning regimen for the MP. If the listed factors indicate a proning regimen should not be considered for the MP, the MP may continue in (or may be moved to) a supine position, as indicated at 5122. Alternatively, if the factors listed at 5104 indicate a proning regimen should be considered for the MP, the method may continue to step 5106 for further consideration of whether to implement a proning regimen.

At 5106, a set of contraindicating factors may be analyzed for determining whether to implement a proning regimen for the MP. In this example, the set of contraindicating factors includes a number of “absolute contraindications” and a number of “relative contraindications.” If any of the “absolute contraindications” indicate against implementing a proning regimen for the MP, method may proceed to 5124, where the MP may continue in (or may be moved to) a supine position and/or a determination for escalating the MP's treatment protocol may be made, e.g., by a medical professional. For the “relative contraindications,” a medical professional may consider such contraindications individually or collectively to make an experience-based judgment of whether or not implement a proning regimen for the MP. Alternatively, in some embodiments, responses or values for the relative contraindications (individually or collectively) may be analyzed with respect to defined threshold values and/or algorithms that indicate or suggest whether or not implement a proning regimen for the MP based on the relative contraindication responses or values for the MP (for example, algorithms that incorporate or integrate the responses or values for multiple relative contraindications).

In some embodiments or situations, one or more physiological parameters relevant to one or more contraindications analyzed at 5106 (e.g., RR, PaCO₂, SBP, etc.) may be automatically measured or monitored by suitable sensor(s) and/or devices and automatically communicated to a proning user application 4220 or proning management application 4330 programmed to perform or facilitate the analysis at 5102. In some embodiments or situations, physiological parameters relevant to step 5106 may be measured by a medical professional or other person, and input into a relevant application 4220 or 4330 programmed to perform or facilitate the analysis at 5102.

Upon review of the presence/absence of the contraindications listed at 5106, if it is determined to implement a proning regimen for the MP, the method may proceed to 5108-5114 for implementation and management of a proning protocol for the MP. In particular, the proning protocol may specify the timed progression of body positions defined in FIG. 51B. FIG. 51B shows an example progression of body positions defined by an example proning protocol used in the process flow of FIG. 51A, according to one example embodiment.

At 5108, the MP may be instructed, encouraged, or physically assisted to the prone position, along with the various additional actions listed at 5108. As noted, such actions including monitoring oxygen saturation (O₂Sat). In some embodiments or situations, O₂Sat data may be automatically measured or monitored by a fingertip oximeter 4520 or other oximeter or sensor configured to measure O₂Sat, and automatically communicated to a proning user application 4220 or proning management application 4330. In other embodiments or situations, O₂Sat data of the MP may be measured by a medical professional or other person, and input into application 4220 or 4330 programmed to perform or facilitate the analysis at 5108.

At 5110, after the MP enters the prone position, the MP's O₂Sat data may be monitored for a defined period. In the illustrated example the MP's SaO₂ data may be monitored for 15 minutes. As discussed above, the SaO₂ data may be automatically monitored by a fingertip oximeter 4520 or other oximeter or sensor and automatically communicated to application 4220 or application 4330 for processing, or manually measured and input into application 4220 or 4330 for processing.

As shown at step 5110, if both (a) the monitored SaO2 is maintained within the range of 92-96% (or 88-92% if the MP is determined to be at risk for hypercapnic respiratory failure) and (b) the MP exhibits no obvious distress (e.g., as determined and input to application 4220 or application 4330 by a medical professional, the MP themself, or other person monitoring the MP), the method may continue to step 5112. At 5112, the proning protocol may continue according to the timed progression of body positions defined in FIG. 51B. As noted, step 5110 (monitor SaO₂) may be performed after each position change.

Alternatively, at 5110 if either (a) the monitored SaO2 is outside, or moves outside, the range of 92-96% (or 88-92% for an MP at risk of hypercapnic respiratory failure) or (b) the MP exhibits distress, the method may continue to step 5114. At 5114, it is first determined whether the SaO2 for the MP is decreasing over time, e.g., based on the monitoring performed at 5110. If so, one or more listed actions may be taken, e.g., by a medical professional. In addition, it may be determined whether to discontinue the implementation of the proning protocol based on a number of factors, listed at 5114. One factor for determining whether to discontinue the proning protocol involves analyzing the MP's respiratory rate (RR), in particular based on whether RR≥35.

Having fully described a preferred embodiment of the invention, and numerous aspects thereof, as well as various alternatives, those skilled in the art will recognize, given the teachings herein, that numerous alternatives and equivalents exist which do not depart from the invention. It is therefore intended that the invention not be limited by the foregoing description, but only by the appended claims.

1. A method of monitoring and managing a person's orientation to encourage proning, the method comprising:
receiving orientation-related sensor data generated by at least one orientation-related sensor provided in a wearable sensor device secured to the person or to an article worn by the person;
monitoring an orientation of the person over time based on the orientation-related sensor data;
comparing the monitored orientation of the person to a defined proning protocol for the person, the proning protocol defining orientation-based rules or parameters to encourage time spent in a prone position; and
based on the comparison, outputting at least one visual or audible notification regarding time spent in the prone position.

2. The method of claim 1, wherein the at least one orientation-related sensor provided in the wearable sensor device comprises at least one accelerometer configured to generate acceleration data indicative of the orientation of the person.

3. The method of claim 1, wherein receiving orientation-related sensor data generated by at least one orientation-related sensor comprises receiving the orientation-related sensor data at a processor provided in the wearable sensor device.

4. The method of claim 1, wherein receiving orientation-related sensor data generated by at least one orientation-related sensor comprises receiving the orientation-related sensor data at a processor provided in a device separate from the wearable sensor device.

5. (canceled)

6. The method of claim 4, wherein the steps of (a) monitoring the orientation of the person over time based on the orientation-related sensor data, (b) comparing the monitored orientation of the person to the defined proning protocol, and (c) outputting the at least one visual or audible notification regarding time spent in the prone position are performed by an application provided at the device separate from the wearable sensor device.

7. The method of claim 1, comprising:
communicating the orientation-related sensor data from the wearable sensor to a mobile device; and
monitoring, by an application hosted on the mobile device, the orientation of the person over time based on the orientation-related sensor data communicated by the wearable sensor device.

8. The method of claim 1, comprising:
monitoring the orientation of the person over time, by a processor provided in the wearable sensor device, based on the orientation-related sensor data; and
communicating data indicating the monitored orientation of the person over time from the wearable sensor device to a mobile device; and
wherein the steps of (a) comparing the monitored orientation of the person to the defined proning protocol for the person and (b) based on the comparison, outputting at least one visual or audible notification regarding time spent in the prone position, are performed by an application provided by the mobile device.

9. The method of claim 1, wherein the proning protocol defines at least one of (a) a minimum duration spent in the prone position or (b) a maximum duration spent out of the prone position.

10. The method of claim 1, comprising:
receiving physiological sensor data indicating at least one of (a) respiratory data or (b) heart related data of the person; and
based at least on the physiological sensor data, automatically terminating the proning protocol for the person.

11. The method of claim 1, comprising:
receiving position-related physiological sensor data associated with a particular position of the person, the position-related physiological sensor data indicating at least one of (a) respiratory data or (b) heart related data associated with the particular position; and
based at least on the physiological sensor data, automatically removing the particular position from the proning protocol for the person.

12. The method of claim 1, comprising:
receiving physiological sensor data indicating at least one of (a) respiratory data or (b) heart related data of the person; and
based at least on the physiological sensor data, automatically outputting at least one visual or audible position change notification to encourage a position change of the person.

13. The method of claim 1, comprising:
receiving physiological sensor data generated by at least one physiological data sensor provided in the wearable sensor device, the physiological sensor data indicating at least one of (a) respiratory data or (b) heart related data of the person; and
based at least on the physiological sensor data, automatically:
terminating the proning protocol for the person;
removing a particular position from the proning protocol for the person; or
outputting at least one visual or audible position change notification to encourage a position change of the person

14. The method of claim 1, comprising:
receiving physiological sensor data by a physiological data sensor separate from the wearable sensor device, the physiological sensor data indicating at least one of (a) respiratory data or (b) heart related data of the person; and
based at least on the physiological sensor data, automatically:
terminating the proning protocol for the person;
removing a particular position from the proning protocol for the person; or
outputting at least one visual or audible position change notification to encourage a position change of the person

15. The method of claim 13, wherein the physiological sensor separate from the wearable sensor device comprises a pulse oximeter.

16. The method of claim 13, wherein the physiological sensor separate from the wearable sensor device comprises a sensor provided in a ventilator.

17. The method of claim 13, wherein the physiological sensor separate from the wearable sensor device comprises an EKG or EEG heart monitoring sensor.

18. The method of claim 13, wherein the physiological sensor separate from the wearable sensor device comprises an acoustic sensor configured to detect sounds produced by the person's body.

19. A system for monitoring and managing a person's orientation to encourage proning, the system comprising:
a wearable sensor device configured to be secured to the person or to an article worn by the person, the wearable sensor including:
at least one orientation-related sensor configured to generate orientation-related sensor data indicative of the person's orientation; and
a wireless transmitter configured to wireless transmit orientation data including the generated orientation-related sensor data or data derived from the generated orientation-related sensor data;
a mobile device separate from the wearable sensor device and including:
a wireless receiver configured to receive the orientation data transmitted by the wireless transmitter of the wearable sensor;
a mobile device processor configured to:
monitor an orientation of the person over time based on the received orientation data;
analyze the monitored orientation of the person with respect to a defined proning protocol for the person, the proning protocol defining orientation-based rules or parameters to encourage time spent in a prone position; and
generate notifications based on the analysis of the monitored orientation of the person with respect to the defined proning protocol; and
an output device configured to output human-perceptible notifications regarding time spent in the prone position.

20. (canceled)

21. The system of claim 19, wherein the proning protocol defines at least one of (a) a minimum duration spent in the prone position or (b) a maximum duration spent out of the prone position.

22. The system of claim 19, wherein:
the wireless receiver of the mobile device is further configured to receive physiological sensor data from the wearable sensor device or from another device separate from the wearable sensor device, the physiological sensor data indicating at least one of (a) respiratory data or (b) heart related data of the person; and
the mobile device processor is configured to:
analyze the physiological sensor data; and
based on the analyzed physiological sensor data:
automatically terminate the proning protocol for the person;
automatically remove the particular position from the proning protocol for the person; or
automatically output a position change notification via the output device to encourage a position change of the person.

23-26. (canceled)
